SEQUENCE LISTING (30 sequences)

SEQ ID NO: 1 - protein, 178 aa, Artificial Sequence
  Synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 1629 bp, Artificial Sequence
  Synthetic construct
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 3 - protein, 742 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 4 - protein, 699 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATSTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 5 - protein, 493 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 6 - protein, 361 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 7 - protein, 139 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCSLHCSQQSKKVWAEEKASDQQWQWSCGGQKAKWTQRRGQQVSGNGAFGEQGVVRNSRPVYDS

SEQ ID NO: 8 - protein, 429 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 9 - protein, 340 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 10 - protein, 294 aa, Artificial Sequence
  Synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRS

SEQ ID NO: 11 - DNA, 5748 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 12 - DNA, 5619 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 13 - DNA, 5001 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 14 - DNA, 4605 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 15 - DNA, 3985 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 16 - DNA, 4809 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 17 - DNA, 4542 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 18 - DNA, 2261 bp, Artificial Sequence
  Synthetic construct
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaagttgaagagattcaggttatagcataagaagagcactgtttcatcgtcttcttgctgttaggaggtctatgaagcagagaagaactttcctttggaaaacaactaaatgaagacagtcacctcgctagaactgacacatgggctgtttttatattcttgaaggccactctctccctacctgaaccaagacctataggtttacatgttatttacattttatatataatatatatatatatatatacacatacattatatatacacaatagtaattctagcaacagaggaaatgacctttaacaggggtataaatctaaatttataaaagtataaatctaaatttcttacccaagacactttaaagatacattatttttctccaggacgtaattcataggaatattaagccttttgtaaatgtccctttagatggtttctcataaggtaaaagaaacttatttccaagcaggaccacctttattgtgtccccagatcacctcacagggcagaaaaatgcccctcagtctgggagaagacctagagagaattatggactccttactggtttttggaaagcaaccaacagctaattccaacaccatgggcagcccatacagtctctaattatctgagaaaatcaaatgatgctgttacaataattacgctggtacaagttaataaaagtgccatgttacagtcaaacagctatgttgctatctataccattgagggcatagttttaaaaagtagttatgctacctgattgtataaggaacaaaactgagagaaaaaatctaaaaggccgcctatgattgaatggaaagattttttttagttgaatttaaataatgtgacttgggggagcctttacaaagagtctttatacctcccttcagcttcctcattttcccttggattacttttgctcaattaaatatgaatttcct

SEQ ID NO: 19 - protein, 149 aa, Artificial Sequence [sequence omitted]

SEQ ID NO: 20 - DNA, 2277 bp, Artificial Sequence
  Synthetic construct
ggcggggcgcgcgcggcggccgttgagggaccgttggggcgggaggcggcggcggcggcggcgcgcgctgcgggcagtgagtgtggaggcgcggacgcgcggcggagctggaactgctgcagctgctgccgccgccggaggaaccttgatccccgtgctccggacaccccgggcctcgccatggctgaccagctgactgaggagcagattgcagagttcaaggaggccttctccctctttgacaaggatggagatggcactatcaccaccaaggagttggggacagtgatgagatccctgggacagaaccccactgaagcagagctgcaggatatgatcaatgaggtggatgcagatgggaacgggaccattgacttcccggagttcctgaccatgatggccagaaagatgaaggacacagacagtgaggaggagatccgagaggcgttccgtgtctttgacaaggatgggaatggctacatcagcgccgcagagctgcgtcacgtaatgacgaacctgggggagaagctgaccgatgaggaggtggatgagatgatcagggaggctgacatcgatggagatggccaggtcaattatgaagagtttgtacagatgatgactgcaaagtgaaggccccccgggcagctggcgatgcccgttctcttgatctctctcttctcgcgcgcgcactctctcttcaacactcccctgcgtaccccggttctagcaaacaccaattgattgactgagaatctgataaagcaacaaaagatttgtcccaagctgcatgattgctctttctccttcttccctgagtctctctccatgcccctcatctcttccttttgccctcgcctcttccatccatgtcttccaaggcctgatgcattcataagttgaagccctccccagatccccttggggagcctctgccctcctccagcccggatggctctcctccattttggtttgtttcctcttgtttgtcatcttattttgggtgctggggtggctgccagccctgtcccgggacctgctgggagggacaagaggccctcccccaggcagaagagcatgccctttgccgttgcatgcaaccagccctgtgattccacgtgcagatcccagcagcctgttggggcaggggtgccaagagaggcattccagaaggactgagggggcgttgaggaattgtggcgttgactggatgtggcccaggagggggtcgagggggccaactcacagaaggggactgacagtgggcaacactcacatcccactggctgctgttctgaaaccatctgattggctttctgaggtttggctgggtggggactgctcatttggccactctgcaaattggacttgcccgcgttcctgaagcgctctcgagctgttctgtaaatacctggtgctaacatcccatgccgctccctcctcacgatgcacccaccgccctgagggcccgtcctaggaatggatgtggggatggtcgctttgtaatgtgctggttctctttttttttctttcccctctatggcccttaagactttcattttgttcagaaccatgctgggctagctaaagggtggggagagggaagatgggccccaccacgctctcaagagaacgcacctgcaataaaacagtcttgtcggccagctgcccaggggacggcagctacagcagcctctgcgtcctggtccgccagcacctcccgcttctccgtggtgacttggcgccgcttcctcacatctgtgctccgtgccctcttccctgcctcttccctcgcccacctgcctgcccccatactcccccagcggagagcatgatccgtgcccttgcttctgactttcgcctctgggacaagtaagtcaatgtgggcagttcagtcgtctgggttttttccccttttctgttcatttcatctggctccccccaccacctccccaccccaccccccaccccctgcttcccctcactgcccaggtcgatcaagtggcttttcctgggacctgcccagctttgagaatctcttctcatccaccctctggcacccagcctctgagggaaggagggatggggcatagtgggagacccagccaagagctgagggtaagggcaggtaggcgtgaggctgtggacattttcggaatgttttggttttgttttttttaaaccgggcaatattgtgttcagttcaagctgtgaagaaaaatatatatcaatgttttccaataaaatacagtgactacctgaaaaaaaaaaaaaaaaaaa

SEQ ID NO: 21 - protein, 164 aa, Artificial Sequence
  Synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 163 aa, Artificial Sequence
  Synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1690 bp, Artificial Sequence
  Synthetic construct
tgctttctcaaaggccccacagtcctccacttcctggggaggtagctgcagaataaaaccagcagagactccttttctcctaaccgtcccggccaccgctgcctcagcctctgcctcccagcctctttctgagggaaaggacaagatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccaggggatttcaccactcaaaggccagacctgcagacgcccagattatgagacacaggatgaagcatttacaacccggttcactcttctcagccactgaagtattcccctttatgtacaggatgctttggttatatttagctccaaaccttcacacacagactgttgtccctgcactctttaagggagtgtactcccagggcttacggccctggccttgggccctctggtttgccggtggtgcaggtagacctgtctcctggcggttcctcgttctccctgggaggcgggcgcactgcctctcacagctgagttgttgagtctgttttgtaaagtccccagagaaagcgcagatgctagcacatgccctaatgtctgtatcactctgtgtctgagtggcttcactcctgctgtaaatttggcttctgttgtcaccttcacctcctttcaaggtaactgtactgggccatgttgtgcctccctggtgagagggccgggcagaggggcagatggaaaggagcctaggccaggtgcaaccagggagctgcaggggcatgggaaggtgggcgggcaggggagggtcagccagggcctgcgagggcagcgggagcctccctgcctcaggcctctgtgccgcaccattgaactgtaccatgtgctacaggggccagaagatgaacagactgaccttgatgagctgtgcacaaagtggcataaaaaacatgtggttacacagtgtgaataaagtgctgcggagcaagaggaggccgttgattcacttcacgctttcagcgaatgacaaaatcatctttgtgaaggcctcgcaggaagacccaacacatgggacctataactgcccagcggacagtggcaggacaggaaaaacccgtcaatgtactaggatactgctgcgtcattacagggcacaggccatggatggaaaacgctctctgctctgctttttttctactgttttaatttatactggcatgctaaagccttcctattttgcataataaatgcttcagtgaaaatgcaaaaaaaaaa

SEQ ID NO: 24 - DNA, 1687 bp, Artificial Sequence
  Synthetic construct
tgctttctcaaaggccccacagtcctccacttcctggggaggtagctgcagaataaaaccagcagagactccttttctcctaaccgtcccggccaccgctgcctcagcctctgcctcccagcctctttctgagggaaaggacaagatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccaggggatttcaccactcaaaggccagacctgcagacgcccagattatgagacacaggatgaagcatttacaacccggttcactcttctcagccactgaagtattcccctttatgtacaggatgctttggttatatttagctccaaaccttcacacacagactgttgtccctgcactctttaagggagtgtactcccagggcttacggccctggccttgggccctctggtttgccggtggtgcaggtagacctgtctcctggcggttcctcgttctccctgggaggcgggcgcactgcctctcacagctgagttgttgagtctgttttgtaaagtccccagagaaagcgcagatgctagcacatgccctaatgtctgtatcactctgtgtctgagtggcttcactcctgctgtaaatttggcttctgttgtcaccttcacctcctttcaaggtaactgtactgggccatgttgtgcctccctggtgagagggccgggcagaggggcagatggaaaggagcctaggccaggtgcaaccagggagctgcaggggcatgggaaggtgggcgggcaggggagggtcagccagggcctgcgagggcagcgggagcctccctgcctcaggcctctgtgccgcaccattgaactgtaccatgtgctacaggggccagaagatgaacagactgaccttgatgagctgtgcacaaagtggcataaaaaacatgtggttacacagtgtgaataaagtgctgcggagcaagaggaggccgttgattcacttcacgctttcagcgaatgacaaaatcatctttgtgaaggcctcgcaggaagacccaacacatgggacctataactgcccagcggacagtggcaggacaggaaaaacccgtcaatgtactaggatactgctgcgtcattacagggcacaggccatggatggaaaacgctctctgctctgctttttttctactgttttaatttatactggcatgctaaagccttcctattttgcataataaatgcttcagtgaaaatgcaaaaaaaaaa

SEQ ID NO: 25 - protein, 482 aa, Artificial Sequence
  Synthetic construct
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 26 - DNA, 2091 bp, Artificial Sequence
  Synthetic construct
gagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttct

SEQ ID NO: 27 - protein, 189 aa, Artificial Sequence
  Synthetic construct
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 28 - DNA, 700 bp, Artificial Sequence
  Synthetic construct
gcccaaggttcagggtcactcaatctcaacagcccagaagcatctgcaacctccccaatggccttgccctttgttttactgatggccctggtggtgctcaactgcaagtcaatctgttctctgggctgtgatctgcctcagacccacagcctgagtaacaggaggactttgatgataatggcacaaatgggaagaatctctcctttctcctgcctgaaggacagacatgactttggatttcctcaggaggagtttgatggcaaccagttccagaaggctcaagccatctctgtcctccatgagatgatccagcagaccttcaatctcttcagcacaaaggactcatctgctacttgggatgagacacttctagacaaattctacactgaactttaccagcagctgaatgacctggaagcctgtatgatgcaggaggttggagtggaagacactcctctgatgaatgtggactctatcctgactgtgagaaaatactttcaaagaatcaccctctatctgacagagaagaaatacagcccttgtgcatgggaggttgtcagagcagaaatcatgagatccttctctttatcagcaaacttgcaagaaagattaaggaggaaggaatgaaaactggttcaacatcgaaatgattctcattgactagtacaccatttcacacttcttgagttctgccgtttca

SEQ ID NO: 29 - protein, 380 aa, Artificial Sequence
  Synthetic construct
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 30 - DNA, 2158 bp, Artificial Sequence
  Synthetic construct
attcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaa

(19) United States
(12) Patent Application Publication
(10) Pub. No.: US 2013/0217656 A1
(43) Pub. Date: Aug. 22, 2013

(54) METHODS AND COMPOSITIONS FOR DIAGNOSING AND TREATING LUPUS

(71) Applicant: George C. Tsokos, Boston, MA (US)

(72) Inventor: George C. Tsokos, Boston, MA (US)

(73) Assignee: Beth Israel Deaconess Medical Center Inc, Boston, MA (US)

(21) Appl. No.: 13/816,676

(22) PCT Filed: Aug. 12, 2011
(86) PCT No.: PCT/US11/47664

§ 371 Date: Apr. 29, 2013

Related U.S. Application Data
(60) Provisional application No. 61/373,185, filed on Aug. 12, 2010.

(51) Int. Cl. C12Q 1/68; G01N 33/68

(52) U.S. Cl. CPC C12Q 1/689; G01N 33/6893
USPC 514/169; 435/6.12; 435/6.11; 435/7.92; 514/630; 506/9

(57) ABSTRACT

The present invention relates to methods, compositions, and diagnostic tests for diagnosing and treating lupus and other related diseases or disease subsets. In particular, the method, compositions, and diagnostic tests relate to a combination of one or more genes, where the expression of these genes indicates a predisposition to develop, or a diagnosis of, lupus and other related diseases or disease subsets.

### CROSS-REFERENCE TO RELATED APPLICATION

This application claims the benefit of the filing date of U.S. Provisional Application No. 61/373,185, filed Aug. 12, 2010, which is hereby incorporated by reference in its entirety.

### STATEMENT AS TO FEDERALLY FUNDED RESEARCH

This invention was made with government support under R01AI42269, R01AI68787, R01AI49954, R01AI85567, and K23AR55672, awarded by the National Institutes of Health. The government has certain rights in the invention.

### BACKGROUND OF THE INVENTION

The present invention relates to methods, compositions, and diagnostic tests for treating lupus and other related diseases or disease subsets.

Lupus manifests in different forms, including systemic lupus erythematosus (SLE). SLE is a clinically heterogeneous disease diagnosed on the presence of a constellation of clinical and laboratory findings. At the pathogenetic level, multiple factors using diverse biochemical and molecular pathways have been recognized. Thus far, recognition and classification of clinical disease subsets of SLE remain difficult, and the availability of specific biomarkers remains at large.

There is an unmet need to accurately identify and classify patients with different clinical manifestations of lupus, which may enable properly targeted treatment. New therapeutic approaches and diagnostic methods are needed to treat lupus and related diseases.

### SUMMARY OF THE INVENTION

The invention is based on the identification of genes and gene combinations that are correlated with patients having or predisposed to developing SLE. We designed a gene expression array (including 38 genes) in order to capture simultaneously using a small amount of blood the levels of each of the genes at a given time point in subjects. The array reported faithfully on the expression levels of each gene, as expected from previous detailed biochemical studies. We performed principal component analysis (PCA) to obtain a better read on the levels of all genes and in doing so we made two exciting observations. First, patients with SLE could be distinguished from normal patients and patients with rheumatoid arthritis (RA), as determined by spatially distinct principal components (i.e., principal components 1, 2, and 3). Second, clinical manifestations (proteinuria and arthritis) were best defined by distinct principal components. Based on this data, we observed that principal components defined patients with SLE apart from normal subjects and that distinct principal components could define clinical manifestations. We believe that this study and approach opens the way for the development of a new tool in identifying patients with SLE and provides a first glimpse in the possibility that the clinical heterogeneity of SLE may be defined along biochemical lines. Our gene expression array should facilitate the diagnosis of SLE with improved sensitivity and specificity, and, when larger cohorts of patients have been studied, it could enable a molecular classification of patients that better dictate treatment.

In particular, we categorized gene expression values into functions (“principal components”) that better represent the variation between individuals. Each determined principal component is a linear combination of expression values, as described herein. One or more principal components correlated with disease, including SLE, arthritis, or proteinuria. Thus, the invention includes methods of diagnosing a patient comprising determining a level of one or more genes in a sample (e.g., a blood sample) and comparing the level to one or more principal components.

The invention also includes methods of treating a subject having SLE that includes this diagnosing step.

Accordingly, the invention features methods, compositions, and diagnostic tests for diagnosing and treating lupus and other related diseases. As there are no tests to accurately diagnose and classify patients with this heterogeneous disease, analysis of expression levels, particularly of the genes described herein, may be used as a novel diagnostic test to identify patients with the disease or disease subset and to treat patients based on this identification. These tests can include any useful metric (e.g., PC 1), as defined herein.

In one aspect, the invention features a method for diagnosing lupus, determining the likelihood of developing lupus, or determining the severity of lupus in a subject, the method including determining an expression level of one or more (e.g., more than one, more than two, more than three, more than five, more than six, more than seven, more than eight, more than nine, more than ten, more than twelve, more than fifteen, more than twenty, more than twenty-five, or more than thirty) genes (e.g., including gene products, as described herein) in a biological sample from the subject, where an increased or a decreased level (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase by more than about 10%, about 15%, about 20%, about 50%, about 75%, about 100%, or about 200%; a decrease by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more) for the one or more genes in the biological sample, as compared to a control (e.g., a control sample from a subject that does not have lupus), is indicative of the presence of lupus, an increased likelihood of developing lupus, or an increased severity of lupus; and where the genes are selected from the group consisting of: interferon alpha 1 (IFNA1); CD247 molecule (CD3ζ) (CD247); cAMP responsive element modulator (CREM); histone deacetylase 1 (HDAC1); nuclear factor of activated T cells, cytoplasmic, calcineurin-dependent 2 (NFATC2); prostaglandin-endoperoxide synthase 2 (prostaglandin G/H synthase and cyclooxygenase) (PTGS2); interferon alpha 5 (IFNA5); cytotoxic T-lymphocyte-associated protein 4 (CTLA4); intercellular adhesion molecule 1 (CD54), human rhinovirus receptor (ICAM1); programmed cell death 1 (PDCD1); rho-associated, coiled-coil containing protein kinase 1 (ROCK1); interleukin 10 (IL10); CD40 ligand (TNF superfamily, member 5, hyper-IgM syndrome) (CD40LG); Fas ligand (TNF superfamily member 6) (FASLG); interferon gamma (IFNG); protein phosphatase 2 (formerly 2A), catalytic subunit, alpha isoform (PPP2CA); spleen tyrosine kinase (SYK); interleukin 23, alpha subunit p19 (IL23A); CD44 molecule (Indian blood group) (CD44); Fc fragment of IgE, high affinity 1, receptor for gamma polypeptide (FCER1G); interleukin 17A (IL17A); protein phosphatase 2 (formerly 2A), catalytic subunit, beta isoform (PPP2CB); ezrin (EZR); v3 variant of CD44 (CD44V3); V-fos FBJ murine osteosarcoma viral oncogene homolog (FOS); interleukin 17F (IL17F); protein kinase, cAMP-dependent, regulatory, type I, beta (PRKAR1B); v6 variant of CD44 (CD44V6); Forkhead box P3 (FOXP3); interleukin 2 (IL2); protein kinase, cAMP-dependent, regulatory, type II, beta (PRKAR2B); CD70 molecule (CD70); GATA binding protein 3 (GATA3); interleukin 21 (IL21); Protein kinase C, delta (PRKCD); calmodulin 3 (phosphorylase kinase, delta) (CALM3); cAMP response element binding protein 1 (CREB1); V-rel reticuloendotheliosis viral oncogene homolog A, nuclear factor of kappa light polypeptide gene enhancer in B-cells, p65 (avian) (RELA); interleukin 6 (IL6); and protein kinase C, theta (PRKCQ).

In some embodiments, the method further includes contacting the biological sample with one or more binding agents capable of specifically binding the one or more genes or the protein encoded by the one or more genes. In some embodiments, the method further includes, prior to determining the expression level, extracting mRNA from the sample (e.g., including one or more of T cells or total peripheral blood mononuclear cells) and reverse transcribing the mRNA into cDNA to obtain a treated biological sample. In particular embodiments, the method further includes contacting the treated biological sample with one or more binding agents capable of specifically binding the one or more genes or the protein encoded by the one or more genes.

In some embodiments, the expression level is determined by one or more of a hybridization assay, an amplification-based assay, or fluorescence in situ hybridization.

In another aspect, the invention features a method for treating lupus in a subject, the method including: administering to the subject a therapeutically effective amount of a therapeutic agent; and determining an expression level of one or more (e.g., more than one, more than two, more than three, more than five, more than six, more than seven, more than eight, more than nine, more than ten, more than twelve, more than fifteen, more than twenty, more than twenty-five, or more than thirty) genes in a biological sample from the subject, where an increased or a decreased level (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase by more than about 10%, about 15%, about 20%, about 50%, about 75%, about 100%, or about 200%; a decrease by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more) for the one or more genes in the biological sample, as compared to a control, is indicative of an increased severity of lupus, thereby indicating administration of an increased dosage of the therapeutic agent or administration of a different therapeutic agent to treat the subject; and where the genes are selected from the group consisting of: IFNA1; CD247; CREM; HDAC1; NFATC2; PTGS2; IFNA5; CTLA4; ICAM1; PDCD1; ROCK1; IL10; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB1; RELA; IL6; and PRKCQ.

In some embodiments, the therapeutic agent is acetaminophen, a nonsteroidal anti-inflammatory drug (e.g., aspirin, naproxen sodium, or ibuprofen), a corticosteroid (e.g., prednisolone), an antimalarial (e.g., hydroxychloroquine), or an immunosuppressant (e.g., azathioprine, cyclophosphamide, methotrexate, mycophenolate, belimumab, rituximab, epratuzumab, abetimus sodium, abatacept, or BG9588 (an anti-CD40L antibody)).

In one aspect, the invention features a method for diagnosing lupus, determining the likelihood of developing lupus, or determining the severity of lupus in a subject, the method including: contacting a biological sample from the subject with one or more (e.g., more than one, more than two, more than three, more than five, more than six, more than seven, more than eight, more than nine, more than ten, more than twelve, more than fifteen, more than twenty, more than twenty-five, or more than thirty) binding agents capable of specifically binding one or more (e.g., more than one, more than two, more than three, more than five, more than six, more than seven, more than eight, more than nine, more than ten, more than twelve, more than fifteen, more than twenty, more than twenty-five, or more than thirty) genes or a protein of one or more (e.g., more than two, more than three, more than five, more than six, more than seven, more than eight, more than nine, more than ten, more than twelve, more than fifteen, more than twenty, more than twenty-five, or more than thirty) genes; and determining an expression level of the one or more genes in the biological sample, where an increased or a decreased level (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase by more than about 10%, about 15%, about 20%, about 50%, about 75%, about 100%, or about 200%; a decrease by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more) for the one or more genes in the biological sample, as compared to a control, is indicative of the presence of lupus, an increased likelihood of developing lupus, or increased severity of lupus; and where the genes are selected from the group consisting of: IFNA1; CD247; CREM; HDAC1; NFATC2; PTGS2; IFNA5; CTLA4; ICAM1; PDCD1; ROCK1; IL10; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB1; RELA; IL6; and PRKCQ.

In another aspect, the invention features a kit for diagnosing a subject having, or having a predisposition to develop, lupus, the kit including: one or more (e.g., more than one, more than two, more than three, more than five, more than six, more than seven, more than eight, more than nine, more than ten, more than twelve, more than fifteen, more than twenty, more than twenty-five, or more than thirty) binding agents capable of specifically binding one or more (e.g., more than one, more than two, more than three, more than five, more than six, more than seven, more than eight, more than nine, more than ten, more than twelve, more than fifteen, more than twenty, more than twenty-five, or more than thirty) genes or a protein encoded by one or more (e.g., more than one, more than two, more than three, more than five, more than six, more than seven, more than eight, more than nine, more than ten, more than twelve, more than fifteen, more than twenty, more than twenty-five, or more than thirty) genes; and instructions for use of the kit, where the genes are selected from the group consisting of: IFNA1; CD247; CREM; HDAC1; NFATC2; PTGS2; IFNA5; CTLA4; ICAM1; PDCD1; ROCK1; IL10; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB1; RELA; IL6; and PRKCQ.

In some embodiments, the one or more binding agents are polynucleotides or polypeptides. In particular embodiments, the one or more binding agents are polynucleotides, and each of the polynucleotides includes a sequence that is substantially identical (e.g., at least 50%, 60%, 70%, 75%, 80%, 85%, 90%, 95%, 96%, 97%, 98%, 99%, or 100% identity) to the sequence of any one of SEQ ID NOs: 2, 11-18, 20, 23, 24, 26, 28, or 30, or a fragment thereof. In other embodiments, the one or more binding agents are polynucleotides, and each of the polynucleotides includes a sequence that is substantially identical (e.g., at least 50%, 60%, 70%, 75%, 80%, 85%, 90%, 95%, 96%, 97%, 98%, 99%, or 100% identity) to a sequence that is substantially complementary (e.g., at least 50%, 60%, 70%, 75%, 80%, 85%, 90%, 95%, 96%, 97%, 98%, 99%, or 100% complementarity) to the sequence of any one of SEQ ID NOs: 2, 11-18, 20, 23, 24, 26, 28, or 30, or a fragment thereof.

In some embodiments, the one or more binding agents are provided on a solid support (e.g., a well, a plate, a wellplate, a tube, an array, a bead, a disc, a microarray, or a microplate, e.g., a microarray).

In other embodiments, the instructions include one or more metrics for a principal component analysis that indicates a diagnosis for lupus or a predisposition to develop lupus.

In any of the aspects and embodiments described herein, the methods, compositions, and diagnostic kits can be used to diagnose and/or treat lupus.

Examples of lupus that can be diagnosed and/or treated according to the present invention include systemic lupus erythematosus, complement deficiency syndrome, cutaneous lupus erythematosus (e.g., chronic cutaneous lupus erythematosus, discoid lupus erythematosus, chilblain lupus erythematosus (Hutchinson), lupus erythematosus-lichen planus overlap syndrome, lupus erythematosus panniculitis (lupus erythematosus profundus), subacute cutaneous lupus erythematosus, tumid lupus erythematosus, and verrucous lupus erythematosus (hypertrophic lupus erythematosus)), drug-induced lupus erythematosus, and neonatal lupus. Diseases related to lupus include other systemic autoimmune diseases (e.g., systemic scleroderma, autoimmune myositis, and vasculitis, including Wegener's granulomatosis) or other diseases generally mistaken for lupus (e.g., rheumatoid arthritis, proteinuria, blood disorders, diabetes, fibromyalgia, Lyme disease, and thyroid disease).

In any of the aspects and embodiments described herein, the expression level is mRNA expression level, cDNA expression level, or protein expression level.

In any of the aspects and embodiments described herein, the expression level is increased (e.g., an increase by about 5%, about 10%, about 15%, about 20%, about 25%, about 30%, about 35%, about 4%,about 45%, about 50%, about 55%, about 60%, about 65%, about 70%, about 75%, about 80%, about 85%, about 90%, about 95%, about 100%, about 150%, about 200%, about 300%, about 400%, about 500%, about 1000%, or more; or an increase by more than about 10%, about 15%, about 20%, about 50%, about 75%, about 100%, about 200%, about 300%, about 400%, about 500%, about 1000%, or more, as compared to a control). In some embodiments, the expression level is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, as compared to a control).

In any of the aspects and embodiments described herein, the expression level is decreased (e.g., a decrease by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, about 300%, about 400%, about 500%, about 1000%, or more; or a decrease by more than about 10%, about 15%, about 20%, about 50%, about 75%, about 100%, about 200%, about 300%, about 400%, about 500%, about 1000%, or more, as compared to a control). In some embodiments, the expression level is decreased (e.g., by less than about 0.01-fold, about 0.02-fold, about 0.1-fold, about 0.3-fold, about 0.5-fold, about 0.8-fold, or less, as compared to a control).

In any of the aspects and embodiments described herein, the method further includes, prior to contacting the sample, extracting mRNA from the sample and/or reverse transcribing the mRNA into cDNA.

In any of the aspects and embodiments described herein, the biological sample includes mRNA, cDNA, and/or protein from the subject.

In any of the aspects and embodiments described herein, the sample obtained from the patient is selected from tissue, whole blood, blood-derived cells (e.g., one or more of T cells or total peripheral blood mononuclear cells), plasma, serum, and combinations thereof.

In any of the aspects and embodiments described herein, the expression level is determined by one or more of a hybridization assay (e.g., northern analysis, ELISA, immunohistochemical analysis, or western blotting), an amplification-based assay (e.g., PCR, quantitative PCR, or real-time quantitative PCR), or fluorescence in situ hybridization.

In any of the aspects and embodiments described herein, the one or more genes are selected from the group consisting of: interferon alpha 1 (IFNA1, UniGene Hs. 37026, Ref. Seq. Nos. NP_{—}008831.3 and NM_{—}024013.1); CD247 molecule (CD3ζ) (CD247, UniGene Hs. 156445, Ref. Seq. Nos. NP_{—}932170.1, NP_{—}000725.1, NM_{—}198053.2, and NM_{—}000734.3); cAMP responsive element modulator (CREM); histone deacetylase 1 (HDAC1, UniGene Hs. 88556, Ref. Seq. Nos. NP_{—}004955.2 and NM_{—}004964.2); nuclear factor of activated T cells, cytoplasmic, calcineurin-dependent 2 (NFATC2, UniGene Hs. 713650, Ref. Seq. Nos. NP_{—}775114.1 and NM_{—}173091.2); prostaglandin-endoperoxide synthase 2 (prostaglandin G/H synthase and cyclooxygenase) (PTGS2, UniGene Hs. 196384, Ref. Seq. Nos. NP_{—}000954.1 and NM_{—}000963.2); interferon alpha 5 (IFNA5, UniGene Hs. 37113, Ref. Seq. Nos. NP_{—}002160.1 and NM_{—}002169.2); CD3e molecule, epsilon (CD3-TCR complex) (CD3E, UniGene Hs. 3003, Ref. Seq. Nos. NP_{—}000724.1 and NM_{—}000733.3); cytotoxic T-lymphocyte-associated protein 4 (CTLA4, UniGene Hs. 247824, Ref. Seq. Nos. NP_{—}005205.2, NM_{—}005214.3, and NM_{—}001037631.1); intercellular adhesion molecule 1 (CD54), human rhinovirus receptor (ICAM1, UniGene Hs. 643447, Ref. Seq. Nos. NP_{—}000192.2 and NM_{—}000201.2); programmed cell death 1 (PDCD1, UniGene Hs. 158297, Ref. Seq. Nos. NP_{—}005009.2 and NM_{—}005018.2); rho-associated, coiled-coil containing protein kinase 1 (ROCK1, UniGene Hs. 306307, Ref. Seq. Nos. NP_{—}005397.1 and NM_{—}005406.2); interleukin 10 (IL10, UniGene Hs. 193717, Ref. Seq. Nos. NP_{—}000563.1 and NM_{—}000572.2); CD40 ligand (TNF superfamily, member 5, hyper-IgM syndrome) (CD40LG, UniGene Hs. 592244, Ref. Seq. Nos. NP_{—}000065.1 and NM_{—}000074.2); Fas ligand (TNF superfamily member 6) (FASLG, UniGene Hs. 2007, Ref. Seq. Nos. NP_{—}000630.1 and NM_{—}000639.1); interferon gamma (IFNG, UniGene Hs. 856, Ref. Seq. Nos. NP_{—}000610.2 and NM_{—}000619.2); protein phosphatase 2 (formerly 2A), catalytic subunit, alpha isoform (PPP2CA, UniGene Hs. 105818, Ref. Seq. Nos. NP_{—}002706.1 and NM_{—}002715.2); spleen tyrosine kinase (SYK, UniGene Hs. 371720, Ref. Seq. Nos. NP_{—}003168.2, NM_{—}003177.5, NM_{—}001135052.2, NM_{—}001174167.1, and NM_{—}001174168.1); interleukin 23, alpha subunit p19 (IL23A, UniGene Hs. 382212 and 98309, Ref. Seq. Nos. NP_{—}057668.1 and NM_{—}016584.2); CD44 molecule (Indian blood group) (CD44, UniGene Hs. 502328, Ref. Seq. Nos. NP_{—}000601.3 (isoform 1), NP_{—}001001389.1 (isoform 2), NP_{—}001001390.1 (isoform 3), NP_{—}001001391.1 (isoform 4), NP_{—}001001392.1 (isoform 5), NP_{—}001189484.1 (isoform 6), NP_{—}001189485.1 (isoform 7), NP_{—}001189486.1 (isoform 8), NM_{—}000610.3 (variant 1), NM_{—}001001389.1 (variant 2), NM_{—}001001390.1 (variant 3), NM_{—}001001391.1 (variant 4), NM_{—}001001392.1 (variant 5), NM_{—}001202555.1 (variant 6), NM_{—}001202556.1 (variant 7), and NM_{—}001202557.1 (variant 8)); Fc fragment of IgE, high affinity 1, receptor for gamma polypeptide (FCER1G, UniGene Hs. 433300, Ref. Seq. Nos. NP_{—}004097.1 and NM_{—}004106.1); interleukin 17A (IL17A, UniGene Hs. 41724, Ref. Seq. Nos. NP_{—}002181.1 and NM_{—}002190.2); protein phosphatase 2 (formerly 2A), catalytic subunit, beta isoform (PPP2CB, UniGene Hs. 491440, Ref. Seq. Nos. NP_{—}001009552.1 and NM_{—}001009552.1); ezrin (EZR, UniGene Hs. 487027, Ref. Seq. Nos. NP_{—}001104547.1, NM_{—}003379.4, and NM_{—}001111077.1); v3 variant of CD44 (CD44V3, UniGene Hs. 502328, Ref. Seq. No. NP_{—}001001390 and NM_{—}001001390.1); V-fos FBJ murine osteosarcoma viral oncogene homolog (FOS, UniGene Hs. 728079, Ref. Seq. Nos. NP_{—}005243.1 and NM_{—}005252.3); interleukin 17F (IL17F, UniGene Hs. 272295, Ref. Seq. Nos. NP_{—}443104.1 and NM_{—}052872.3); protein kinase, cAMP-dependent, regulatory, type I, beta (PRKAR1B, UniGene Hs. 520851, Ref. Seq. Nos. NP_{—}001158230.1, NM_{—}001164761.1 (variant 1), NM_{—}002735.2 (variant 2), NM_{—}001164758.1 (variant 3), NM_{—}001164759.1 (variant 4), NM_{—}001164760.1 (variant 5), NM_{—}001164762.1 (variant 6)); glyceraldehyde-3-phosphate dehydrogenase (GAPDH, UniGene Hs. 544577, 598320, and 592355); v6 variant of CD44 (CD44V6, UniGene Hs. 502328, Ref. Seq. No. NM_{—}001202555.1); Forkhead box P3 (FOXP3, UniGene Hs. 247700, Ref. Seq. Nos. NP_{—}054728.2, NM_{—}014009.3, and NM_{—}001114377.1); interleukin 2 (IL2, UniGene Hs. 89679, Ref. Seq. Nos. NP_{—}000577.2 and NM_{—}000586.3); protein kinase, cAMP-dependent, regulatory, type II, beta (PRKAR2B, UniGene Hs. 433068, Ref. Seq. Nos. NP_{—}002727.2 and NM_{—}002736.2); CD70 molecule (CD70, UniGene Hs. 501497 and 715224, Ref. Seq. Nos. NP_{—}001243.1 and NM_{—}001252.3); GATA binding protein 3 (GATA3, UniGene Hs. 524134, Ref. Seq. Nos. NP_{—}001002295.1, NM_{—}001002295.1, and NM_{—}002051.2); interleukin 21 (IL21, UniGene Hs. 567559, Ref. Seq. Nos. NP_{—}068575.1 and NM_{—}021803.2); Protein kinase C, delta (PRKCD, UniGene Hs. 155342, Ref. Seq. Nos. NP_{—}006245.2, NM_{—}006254.3, and NM_{—}212539.1); calmodulin 3 (phosphorylase kinase, delta) (CALM3, UniGene Hs. 515487, Ref. Seq. Nos. NP_{—}001734.1 and NM_{—}005184.2); cAMP response element binding protein 1 (CREB1, UniGene Hs. 516646, Ref. Seq. Nos. NP_{—}604391.1, NM_{—}134442.3, and NM_{—}004379.3); V-rel reticuloendotheliosis viral oncogene homolog A, nuclear factor of kappa light polypeptide gene enhancer in B-cells, p65 (avian) (RELA, UniGene Hs. 502875, Ref. Seq. Nos. NP_{—}068810.3, NM_{—}021975.3, and NM_{—}001145138.1); interleukin 6 (IL6, UniGene Hs. 654458, Ref. Seq. Nos. NP_{—}000591.1 and NM_{—}000600.3); and protein kinase C, theta (PRKCQ, UniGene Hs. 498570, Ref. Seq. Nos. NP_{—}006248.1 and NM_{—}006257.2), where each sequence recited by the Ref. Seq. No. is incorporated herein by reference.

In any of the aspects and embodiments described herein, the methods, compositions, and diagnostic kits include two or more genes. In some embodiments, the methods, compositions, and diagnostic kits include three or more (e.g., four, five, six, seven, eight, nine, ten, eleven, twelve, thirteen, fourteen, fifteen, sixteen, seventeen, eighteen, nineteen, twenty, twenty-five, thirty, or more) genes.

In any of the aspects and embodiments described herein, the methods, compositions, and diagnostic kits include more than one (e.g., more than two, more than three, more than five, more than six, more than seven, more than eight, more than nine, more than ten, more than twelve, more than fifteen, more than twenty, more than twenty-five, or more than thirty) gene.

In any of the aspects and embodiments described herein, the one or more genes include IL10. In some embodiments, the one or more genes are selected from the group consisting of IL10, IFNA5, CD44, CALM3, CD44V3, FOS, CD247, and HDAC1. In some embodiments, the one or more genes consist of IL10, IFNA5, CD44, CALM3, CD44V3, FOS, CD247, and HDAC1. In some embodiments, the expression level of IL10 is increased (e.g., independently, by more than about 5%, about 10%, about 20%, about 50%, about 75%, about 100%, about 200%, about 500%, or about 1000%) in the biological sample, as compared to a control (e.g., a normal control). In some embodiments, the expression level of IL10 is decreased (e.g., by more than about 5%, about 10%, about 20%, about 50%, about 75%, about 100%, about 200%, about 500%, or about 1000%) in the biological sample (e.g., including total peripheral blood mononuclear cells), as compared to a control (e.g., a normal control).

In any of the aspects and embodiments described herein, the one or more genes include IL10 and CD44; IL10 and CALM3; IL10 and CD44V3; IL10, CD44, and CALM3; IL10, CALM3, and CD44v3; IL10, CD44, CALM3, and CD44V3; CD44 and CALM3; CALM3 and CD44V3; CD44, CALM3, and CD44V3; IL10 and CD247; IL10 and HDAC1; CD427 and HDAC1; IL10, CD427, and HDAC1; IL10, CD44, CALM3, CD44V3, CD247, and HDAC1; IFNA5 and IL10; IFNA5 and CD44V3; IFNA5, IL10, and CD44V3; IFNA5, IL10, CD44V3, and FOS; EZR, IL2, and IL6; CREM, PTGS2, FCER1G, EZR, FOS, IL2, and RELA; ICAM1, CD40LG, FASLG, PPP2CB, GATA3, PRKCD, CREB1, and IL6; or NFATC2, CTLA4, CD40LG, PPP2CB, PRKAR1B, and PRKCQ. In some embodiments, the one or more genes consist of IL10 and CD44; IL10 and CALM3; IL10 and CD44V3; IL10, CD44, and CALM3; IL10, CALM3, and CD44v3; IL10, CD44, CALM3, and CD44V3; CD44 and CALM3; CALM3 and CD44V3; CD44, CALM3, and CD44V3; IL10 and CD247; IL10 and HDAC1; CD427 and HDAC1; IL10, CD427, and HDAC1; IL10, CD44, CALM3, CD44V3, CD247, and HDAC1; IFNA5 and IL10; IFNA5 and CD44V3; IFNA5, IL10, and CD44V3; IFNA5, IL0, CD44V3, and FOS; EZR, IL2, and IL6; CREM, PTGS2, FCER1G, EZR, FOS, IL2, and RELA; ICAM1, CD40LG, FASLG, PPP2CB, GATA3, PRKCD, CREB1, and IL6; or NFATC2, CTLA4, CD40LG, PPP2CB, PRKAR1B, and PRKCQ. In some embodiments, the expression level of each gene (e.g., CD44, CALM3, CD44V3, CD247, HDAC1, CREM, PTGS2, FCER1G, EZR, FOS, IL2, RELA, ICAM1, CD40LG, FASLG, PPP2CB, GATA3, PRKCD, CREB1, IL6, NFATC2, CTLA4, CD40LG, or PPP2CB) is increased (e.g., independently, an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, as compared to a control). In some embodiments, the expression level of each gene (e.g., IFNA5, IL10, PRKAR1B, or PRKCQ) is decreased (e.g., independently, a decrease by less than about 0.01-fold, about 0.02-fold, about 0.1-fold, about 0.3-fold, about 0.5-fold, about 0.8-fold, or less, as compared to a control). In any of the aspects and embodiments described herein, the one or more genes include IL10, IFNA5, CD44, CALM3, CD44V3, FOS, CD247, or HDAC1. In some embodiments, the one or more genes consist of IL10, IFNA5, CD44, CALM3, CD44V3, FOS, CD247, and HDAC1.

In any of the aspects and embodiments described herein, the one or more genes consist of IFNA1; CD247; CREM; HDAC1; NFATC2; PTGS2; IFNA5; CTLA4; ICAM1; PDCD1; ROCK1; IL10; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB; RELA; IL6; and PRKCQ.

In any of the aspects and embodiments described herein, the one or more genes include one or more housekeeping genes (e.g., GAPDH or CD3E) or a control (e.g., HGDC).

In any of the aspects and embodiments described herein, the one or more genes include or consist of any combination described herein.

In any of the aspects and embodiments described herein, the one or more binding agents includes a nucleic acid sequence that is substantially identical (e.g., at least 50%, 60%, 70%, 75%, 80%, 85%, 90%, 95%, 96%, 97%, 98%, 99%, or 100% identical) to the sequence of any one of SEQ ID NOs: 2, 11-18, 20, 23, 24, 26, 28, or 30, or a fragment thereof. In some embodiments, the one or more binding agents includes a nucleic acid sequence that is substantially identical (e.g., at least 50%, 60%, 70%, 75%, 80%, 85%, 90%, 95%, 96%, 97%, 98%, 99%, or 100% identical) to a sequence that is substantially complementary (e.g., at least 50%, 60%, 70%, 75%, 80%, 85%, 90%, 95%, 96%, 97%, 98%, 99%, or 100% complementarity) to the sequence of any one of SEQ ID NOs: 2, 11-18, 20, 23, 24, 26, 28, or 30, or a fragment thereof.

In any of the aspects and embodiments described herein, the one or more binding agents includes a polypeptide (e.g., an antibody) that specifically binds to a sequence that is substantially identical (e.g., at least 50%, 60%, 70%, 75%, 80%, 85%, 90%, 95%, 96%, 97%, 98%, 99%, or 100% identical) to the sequence of any one of SEQ ID NOs: 1, 3-10, 19, 21, 22, 25, 27, or 29, or a fragment thereof.

In particular, the diagnostic methods and tests could aid in classifying patients with particular forms or manifestations of a disease or disease subset. Patients with lupus can exhibit different symptoms with varying severity, and these symptoms can change over time. In part, this variability arises as lupus can affect one or more different organs. The methods described herein can be used to identify subjects with lupus by determining the expression profile of any of the genes described herein. Further, the methods described herein can be used to determine whether a subject has lupus or another disease generally mistaken for lupus (e.g., rheumatoid arthritis, proteinuria, blood disorders, diabetes, fibromyalgia, Lyme disease, and thyroid disease).

Also provided herein are methods of treating a patient with lupus and other related diseases. The diagnostic tests disclosed herein can be used to determine an optimal treatment plan for a subject or to determine the efficacy of a treatment plan for a subject. For example, the subject can be treated for a disease and the prognosis of the disease can be determined by the diagnostic test disclosed herein. In particular embodiments, a diagnostic test or method is used to predict the risk a patient will develop lupus (e.g., SLE). A diagnostic test or method can include a screen for gene expression profiles by any useful detection method (e.g., fluorescence, radiation, or chemiluminescence). A diagnostic test can further include one or more binding agents (e.g., one or more of probes, primers, or antibodies) to detect the expression of these genes. In certain embodiments, the diagnostic test includes the use of one or more genes associated with lupus in a diagnostic platform, which can be optionally automated.

Provided herein are general strategies to develop diagnostic tests, which can be used to predict or diagnose lupus, based on the expression profile of any of the genes disclosed herein (e.g., as used in a principal component). These strategies can be used to develop tests that use one or more of these genes, any combination of one or more of these genes, or one or more of these genes in combination with any other genes found to be associated with lupus.

In certain embodiments, the diagnostic methods and tests include the use of genes in principal component 1, as defined and determined herein. In other embodiments, the diagnostic methods and tests include the use of genes in principal components 1 to 5, as defined and determined herein.

Also provided herein are screening methods, where the method includes contacting a candidate compound (e.g., as described herein) with a reference sample (e.g., a sample for a subject that has lupus, a predisposition for having lupus, or a related disease, such as rheumatoid arthritis) and determining an expression level of the one or more genes in the sample, where an increased or a decreased level (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, %, at 200%, about 300%, about 400%, about 500%, or more; a decrease or an increase by more than about 10%, about 15%, about 20%, about 50%, about 75%, about 100%, or about 200%; a decrease by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more) for the one or more genes in the sample, as compared to a control, is indicative of a therapeutic agent capable of treating of lupus, decreasing the likelihood of developing lupus, or decreasing the severity of lupus; and where the genes are selected from the group consisting of IFNA1; CD247; CREM; HDAC1; NFATC2; PTGS2; IFNA5; CTLA4; ICAM1; PDCD1; ROCK1; IL10; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB1; RELA; IL6; and PRKCQ. In some embodiments, the candidate compound results in a decreased level of one or more genes (e.g., CD44, CALM3, CD44V3, CD247, HDAC1, CREM, PTGS2, FCER1G, EZR, FOS, IL2, RELA, ICAM1, CD40LG, FASLG, PPP2CB, GATA3, PRKCD, CREB1, IL6, NFATC2, CTLA4, CD40LG, PPP2CB, PRKAR1B, or PRKCQ, e.g., CD44V3 or FOS). In other embodiments, the candidate compound results in an increased level of one or more genes (e.g., IL10, IFNA1, IFNA5, IL23A, FASLG, PRKAR1B, or PRKCQ).

Also provided herein are methods of distinguishing other related diseases (e.g., rheumatoid arthritis or proteinuria) from lupus. As described herein, rheumatoid arthritis is best defined by principal component 7, proteinuria by principal component 3, and lupus by principal components 2 and 9. Therefore, PCA can be used to distinguish lupus from other disease, as well as to diagnosis other diseases commonly having similar clinical manifestations as lupus. Accordingly, the invention also includes methods of diagnosing a disease related to lupus (e.g., rheumatoid arthritis or proteinuria) by performing any of the methods or using any of the compositions or kits described herein.

Other features and advantages of the invention will be apparent from the following description and the claims.

### DEFINITIONS

As used herein, the term “about” means ±10% of the recited value.

The term “array” or “microarray,” as used herein refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane. The nucleotide sequences can be DNA, RNA, or any permutations or combinations thereof.

By a “binding agent” is meant a polynucleotide sequence or polypeptide sequence capable of specifically binding a target sequence, or a fragment thereof. By “specifically binds” is meant polynucleotide sequence or polypeptide sequence that recognizes and binds a particular target sequence, or a fragment thereof, but that does not substantially recognize and bind other molecules or other target sequences, including fragments thereof, in a sample, for example, a biological sample. In one example, a polynucleotide that specifically binds to an IL10 binds to the mRNA, cDNA, or protein of IL10, or a fragment thereof, but does not bind to other genes, or fragments thereof. In another example, a polypeptide that specifically binds to an IL10 binds to the mRNA, cDNA, or protein of IL10, or a fragment thereof, but does not bind to other genes, or fragments thereof. In another example, specific binding is determined under various conditions of stringency (See, e.g., Wahl et al., Methods Enzymol. 152:399 (1987); Kimmel, Methods Enzymol. 152:507 (1987)). For example, high stringency salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. High stringency temperature conditions will ordinarily include temperatures of at least about 30° C., 37° C., or 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In an alternative embodiment, hybridization will occur at 50° C. or 70° C. in 400 mM NaCl, 40 mM PIPES, and 1 mM EDTA, at pH 6.4, after hybridization for 12-16 hours, followed by washing. Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. Useful variations on these conditions will be readily apparent to those skilled in the art.

By “biological sample” or “sample” is meant a solid or a fluid sample. Biological samples may include cells; polynucleotide, protein, or membrane extracts of cells (e.g., one or more of T cells or total peripheral blood mononuclear cells); or blood or biological fluids including, e.g., ascites fluid or brain fluid (e.g., cerebrospinal fluid (CSF)). Examples of solid biological samples include samples taken from feces, the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of fluid biological samples include samples taken from the blood, serum, CSF, semen, prostate fluid, seminal fluid, urine, saliva, sputum, mucus, bone marrow, lymph, and tears. Samples may be obtained by standard methods including, e.g., venous puncture and surgical biopsy. In certain embodiments, the biological sample is a blood or serum sample.

By “candidate compound” is meant a chemical, either naturally occurring or artificially derived. Candidate compounds may include, for example, peptides, polypeptides, synthetic organic molecules, naturally occurring organic molecules, nucleic acid molecules, peptide nucleic acid molecules, and components and derivatives thereof. Compounds useful in the invention include those described herein in any of their pharmaceutically acceptable forms, including isomers, such as diastereomers and enantiomers, salts, esters, solvates, and polymorphs thereof, as well as racemic mixtures and pure isomers of the compounds described herein.

By a “control” is meant any useful reference used to diagnose lupus. The control can be any sample, standard, standard curve, or level that is used for comparison purposes. The control can be a normal reference sample or a reference standard or level. A “reference sample” can be, for example, a prior sample taken from the same subject; a sample from a normal healthy subject, such as a normal cell or normal tissue; a sample (e.g., a cell or tissue) from a subject not having lupus, a related disease, or a condition to be differentiated from lupus, such as rheumatoid arthritis; a sample from a subject that is diagnosed with a propensity to develop a lupus or a related disease but does not yet show symptoms of the disorder; a sample from a subject that has been treated for a disease associated with lupus; or a sample of a purified gene (e.g., any described herein) at a known normal concentration. By “reference standard or level” is meant a value or number derived from a reference sample. A normal reference standard or level can be a value or number derived from a normal subject who does not have a disease associated with lupus, a related disease, or a condition to be differentiated from lupus, such as rheumatoid arthritis. In preferred embodiments, the reference sample, standard, or level is matched to the sample subject by at least one of the following criteria: age, weight, sex, disease stage, and overall health. A standard curve of levels of a purified gene, e.g., any described herein, within the normal reference range can also be used as a reference.

By “diagnosing” is meant identifying a molecular or pathological state, disease or condition, such as the identification of lupus or to refer to identification of a subject having lupus who may benefit from a particular treatment regimen.

By “expression” is meant the detection of a gene, polynucleotide, or polypeptide by methods known in the art. For example, DNA expression is often detected by Southern blotting or polymerase chain reaction (PCR), and RNA expression is often detected by northern blotting, RT-PCR, gene array technology, or RNAse protection assays. Methods to measure protein expression level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), and flow cytometry, as well as assays based on a property of the protein including, but not limited to, enzymatic activity or interaction with other protein partners.

By “expression profile” is meant one or more expression values determined for a sample.

By “expression level of a gene” is meant a level of a gene or a gene product, such as mRNA, cDNA, or protein, as compared to a control. The control can be any useful reference, as defined herein. By a “decreased level” or an “increased level” of a gene is meant a decrease or increase in gene expression, as compared to a control (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase by more than about 10%, about 15%, about 20%, about 50%, about 75%, about 100%, or about 200%, as compared to a control; a decrease by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). Gene expression can be determined as the level of a protein or a nucleic acid (e.g., mRNA and/or cDNA), which can be detected by standard art known methods such as those described herein (e.g., as determined by PCR).

By “fragment” is meant a portion of a full-length amino acid or nucleic acid sequence (e.g., any sequence described herein). Fragments may include at least 4, 5, 6, 8, 10, 11, 12, 14, 15, 16, 17, 18, 20, 25, 30, 35, 40, 45, or 50 amino acids or nucleic acids of the full length sequence. A fragment may retain at least one of the biological activities of the full length protein.

A “gene,” “target gene,” “target biomarker,” “target sequence,” “target nucleic acid” or “target protein,” as used herein, is a polynucleotide or protein of interest, the detection of which is desired. Generally, a “template,” as used herein, is a polynucleotide that contains the target nucleotide sequence. In some instances, the terms “target sequence,” “template DNA,” “template polynucleotide,” “target nucleic acid,” “target polynucleotide,” and variations thereof, are used interchangeably.

By “metric” is meant a measure. A metric may be used, for example, to compare the levels of a polypeptide or nucleic acid molecule of interest (e.g., any gene expressed herein). Exemplary metrics include, but are not limited to, mathematical formulas or algorithms, such as one or more ratios or one or more principal components. The metric to be used is that which best discriminates between gene expression levels in a subject having lupus (e.g., SLE) and a normal reference subject or a reference subject not having lupus (e.g., a reference subject with rheumatoid arthritis). Depending on the metric that is used, the diagnostic indicator of lupus may be significantly above or below a reference value. The metric can include both increased level of one or more genes to indicate lupus or decreased level of expression of one of more gene to indicate lupus. These levels can be expressed as one or more expression values or as one or more principal components (PC). In particular embodiments, the metric can be one or more PCs (e.g., PC 1, PC 2, PC 3, PC 4, PC 5, PC 6, PC 7, PC 8, PC 9, PC 10, from PC 1 to PC 2, from PC 1 to PC 3, from PC 1 to PC 4, from PC 1 to PC 5, and other any combinations of one or more of PC 1 to PC 10, as determined herein).

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs.

By “principal component” is meant a linear combination of expression values that represents the variation between the individual expression values of a gene. This linear combination can include a dimensionless multiplier, where the multiplier describes more of the variation in a sample than the expression values independently.

By “solid support” is meant a structure capable of storing, binding, or attaching one or more binding agents.

By “subject” is meant a mammal, including, but not limited to, a human or non-human mammal, such as a bovine, equine, canine, ovine, or feline.

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, or 20 contiguous amino acids, more preferably at least 25, 50, 75, 90, 100, 150, 200, 250, 300, or 350 contiguous amino acids, and most preferably the full-length amino acid sequence. For nucleic acids, the length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

By “substantially complementary” or “substantial complement” is meant a polynucleotide sequence that has the exact complementary polynucleotide sequence, as a target nucleic acid, or has a specified percentage or nucleotides that are the exact complement at the corresponding location within the target nucleic acid when the two sequences are optimally aligned. For example, a polynucleotide sequence that is “substantially complementary” to a target nucleic acid sequence or that is a “substantial complement” to a target nucleic acid sequence has at least 50%, 60%, 70%, 75%, 80%, 85%, 90%, 95%, 96%, 97%, 98%, 99%, or 100% complementarity to the target nucleic acid sequence, or a complement thereof.

By “target sequence” is meant a portion of a gene or a gene product, including the mRNA, related cDNA, or protein encoded by the gene.

By “therapeutic agent” is meant any agent that produces a healing, curative, stabilizing, or ameliorative effect.

A “therapeutically effective amount” of a compound may vary according to factors such as the disease state, age, sex, and weight of the individual, and the ability of the compound to elicit a desired response in the individual. A therapeutically effective amount encompasses an amount in which any toxic or detrimental effects of the compound are outweighed by the therapeutically beneficial effects. A therapeutically effective amount also encompasses an amount sufficient to confer benefit, e.g., clinical benefit.

By “treating” or “ameliorating” is meant administering a composition (e.g., a pharmaceutical composition) for therapeutic purposes or administering treatment to a subject already suffering from a condition or disorder to improve the subject's condition or to reduce the likelihood of a condition or disorder. By “treating a condition or disorder” or “ameliorating a condition or disorder” is meant that the condition or disorder and/or the symptoms associated with the condition or disorder are, e.g., alleviated, reduced, cured, or placed in a state of remission. By “reducing the likelihood of” is meant reducing the severity, the frequency, and/or the duration of a disorder (e.g., SLE) or symptoms thereof. Reducing the likelihood of lupus is synonymous with prophylaxis or the chronic treatment of lupus.

Other features and advantages of the invention will be apparent from the following Detailed Description and the claims.

### BRIEF DESCRIPTION OF THE DRAWINGS

FIGS. 1A-1B show that an SLE gene expression array determines faithfully the levels of studied genes. A. CD3 mRNA levels in normal (N) and systemic lupus erythematosus (SLE) T cells. B. CREM mRNA levels in N and SLE T cells.

FIGS. 2A-2C show gene expression in SLE T cells. A. Gene expression values in patients with SLE. B. First 10 principal components for all patients. C. The percent of variation that each of the principal components accounts for.

FIG. 3 shows the variation between individuals represented on the axes of the first 3 principal components. The upper grey shaded conclave (convex hull) is defined by the position of the entries for the normal individuals. The lower gray shaded conclave is defined by the position of the entries of samples from patients with rheumatoid arthritis.

FIGS. 4A-4C show a correlation between individual principal components and clinical manifestations. A. SLEDAI, B. arthritis, and C. proteinuria. Perpendicular lines represent standard errors.

### DETAILED DESCRIPTION

We have discovered that a combination of one or more genes is correlated with a subject having lupus. In particular, we developed a lupus gene expression array consisting of 30 genes and an additional 8 genes, which were included as controls. T cell mRNA was subjected to reverse transcription and PCR, and the gene expression levels were measured. Conventional statistical analysis was performed along with principal component analysis (PCA) to capture the contribution of all genes to disease diagnosis and clinical parameters. Furthermore, we were able to distinguish between a subject having SLE versus a control (e.g., a normal patient) or a subject having another disease or clinical manifestation, such as rheumatoid arthritis (RA) or proteinuria, using a relatively small amount (about 5 mL) of peripheral blood. PCA of gene expression levels placed SLE samples apart from normal and RA samples regardless of disease activity. Individual principal components tended to define specific disease manifestations such as arthritis and proteinuria. Accordingly, the compositions and methods described herein can be useful for treating or diagnosing a disease, e.g., lupus or rheumatoid arthritis, as well as diagnostic tests (e.g., a solid support, such as an array) for performing such methods. Examples of compositions and methods are described in detail below.

### Principal Component Analysis and Combinations of Genes

The present invention relates to the identification of one or more genes that are correlated with lupus, which can include the use of one or more control or housekeeping genes. In particular, principal component analysis can be used to determine which combination of expression levels would be useful in the methods of the invention.

Principal component analysis (PCA) relies on a mathematical algorithm to convert observations (e.g., expression levels) into a set of components, where each component identifies a data set having the highest variability. By using these components, particular characteristics can be identified in a sample (e.g., the probability that the sample has a diagnostic indicator for lupus that may be significantly above or below a reference value). Each component is a linear combination of the original variables, where each component is orthogonal to each other. Accordingly, PCA transforms a matrix of data into a spatially orthogonal set of new variables, or components. The application of PCA for gene expression profiles is further described in Ringner, Nat. Biotechnol. 2008; 26: 303-304, which is incorporated herein by reference. For example, if an individual was initially characterized by an expression level eₙ for “n” number of genes, then a calculated PC would have the form pcₓ=Σcₙeₙ=c₁e₁+c₂e₂+ . . . +cₙ₋₁eₙ₋₁+cₙeₙ, where each cₙ value is a dimensionless multiplier that is calculated such that pcₓ describes more of the variation in the sample than each eₙ.

Generally, determining the principal components include organizing the data into a m×n matrix, calculative the deviation from the mean, determining the covariance matrix and the eigenvectors and eigenvalues of the covariance matrix, and computing the loading for each eigenvector. Any useful program can be used to determine the proper principal components and cₙ values, such as functions ‘princomp’ or ‘prcomp’ that are available by MATLAB® (as described in the chapter titled “Principal Component Analysis (PCA),” document R2011a for Statistics Toolbox™ by MATLAB®, available on www.mathworks.com/help/toolbox/stats/brkgqnt.html#f75476).

For PCA, any useful data can be used to determine meaningful components. In particular embodiments, the data is one or more expression levels of one or more genes described herein (e.g., any combination of genes described herein). Accordingly, any combination of genes can be used in the methods, compositions, and kits described herein, such as a combination of any of the following genes of the invention: interferon alpha 1 (IFNA1); CD247 molecule (CD3ζ) (CD247); cAMP responsive element modulator (CREM); histone deacetylase 1 (HDAC1); nuclear factor of activated T cells, cytoplasmic, calcineurin-dependent 2 (NFATC2); prostaglandin-endoperoxide synthase 2 (prostaglandin G/H synthase and cyclooxygenase) (PTGS2); interferon alpha 5 (IFNA5); CD3e molecule, epsilon (CD3-TCR complex) (CD3E); cytotoxic T-lymphocyte-associated protein 4 (CTLA4); intercellular adhesion molecule 1 (CD54), human rhinovirus receptor (ICAM1); programmed cell death 1 (PDCD1); rho-associated, coiled-coil containing protein kinase 1 (ROCK1); interleukin 10 (IL10); CD40 ligand (TNF superfamily, member 5, hyper-IgM syndrome) (CD40LG); Fas ligand (TNF superfamily member 6) (FASLG); interferon gamma (IFNG); protein phosphatase 2 (formerly 2A), catalytic subunit, alpha isoform (PPP2CA); spleen tyrosine kinase (SYK); interleukin 23, alpha subunit p19 (IL23A); CD44 molecule (Indian blood group) (CD44); Fc fragment of IgE, high affinity 1, receptor for gamma polypeptide (FCER1G); interleukin 17A (IL17A); protein phosphatase 2 (formerly 2A), catalytic subunit, beta isoform (PPP2CB); ezrin (EZR); v3 variant of CD44 (CD44V3); V-fos FBJ murine osteosarcoma viral oncogene homolog (FOS); interleukin 17F (IL17F); protein kinase, cAMP-dependent, regulatory, type I, beta (PRKAR1B); glyceraldehyde-3-phosphate dehydrogenase (GAPDH); v6 variant of CD44 (CD44V6); Forkhead box P3 (FOXP3); interleukin 2 (IL2); protein kinase, cAMP-dependent, regulatory, type II, beta (PRKAR2B); Human Genomic DNA Contamination (HGDC); CD70 molecule (CD70); GATA binding protein 3 (GATA3); interleukin 21 (IL21); Protein kinase C, delta (PRKCD); calmodulin 3 (phosphorylase kinase, delta) (CALM3); cAMP response element binding protein 1 (CREB1); V-rel reticuloendotheliosis viral oncogene homolog A, nuclear factor of kappa light polypeptide gene enhancer in B-cells, p65 (avian) (RELA); interleukin 6 (IL6); and protein kinase C, theta (PRKCQ).

In some embodiments, the combination includes IL10 and one or more genes selected from the group consisting of IFNA1, CD247, CREM, HDAC1, NFATC2, PTGS2, IFNA5, CTLA4, ICAM1, PDCD1, ROCK1, CD40LG, FASLG, IFNG, PPP2CA, SYK, IL23A, CD44, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CALM3, CREB1, RELA, IL6, and PRKCQ.

In some embodiments, the combination includes IL10, CD44, and one or more genes selected from the group consisting of IFNA1, CD247, CREM, HDAC1, NFATC2, PTGS2, IFNA5, CTLA4, ICAM1, PDCD1, ROCK1, CD40LG, FASLG, IFNG, PPP2CA, SYK, IL23A, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CALM3, CREB1, RELA, IL6, and PRKCQ.

In some embodiments, the combination includes IL10, CALM3, and one or more genes selected from the group consisting of IFNA1, CD247, CREM, HDAC1, NFATC2, PTGS2, IFNA5, CTLA4, ICAM1, PDCD1, ROCK1, CD40LG, FASLG, IFNG, PPP2CA, SYK, IL23A, CD44, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CREB1, RELA, IL6, and PRKCQ.

In some embodiments, the combination includes IL10, CD44V3, and one or more genes selected from the group consisting of IFNA1, CD247, CREM, HDAC1, NFATC2, PTGS2, IFNA5, CTLA4, ICAM1, PDCD1, ROCK1, CD40LG, FASLG, IFNG, PPP2CA, SYK, IL23A, CD44, FCER1G, IL17A, PPP2CB, EZR, FOS, IL17F, PRKAR1B, CD44V6, FOXP3, IL2, PRKAR2B, CD70, GATA3, IL21, PRKCD, CALM3, CREB1, RELA, IL6, and PRKCQ.

In some embodiments, the combination includes IL10, CD44, CALM3, and one or more genes selected from the group consisting of IFNA1, CD247, CREM, HDAC1, NFATC2, PTGS2, IFNA5, CTLA4, ICAM1, PDCD1, ROCK1, CD40LG, FASLG, IFNG, PPP2CA, SYK, IL23A, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CREB1, RELA, IL6, and PRKCQ.

In some embodiments, the combination includes IL10, CALM3, CD44V3, and one or more genes selected from the group consisting of IFNA1, CD247, CREM, HDAC1, NFATC2, PTGS2, IFNA5, CTLA4, ICAM1, PDCD1, ROCK1, CD40LG, FASLG, IFNG, PPP2CA, SYK, IL23A, CD44, FCER1G, IL17A, PPP2CB, EZR, FOS, IL17F, PRKAR1B, CD44V6, FOXP3, IL2, PRKAR2B, CD70, GATA3, IL21, PRKCD, CREB1, RELA, IL6, and PRKCQ.

In some embodiments, the combination includes IL10, CD44, CALM3, CD44V3, and one or more genes selected from the group consisting of IFNA1, CD247, CREM, HDAC1, NFATC2, PTGS2, IFNA5, CTLA4, ICAM1, PDCD1, ROCK1, CD40LG, FASLG, IFNG, PPP2CA, SYK, IL23A, FCER1G, IL17A, PPP2CB, EZR, FOS, IL17F, PRKAR1B, CD44V6, FOXP3, IL2, PRKAR2B, CD70, GATA3, IL21, PRKCD, CREB1, RELA, IL6, and PRKCQ.

In some embodiments, the combination includes CD44 and one or more genes selected from the group consisting of IFNA1, CD247, CREM, HDAC1, NFATC2, PTGS2, IFNA5, CTLA4, ICAM1, PDCD1, ROCK1, IL10, CD40LG, FASLG, IFNG, PPP2CA, SYK, IL23A, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CALM3, CREB1, RELA, IL6, and PRKCQ. In some embodiments, the expression level of CD44 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.5-fold) in the biological sample, as compared to a control (e.g., a normal control).

In some embodiments, the combination includes CALM3 and one or more genes selected from the group consisting of IFNA1, CD247, CREM, HDAC1, NFATC2, PTGS2, IFNA5, CTLA4, ICAM1, PDCD1, ROCK1, IL10, CD40LG, FASLG, IFNG, PPP2CA, SYK, IL23A, CD44, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CREB1, RELA, IL6, and PRKCQ. In some embodiments, the expression level of CALM3 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., more than about 1.5-fold) in the biological sample, as compared to a control (e.g., a normal control).

In some embodiments, the combination includes CD44V3 and one or more genes selected from the group consisting of IFNA1, CD247, CREM, HDAC1, NFATC2, PTGS2, IFNA5, CTLA4, ICAM1, PDCD1, ROCK1, IL10, CD40LG, FASLG, IFNG, PPP2CA, SYK, IL23A, CD44, FCER1G, IL17A, PPP2CB, EZR, FOS, IL17F, PRKAR1B, CD44V6, FOXP3, IL2, PRKAR2B, CD70, GATA3, IL21, PRKCD, CALM3, CREB1, RELA, IL6, and PRKCQ. In some embodiments, the expression level of CD44V3 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., more than about 1000-fold, about 1500-fold, or about 2000-fold) in the biological sample, as compared to a control (e.g., a normal control).

In some embodiments, the combination includes CD44, CALM3, and one or more genes selected from the group consisting of IFNA1, CD247, CREM, HDAC1, NFATC2, PTGS2, IFNA5, CTLA4, ICAM1, PDCD1, ROCK1, IL10, CD40LG, FASLG, IFNG, PPP2CA, SYK, IL23A, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CREB1, RELA, IL6, and PRKCQ. In some embodiments, the expression level of CD44 and CALM3 are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, as compared to a control) in the biological sample, as compared to a control (e.g., a normal control).

In some embodiments, the combination includes CALM3, CD44V3, and one or more genes selected from the group consisting of IFNA1, CD247, CREM, HDAC1, NFATC2, PTGS2, IFNA5, CTLA4, ICAM1, PDCD1, ROCK1, IL10, CD40LG, FASLG, IFNG, PPP2CA, SYK, IL23A, CD44, FCER1G, IL17A, PPP2CB, EZR, FOS, IL17F, PRKAR1B, CD44V6, FOXP3, IL2, PRKAR2B, CD70, GATA3, IL21, PRKCD, CREB1, RELA, IL6, and PRKCQ. In some embodiments, the expression level of CALM3 and CD44V3 are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more) in the biological sample, as compared to a control (e.g., a normal control).

In some embodiments, the combination includes CD44, CALM3, CD44V3, and one or more genes selected from the group consisting of IFNA1, CD247, CREM, HDAC1, NFATC2, PTGS2, IFNA5, CTLA4, ICAM1, PDCD1, ROCK1, IL10, CD40LG, FASLG, IFNG, PPP2CA, SYK, IL23A, FCER1G, IL17A, PPP2CB, EZR, FOS, IL17F, PRKAR1B, CD44V6, FOXP3, IL2, PRKAR2B, CD70, GATA3, IL21, PRKCD, CREB1, RELA, IL6, and PRKCQ. In some embodiments, the expression level of CD44, CALM3, and CD44V3 are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more) in the biological sample, as compared to a control (e.g., a normal control).

In some embodiments, the combination includes CD247 and one or more genes selected from the group consisting of IFNA1, CREM, HDAC1, NFATC2, PTGS2, IFNA5, CTLA4, ICAM1, PDCD1, ROCK1, IL10, CD40LG, FASLG, IFNG, PPP2CA, SYK, IL23A, CD44, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CALM3, CREB1, RELA, IL6, and PRKCQ. In some embodiments, the expression level of CD247 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.2 fold) in the biological sample, as compared to a control (e.g., a normal control). In particular embodiments, the combination includes IL10, CD247, and one or more genes provided herein. In yet other embodiments, the combination includes CD247 and one or more genes selected from IL10, CD44, CALM3, CD44V3, and HDAC1.

In some embodiments, the combination includes HDAC1 and one or more genes selected from the group consisting of IFNA1, CD247, CREM, NFATC2, PTGS2, IFNA5, CTLA4, ICAM1, PDCD1, ROCK1, IL10, CD40LG, FASLG, IFNG, PPP2CA, SYK, IL23A, CD44, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CALM3, CREB1, RELA, IL6, and PRKCQ. In some embodiments, the expression level of HDAC1 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.2 fold) in the biological sample, as compared to a control (e.g., a normal control). In particular embodiments, the combination includes IL10, HDAC1, and one or more genes provided herein. In yet other embodiments, the combination includes HDAC1 and one or more genes selected from IL10, CD44, CALM3, CD44V3, and CD247.

In some embodiments, the combination includes CD247, HDAC1, and one or more genes selected from the group consisting of IFNA1, CREM, NFATC2, PTGS2, IFNA5, CTLA4, ICAM1, PDCD1, ROCK1, IL10, CD40LG, FASLG, IFNG, PPP2CA, SYK, IL23A, CD44, FCER1G, IL17A, PPP2CB, EZR, CD44V3, FOS, IL17F, PRKAR1B, CD44V6, FOXP3, IL2, PRKAR2B, CD70, GATA3, IL21, PRKCD, CALM3, CREB1, RELA, IL6, and PRKCQ. In some embodiments, the expression level of CD247 and HDAC1 are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.2 fold) in the biological sample, as compared to a control (e.g., a normal control).

In some embodiments, the combination includes IL10, CD44, CALM3, CD44V3, CD247, HDAC1, and one or more genes selected from the group consisting of IFNA1, CREM, NFATC2, PTGS2, IFNA5, CTLA4, ICAM1, PDCD1, ROCK1, CD40LG, FASLG, IFNG, PPP2CA, SYK, IL23A, FCER1G, IL17A, PPP2CB, EZR, FOS, IL17F, PRKAR1B, CD44V6, FOXP3, IL2, PRKAR2B, CD70, GATA3, IL21, PRKCD, CREB1, RELA, IL6, and PRKCQ. In some embodiments, the expression level of CD44, CALM3, CD44V3, CD247, and HDAC1 are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.2 fold) in the biological sample, as compared to a control (e.g., a normal control).

In some embodiments, the combination includes IFNA5 and one or more genes selected from the group consisting of IFNA1; CD247; CREM; HDAC1; NFATC2; PTGS2; CTLA4; ICAM1; PDCD1; ROCK1; IL10; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB1; RELA; IL6; and PRKCQ. In some embodiments, the expression level of IFNA5 is decreased (e.g., by less than about 0.01-fold, about 0.02-fold, about 0.1-fold, about 0.3-fold, about 0.5-fold, about 0.8-fold, or less, e.g., less than about 0.02-fold, e.g., by about 0.02-fold) in the biological sample (e.g., having total peripheral blood mononuclear cells), as compared to a control (e.g., a normal control).

In some embodiments, the combination includes IFNA5, IL10, and one or more genes selected from the group consisting of IFNA1; CD247; CREM; HDAC1; NFATC2; PTGS2; CTLA4; ICAM1; PDCD1; ROCK1; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB1; RELA; IL6; and PRKCQ. In some embodiments, the expression level of IFNA5 and IL10 are decreased (e.g., independently, by less than about 0.01-fold, about 0.02-fold, about 0.1-fold, about 0.3-fold, about 0.5-fold, about 0.8-fold, or less, e.g., less than about 0.02-fold, e.g., by about 0.02-fold) in the biological sample (e.g., having total peripheral blood mononuclear cells), as compared to a control (e.g., a normal control).

In some embodiments, the combination includes IFNA5, CD44V3, and one or more genes selected from the group consisting of IFNA1; CD247; CREM; HDAC1; NFATC2; PTGS2; CTLA4; ICAM1; PDCD1; ROCK1; IL10; CD40LG; FASLG; IFNG; PPP2CA; SYK; IL23A; CD44; FCER1G; IL17A; PPP2CB; EZR; FOS; IL17F; PRKAR1B; CD44V6; FOXP3; IL2; PRKAR2B; CD70; GATA3; IL21; PRKCD; CALM3; CREB1; RELA; IL6; and PRKCQ. In some embodiments, the expression level of IFNA5 is decreased (e.g., independently, by less than about 0.01-fold, about 0.02-fold, about 0.1-fold, about 0.3-fold, about 0.5-fold, about 0.8-fold, or less, e.g., less than about 0.02-fold, e.g., by about 0.02-fold) in the biological sample (e.g., having total peripheral blood mononuclear cells), as compared to a control (e.g., a normal control). In some embodiments, the expression level of CD44V3 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., more than about 1000-fold, about 1500-fold, or about 2000-fold) in the biological sample (e.g., having total peripheral blood mononuclear cells), as compared to a control (e.g., a normal control).

In some embodiments, the combination includes IFNA5, IL10, CD44V3, and one or more genes selected from the group consisting of IFNA1; CD247; CREM; HDAC1; NFATC2; PTGS2; CTLA4; ICAM1; PDCD1; ROCK1; CD40LG; FASLG; IFNG; PPP2CA; SYK; IL23A; CD44; FCER1G; IL17A; PPP2CB; EZR; FOS; IL17F; PRKAR1B; CD44V6; FOXP3; IL2; PRKAR2B; CD70; GATA3; IL21; PRKCD; CALM3; CREB1; RELA; IL6; and PRKCQ. In some embodiments, the expression level of IFNA5 and IL10 are decreased (e.g., independently, by less than about 0.01-fold, about 0.02-fold, about 0.1-fold, about 0.3-fold, about 0.5-fold, about 0.8-fold, or less, e.g., less than about 0.02-fold, e.g., by about 0.02-fold) in the biological sample (e.g., having total peripheral blood mononuclear cells), as compared to a control (e.g., a normal control). In some embodiments, the expression level of CD44V3 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., more than about 1000-fold, about 1500-fold, or about 2000-fold) in the biological sample (e.g., having total peripheral blood mononuclear cells), as compared to a control (e.g., a normal control).

In some embodiments, the combination includes IFNA5, IL10, CD44V3, FOS, and one or more genes selected from the group consisting of IFNA1; CD247; CREM; HDAC1; NFATC2; PTGS2; CTLA4; ICAM1; PDCD1; ROCK1; CD40LG; FASLG; IFNG; PPP2CA; SYK; IL23A; CD44; FCER1G; IL17A; PPP2CB; EZR; IL17F; PRKAR1B; CD44V6; FOXP3; IL2; PRKAR2B; CD70; GATA3; IL21; PRKCD; CALM3; CREB1; RELA; IL6; and PRKCQ. In some embodiments, the expression level of IFNA5 and IL10 are decreased (e.g., independently, by less than about 0.01-fold, about 0.02-fold, about 0.1-fold, about 0.3-fold, about 0.5-fold, about 0.8-fold, or less, e.g., less than about 0.02-fold, e.g., by about 0.02-fold) in the biological sample (e.g., having total peripheral blood mononuclear cells), as compared to a control (e.g., a normal control). In some embodiments, the expression level of CD44V3 and FOS are increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., more than about 5.0-fold, 10-fold, about 1000-fold, about 1500-fold, or about 2000-fold) in the biological sample (e.g., having total peripheral blood mononuclear cells), as compared to a control (e.g., a normal control).

In some embodiments, the combination includes EZR, IL2, IL6, and one or more genes selected from the group consisting of IFNA1; CD247; CREM; HDAC1; NFATC2; PTGS2; IFNA5; CTLA4; ICAM1; PDCD1; ROCK1; IL10; CD40LG; FASLG; IFNG; PPP2CA; SYK; IL23A; CD44; FCER1G; IL17A; PPP2CB; EZR; CD44V3; FOS; IL17F; PRKAR1B; CD44V6; FOXP3; IL2; PRKAR2B; CD70; GATA3; IL21; PRKCD; CALM3; CREB1; RELA; IL6; and PRKCQ. In some embodiments, the expression level of EZR, IL2, and IL6 are increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., more than about 1.8-fold, about 2.0-fold, about 3.0-fold, about 3.5-fold, about 4.5-fold, or about 5.0-fold, e.g., more than about 3.0-fold) in the biological sample (e.g., having total peripheral blood mononuclear cells), as compared to a control (e.g., a normal control).

In some embodiments, the combination includes CREM, PTGS2, FCER1G, EZR, FOS, IL2, RELA, and one or more genes selected from the group consisting of IFNA1, CD247, HDAC1, NFATC2, IFNA5, CTLA4, ICAM1, PDCD1, ROCK1, IL10, CD40LG, FASLG, IFNG, PPP2CA, SYK, IL23A, CD44, IL17A, PPP2CB, CD44V3, IL17F, PRKAR1B, CD44V6, FOXP3, PRKAR2B, CD70, GATA3, IL21, PRKCD, CALM3, CREB1, IL6, and PRKCQ. In some embodiments, the expression level of CREM, PTGS2, FCER1G, EZR, FOS, IL2, and RELA are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.2 fold) in the biological sample, as compared to a control (e.g., a normal control). In some embodiments, this combination also includes IL10.

In some embodiments, the combination includes ICAM1, CD40LG, FASLG, PPP2CB, GATA3, PRKCD, CREB1, IL6, and one or more genes selected from the group consisting of IFNA1, CD247, CREM, HDAC1, NFATC2, PTGS2, IFNA5, CTLA4, PDCD1, ROCK1, IL10, IFNG, PPP2CA, SYK, IL23A, CD44, FCER1G, IL17A, EZR, CD44V3, FOS, IL17F, PRKAR1B, CD44V6, FOXP3, IL2, PRKAR2B, CD70, IL21, CALM3, RELA, and PRKCQ. In some embodiments, the expression level of ICAM1, CD40LG, FASLG, PPP2CB, GATA3, PRKCD, CREB1, and IL6 are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.2 fold) in the biological sample, as compared to a control (e.g., a normal control). In some embodiments, this combination also includes IL10.

In some embodiments, the combination includes NFATC2, CTLA4, CD40LG, PPP2CB, PRKAR1B, PRKCQ, and one or more genes selected from the group consisting of IFNA1, CD247, CREM, HDAC1, PTGS2, IFNA5, ICAM1, PDCD1, ROCK1, IL10, FASLG, IFNG, PPP2CA, SYK, IL23A, CD44, FCER1G, IL17A, EZR, CD44V3, FOS, IL17F, CD44V6, FOXP3, IL2, PRKAR2B, CD70, GATA3, IL21, PRKCD, CALM3, CREB1, RELA, and IL6. In some embodiments, the expression level of NFATC2, CTLA4, CD40LG, PPP2CB, PRKAR1B, and PRKCQ are increased (e.g., independently, by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., by more than about 1.2 fold) in the biological sample, as compared to a control (e.g., a normal control). In some embodiments, the expression level of PRKAR1B and PRKCQ are decreased (e.g., independently, by less than about 0.01-fold, about 0.02-fold, about 0.1-fold, about 0.3-fold, about 0.5-fold, about 0.8-fold, or less, e.g., less than about 0.8-fold) in the biological sample, as compared to a control (e.g., a normal control). In some embodiments, this combination also includes IL10.

In any of the above embodiments, the expression level of IL10 is decreased (e.g., by less than about 0.01-fold, about 0.02-fold, about 0.1-fold, about 0.3-fold, about 0.5-fold, about 0.8-fold, or less, e.g., less than about 0.02-fold, e.g., by about 0.02-fold) in the biological sample (e.g., having total peripheral blood mononuclear cells), as compared to a control (e.g., a normal control).

In any of the above embodiments, the expression level of IFNA5 is decreased (e.g., by less than about 0.01-fold, about 0.02-fold, about 0.1-fold, about 0.3-fold, about 0.5-fold, about 0.8-fold, or less, e.g., less than about 0.02-fold, e.g., by about 0.02-fold) in the biological sample (e.g., having total peripheral blood mononuclear cells), as compared to a control (e.g., a normal control).

In any of the above embodiments, the expression level of CD44V3 is increased (e.g., by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., more than about 1000-fold, about 1500-fold, or about 2000-fold) in the biological sample, as compared to a control (e.g., a normal control).

In some embodiments of any combination described above, the combination includes one or more housekeeping genes selected from GAPDH, HGDC, CD3E, EZR, FOXP3, ICAM1, PTGS2, and ROCK1.

### Diagnostic Methods

The present invention features methods and compositions to diagnose lupus and monitor the progression of such a disorder. For example, the methods can include determining an expression level of one or more genes in a biological sample and comparing the level to a normal reference. The expression level of a gene, e.g., any described herein, can be determined by one or more of mRNA expression level, cDNA expression level, or protein expression level. These genes and their gene products can also be used to monitor the therapeutic efficacy of compounds, including therapeutic agents described herein, used to treat lupus or a related disorder (e.g., RA).

Alterations in the expression or biological activity of one or more genes of the invention in a test sample as compared to a normal reference can be used to diagnose lupus or a related disease (e.g., RA).

Expression of various genes or biomarkers in a sample can be analyzed by a number of methodologies, many of which are known in the art and understood by the skilled artisan, including but not limited to, immunohistochemical and/or western blot analysis, immunoprecipitation, molecular binding assays, ELISA, ELIFA, fluorescence activated cell sorting (FACS) and the like, quantitative blood based assays (as for example serum ELISA) (to examine, for example, levels of protein expression), biochemical enzymatic activity assays, in situ hybridization, northern analysis and/or PCR analysis of mRNAs, as well as any one of the wide variety of assays that can be performed by gene and/or tissue array analysis. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting), and 18 (PCR Analysis). Multiplexed immunoassays such as those available from Rules Based Medicine or Meso Scale Discovery (MSD) may also be used.

A sample comprising a target gene or biomarker can be obtained by methods well known in the art. For instance, samples from a subject may be obtained by venipuncture, resection, bronchoscopy, fine needle aspiration, bronchial brushings, or from sputum, pleural fluid, or blood, such as serum or plasma. Genes or gene products (e.g., mRNA, cDNA, or protein) can be detected from these samples. By screening such body samples, a simple early diagnosis can be achieved for lupus or related diseases. In addition, the progress of therapy can be monitored more easily by testing such body samples for target genes or gene products.

In certain embodiments, the expression a protein of one or more genes in a sample is examined using immunohistochemistry (“IHC”) and staining protocols. IHC staining of tissue sections has been shown to be a reliable method of assessing or detecting presence of proteins in a sample. IHC techniques use an antibody to probe and visualize cellular antigens in situ, generally by chromogenic or fluorescent methods. The tissue sample may be fixed (i.e., preserved) by conventional methodology (see, e.g., “Manual of Histological Staining Method of the Armed Forces Institute of Pathology,” 3^{rd} edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. By way of example, neutral buffered formalin, Bouin's or paraformaldehyde, may be used to fix a sample. Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. The primary and/or secondary antibody used for immunohistochemistry typically will be labeled with a detectable moiety, such as a radioisotope, a colloidal gold particle, a fluorescent label, a chromogenic label, or an enzyme-substrate label.

In alternative methods, the sample may be contacted with an antibody specific for the gene or biomarker under conditions sufficient for an antibody-biomarker complex to form, and then detecting the complex. The presence of the biomarker may be detected in a number of ways, such as by western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the noncompetitive types, as well as in the traditional competitive binding assays. These assays also include direct binding of a labeled antibody to a target biomarker.

Another method involves immobilizing the target biomarkers (e.g., on a solid support) and then exposing the immobilized target to specific antibody which may or may not contain a label. Depending on the amount of target and the strength of the label's signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by a label, e.g., an enzyme, a fluorescent label, a chromogenic label, a radionuclide containing molecule (i.e., a radioisotope), and a chemiluminescent molecule.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface (e.g., a glass or a polymer surface, such as those with solid supports in the form of tubes, beads, discs, or microplates), and a second antibody is linked to a label that is used to indicate the binding of the second antibody to the molecular marker.

Another methodology for determining expression level in a sample is in situ hybridization, for example, fluorescence in situ hybridization (FISH) (see, e.g., Angerer et al., Methods Enzymol. 152:649-661, 1987). Generally, in situ hybridization includes the following steps: (1) fixation of a biological sample to be analyzed; (2) pre-hybridization treatment of the biological sample to increase accessibility of target DNA and to reduce non-specific binding; (3) hybridization of the mixture of nucleic acids to the nucleic acid in the biological sample; (4) post-hybridization washes to remove nucleic acid fragments not bound in the hybridization; and (5) detection of the hybridized nucleic acid fragments. The binding agents (e.g., probes) used in such applications are typically labeled, for example, with radioisotopes or fluorescent labels. Preferred probes are sufficiently long, for example, from about 50, 100, or 200 nucleotides to about 1000 or more nucleotides, to enable specific hybridization with the target nucleic acid(s) under stringent conditions.

Amplification-based assays also can be used to measure the expression level of one or more genes. In such assays, the nucleic acid sequences of the gene act as a template in an amplification reaction (for example, a polymerase chain reaction (PCR) or quantitative PCR). In a quantitative amplification, the amount of amplification product will be proportional to the amount of template in the original sample. Comparison to appropriate controls provides a measure of the expression level of the gene, corresponding to the specific probe used, according to the principles discussed above. Methods of real-time quantitative PCR using TaqMan probes are well known in the art. Detailed protocols for real-time quantitative PCR are provided, for example, in Gibson et al., Genome Res. 6:995-1001, 1996, and in Heid et al., Genome Res. 6:986-994, 1996.

Based on the sequences of the genes provided herein, one of skill in the art would be able to use these sequences to design and construct primers that can specifically bind to the mRNA or cDNA sequence in order to perform an amplification-based assay. Any useful program can be used to design primers, such as Primer Premier (available by Premier Biosoft International, Palo Alto, Calif.), Primer-Blast (available at www.ncbi.nlm.nih.gov/tools/primer-blast/ by NCBI), Primer3 (available at biotools.umassmed.edu/bioapps/primer3_www.cgi), and OligoAnalyzer (available at www.idtdna.com/SciTools/SciTools.aspx by Integrated DNA Technologies, Inc., San Diego, Calif.).

A TaqMan-based assay also can be used to quantify expression level. TaqMan-based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification.

Other suitable amplification methods include, but are not limited to, ligase chain reaction (LCR) (see, e.g., Wu and Wallace, Genomics 4:560-569, 1989; Landegren et al., Science 241: 1077-1080, 1988; and Barringer et al., Gene 89:117-122, 1990), transcription amplification (see, e.g., Kwoh et al., Proc. Natl. Acad. Sci. USA 86:1173-1177, 1989), self-sustained sequence replication (see, e.g., Guatelli et al., Proc. Natl. Acad. Sci. USA 87:1874-1878, 1990), dot PCR, and linker adapter PCR.

Expression levels may also be determined using microarray-based platforms (e.g., single-nucleotide polymorphism (SNP) arrays), as microarray technology offers high resolution. Details of various microarray methods can be found in the literature. See, for example, U.S. Pat. No. 6,232,068 and Pollack et al., Nat. Genet. 23:41-46, 1999.

Methods of the invention further include protocols which examine the presence and/or expression of mRNAs of one or more genes, in a tissue or cell sample. Methods for the evaluation of mRNAs in cells are well known and include, for example, hybridization assays using complementary DNA probes (such as in situ hybridization using labeled riboprobes specific for the one or more genes, northern blot and related techniques) and various nucleic acid amplification assays (such as RT-PCR using complementary primers specific for one or more of the genes, and other amplification type detection methods, such as, for example, branched DNA, SISBA, TMA, and the like).

Tissue or cell samples from mammals can be conveniently assayed for mRNAs using northern, dot blot or PCR analysis. For example, RT-PCR assays such as quantitative PCR assays are well known in the art. In an illustrative embodiment of the invention, a method for detecting a target mRNA in a biological sample comprises producing cDNA from the sample by reverse transcription using at least one primer; amplifying the cDNA so produced using a target polynucleotide as sense and antisense primers to amplify target cDNAs therein; and detecting the presence of the amplified target cDNA using polynucleotide probes. In some embodiments, primers and probes comprising the sequences described herein are used to detect expression of one or more genes, as described herein. In addition, such methods can include one or more steps that allow one to determine the levels of target mRNA in a biological sample (e.g., by simultaneously examining the levels a comparative control mRNA sequence of a “housekeeping” gene such as an actin family member or any control gene described herein, such as GAPDH). Optionally, the sequence of the amplified target cDNA can be determined.

Optional methods of the invention include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes can then hybridized to an array of nucleic acids immobilized on a solid support. The array can be configured such that the sequence and position of each member of the array is known. For example, a selection of genes whose expression correlate with the presence of lupus, an increased likelihood of developing lupus, or increased severity of lupus be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment, (see, e.g., WO 01/75166 published Oct. 11, 2001; (see, for example, U.S. Pat. No. 5,700,637, U.S. Pat. No. 5,445,934, and U.S. Pat. No. 5,807,522, Lockart, Nat. Biotechnol. 14:1675-1680 (1996); Cheung et al., Nat. Genet. 21(Suppl):15-19 (1999) for a discussion of array fabrication).

DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). Commercially available microarray systems can be used, such as the Affymetrix GeneChip® system.

Expression of a selected gene or biomarker in a tissue or cell sample may also be examined by way of functional or activity-based assays. For instance, if the biomarker is an enzyme, one may conduct assays known in the art to determine or detect the presence of the given enzymatic activity in the tissue or cell sample.

Any of the methods herein can be adapted to include a solid support. Exemplary solid supports include a glass or a polymer surface, including one or more of a well, a plate, a wellplate, a tube, an array, a bead, a disc, a microarray, or a microplate. In particular, the solid supported can be adapted to allow for automation of any one of the methods described herein (e.g., PCR).

Detection of amplification, overexpression, or overproduction of, for example, a gene or gene product can also be used to provide prognostic information or guide therapeutic treatment. Such prognostic or predictive assays can be used to determine prophylactic treatment of a subject prior to the onset of symptoms of, e.g., lupus or a related disease (e.g., RA).

The diagnostic methods described herein can be used individually or in combination with any other diagnostic method described herein for a more accurate diagnosis of the presence or severity of a disorder (e.g., lupus or a related disorder). Examples of additional methods for diagnosing such disorders include, e.g., examining a subject's health history, immunohistochemical staining of tissues, or performing one or more laboratory tests, such as anti-DNA antibody detection, level of erythrocyte sedimentation rate, level of C-reactive protein, antinuclear antibody detection, level of complement values (e.g., C3 and C4), antiphospholipid antibody detection, or level of creatinine clearance.

Binding Agent

A binding agent that specifically binds a target gene or a gene product (e.g., mRNA, cDNA, or protein) may be used for the diagnosis of a disease, such as lupus. The binding agent may be, e.g., a protein (e.g., an antibody, antigen, or fragment thereof) or a polynucleotide. The polynucleotide may possess sequence specificity for the gene (e.g., as in a primer) or may be an aptamer.

Based on genes and sequences (e.g., any one of SEQ ID NOs: 1-30) provided herein, one of skill in the art would be able to use these sequences to design and construct binding agents that can specifically bind to the mRNA, cDNA, or protein sequence. For example, the particular sequence for a gene is provided in the UniGene database, where accession numbers for each gene is provided herein. Any useful program can be used to input a sequence and design primers, such as Primer Premier (available by Premier Biosoft International, Palo Alto, Calif.), Primer-Blast (available at www.ncbi.nlm.nih.gov/tools/primer-blast/ by NCBI), Primer3 (available at biotools.umassmed.edu/bioapps/primer3_www.cgi), and OligoAnalyzer (available at www.idtdna.com/SciTools/SciTools.aspx by Integrated DNA Technologies, Inc., San Diego, Calif.).

Preferably, each binding agent specifically binds to a particular gene or gene product (e.g., mRNA, cDNA, or protein). For determining an expression level of a protein, the measurement of antibodies specific to a polypeptide of the invention (i.e., a protein product of any of the genes of the invention, such as described herein) in a subject may be used for the diagnosis of lupus or a propensity to develop the same. Antibodies specific to one or more polypeptides of the invention (e.g., one or more of SEQ ID NOs: 1, 3-10, 19, 21, 22, or 25, or a particular sequence for a protein provided in the UniGene database, where accession numbers for each gene is provided herein) may be measured in any bodily fluid, including, but not limited to, urine, blood, serum, plasma, saliva, or cerebrospinal fluid. ELISA assays are the preferred method for measuring levels of antibodies in a bodily fluid.

For determining an expression level of mRNA or cDNA, polynucleotides that hybridize to a gene of the invention at high stringency may be used as a probe to monitor expression levels. Methods for detecting such levels are standard in the art and are described in Sandri et al. (Cell, 117:399-412, 2004). In one example, northern blotting or real-time PCR is used to detect mRNA levels (Sandri et al., supra, and Bdolah et al., Am. J. Physio. Regul. Integre. Comp. Physiol. 292:R971-R976, 2007). Binding can be determined at various stringency conditions, such as at high stringency conditions. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences.

The binding agent may optionally contain a label, such as a radioisotope, a colloidal gold particle, a fluorescent label, a chromogenic label, an enzyme-substrate label, or a chemiluminescent label.

### Methods of Treatment

The methods, compositions, and diagnostic tests can be used to treat or diagnose lupus or a related disease (e.g., RA). Lupus includes all different forms, including systemic lupus erythematosus, complement deficiency syndrome, cutaneous lupus erythematosus (e.g., chronic cutaneous lupus erythematosus, discoid lupus erythematosus, chilblain lupus erythematosus (Hutchinson), lupus erythematosus-lichen planus overlap syndrome, lupus erythematosus panniculitis (lupus erythematosus profundus), subacute cutaneous lupus erythematosus, tumid lupus erythematosus, and verrucous lupus erythematosus (hypertrophic lupus erythematosus)), drug-induced lupus erythematosus, and neonatal lupus. Diseases related to lupus include other systemic autoimmune diseases (e.g., systemic scleroderma, autoimmune myositis, and vasculitis, including Wegener's granulomatosis) or other diseases generally mistaken for lupus (e.g., rheumatoid arthritis, proteinuria, blood disorders, diabetes, fibromyalgia, Lyme disease, and thyroid disease).

The methods, compositions, and diagnostic tests can be used to determine the proper dosage (e.g., the therapeutically effective amount) of a therapeutic agent or to determine the proper type of therapeutic agent to administer to the subject. Any therapeutic agent can be used to treat the subject having, or having a predisposition to, lupus or a related disease (e.g., RA). Exemplary therapeutic agents include acetaminophen, nonsteroidal anti-inflammatory drugs (NSAIDs) (e.g., aspirin, naproxen sodium, or ibuprofen), corticosteroids (e.g., prednisolone), antimalarials (e.g., hydroxychloroquine), and immunosuppressants (e.g., azathioprine, cyclophosphamide, methotrexate, mycophenolate, belimumab, rituximab, epratuzumab, abetimus sodium, abatacept, and BG9588 (an anti-CD40L antibody)).

### Diagnostic Kits

The invention also provides for a diagnostic test kit. For example, a diagnostic test kit can include one or more binding agents (e.g., polynucleotides, such a primers or probes, or polypeptides, such as antibodies), and components for detecting, and more preferably evaluating binding between the binding agent (e.g., a primer, a probe, or an antibody) and the gene or gene product of the invention. In another example, the kit can include a polynucleotide or polypeptide for a gene of the invention, or fragment thereof, for the detection of mRNA or antibodies in the serum or blood of a subject sample that bind to the polynucleotide or polypeptide of the invention. For detection, one or more of the polynucleotide, antibody, or the polypeptide is labeled. In further embodiments, one or more of the polynucleotide, antibody, or the polypeptide is substrate-bound, such that the polypeptide-antibody or polynucleotide-mRNA interaction can be established by determining the amount of label attached to the substrate following binding between the antibody and the polypeptide. A conventional ELISA is a common, art-known method for detecting antibody-substrate interaction and can be provided with the kit of the invention. For detecting the polynucleotide-mRNA interaction, known amplification-based assays can be conducted, such as PCR.

The kit can be used to detect expression level in virtually any bodily fluid, such as urine, plasma, blood serum, semen, or cerebrospinal fluid. A kit that determines an alteration in the level of a polypeptide of the invention relative to a reference, such as the level present in a normal control, is useful as a diagnostic kit in the methods of the invention. Such a kit may further include a reference sample or standard curve indicative of a positive reference or a normal control reference.

Desirably, the kit will contain instructions for the use of the kit. In one example, the kit contains instructions for the use of the kit for the diagnosis of lupus or a propensity to develop the same. In yet another example, the kit contains instructions for the use of the kit to monitor therapeutic treatment or dosage regimens.

In a further example, the instructions include one or more metrics (e.g., principal components) for a principal component analysis that indicates a diagnosis for lupus or a predisposition to develop lupus.

### Screening Assays

As discussed above, we have discovered that the expression level of one or more genes is involved in lupus. Based on these discoveries, one or more of these genes (e.g., IL10) are useful for the high-throughput low-cost screening of candidate compounds to identify those that modulate, alter, or decrease (e.g., by at least 10%, 20%, 30%, 40%, 50%, 60%, 70%, 80%, 90%, 95%, or more), the expression or biological activity of one or more of these genes.

These genes are shown to be up or down regulated by the expression level of the gene or the gene product. Compounds that decrease the expression or biological activity of an activated gene of the invention (e.g., IL10) can be used for the treatment or prevention of lupus or a related disorder (e.g., RA). Compounds that decrease the expression or biological activity of an upregulated gene of the invention (e.g., CD44, CALM3, CD44V3, CD247, HDAC1, CREM, PTGS2, FCER1G, EZR, FOS, IL2, RELA, ICAM1, CD40LG, FASLG, PPP2CB, GATA3, PRKCD, CREB1, IL6, NFATC2, CTLA4, CD40LG, PPP2CB, PRKAR1B, or PRKCQ) can also be used for the treatment or prevention of lupus or a related disorder (e.g., RA).

In general, candidate compounds are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

### Subject Monitoring

The diagnostic methods described herein can also be used to monitor lupus or a related disease (e.g., RA or any described herein) during therapy or to determine the dosage of one or more therapeutic agents. For example, alterations (e.g., an increase or a decrease as compared to the positive reference sample or level for lupus) can be detected to indicate an improvement of the symptoms of lupus. In this embodiment, the levels of the polypeptide, nucleic acid, or antibodies are measured repeatedly as a method of not only diagnosing disease but also monitoring the treatment, prevention, or management of the disease.

In order to monitor the progression of lupus in a subject, subject samples are compared to reference samples taken early in the diagnosis of the disorder. Such monitoring may be useful, for example, in assessing the efficacy of a particular therapeutic agent in a subject, determining dosages, or in assessing disease progression or status. For example, levels of IL10, CD44, CALM3, CD44V3, CD247, HDAC1, CREM, PTGS2, FCER1G, EZR, FOS, IL2, RELA, ICAM1, CD40LG, FASLG, PPP2CB, GATA3, PRKCD, CREB1, IL6, NFATC2, CTLA4, CD40LG, PPP2CB, PRKAR1B, or PRKCQ, or any combination thereof, can be monitored in a patient having lupus and as the levels increase or decrease, relative to control, the dosage or administration of therapeutic agents may be adjusted.

### EXAMPLES

The following examples are intended to illustrate the invention. They are not meant to limit the invention in any way.

### General Procedures

Patients:

Patients (n=10) fulfilling the 4 ACR-established criteria for the diagnosis of SLE were included whereas six patients with an established diagnosis of rheumatoid arthritis (RA) served as disease controls (Table 1). In brief, the age range was 23-56 years old, 90% were women, 30% of Caucasian, 20% African, 20% Hispanic, and 30% of other origin. The age of the RA individuals ranged from 28 to 67 years of age. Nineteen samples from healthy age- and sex and ethnic-matched subjects served as controls. Six patients were studied on two or three occasions during the course of the study. In Table 1, the following symbols are used: A, African American, C, Caucasian, F, female, H, Hispanic, I, Indian, M, male, N, no, Y, yes; *, patients studied on a second or third occasion.

TABLE 1
Demographic, clinical and laboratory features of research subjects.

| | | | Race/ | | | | Anti- | Neuro- | | Musculo- | | | | |
|---|---|---|---|---|---|---|---|---|---|---|---|---|---|---|
| Patient | Age | Sex | Ethnicity | SLEDAI | C3 | C4 | dsDNA | psychiatric | Nephritis | skeletal | Skin | Serositis | Hematologic | Other |
| #1 | 28 | F | C | 12 | | | N | | Y | | | | Y | |
| #2 | 56 | F | C | 0 | 90 | 28 | | | | Y | Y | | | Y |
| #3 | 36 | F | C/A | 0 | 106 | 42 | | | | | Y | | | Y |
| #4 | 30 | F | A/H | 4 | 133 | 28 | Y | | Y | Y | | | | Y |
| #5* | 37 | F | A/A | 10 | 161 | 38 | N | Y | | Y | | Y | | Y |
| | | | | 0 | | | | Y | | Y | | | | |
| #6* | 24 | F | I | 0 | 111 | 18 | N | | Y | Y | Y | | Y | |
| | | | | 0 | 99 | 15 | N | | Y | Y | Y | | Y | |
| | | | | 4 | 91 | 13 | N | | Y | Y | Y | | Y | |
| #7* | 23 | F | A | 35 | 0 | 6 | N | Y | Y | Y | Y | Y | | |
| | | | | 0 | 118 | 35 | N | Y | Y | Y | Y | Y | | |
| #8* | 54 | F | C | 14 | 161 | 37 | N | Y | | Y | Y | | Y | |
| | | | | 0 | | | | Y | | Y | Y | | Y | |
| #9* | 26 | M | A | 2 | 75 | 4 | N | Y | | | Y | | Y | |
| | | | | 4 | 66 | 4 | Y | Y | | | Y | | Y | |
| | | | | 4 | 80 | 4 | Y | Y | | | Y | | Y | |
| #10* | 39 | F | C/H | 0 | 104 | 20 | N | | | | | | | |
| | | | | 10 | 86 | 11 | Y | | Y | Y | | | | Y |
| | | | | 2 | 102 | 18 | Y | | Y | Y | | | | Y |

Basic Design of the SLE Gene Array:

The array was manufactured on a 96-well plate. Each well was embedded with a pair of primers to PCR amplify either 8 housekeeping/control genes (including CD3ε, GAPDH, RTC, HGDC) or a specific gene (n=30) chosen because of claimed importance in the expression of aberrant T cell function in SLE (e.g., see Crispin et al., Trends Mol. Med. 2010; 16(2):47-57 and Kammer et al., Arthritis Rheum. 2002; 46(5):1139-54). Primers for an additional 9 genes claimed to be aberrantly expressed in SLE were embedded but not included in the current analysis. SLE or RA samples were run in parallel to a normal sample on the 96-well plate.

A list of the included genes is shown in Table 2, where the abbreviations stand for the following: IFNA1, Interferon alpha 1; CD247, CD247 molecule; CREM, cAMP responsive element modulator; HDAC1, Histone deacetylase 1; NFATC2, Nuclear factor of activated T cells, cytoplasmic, calcineurin-dependent 2; PTGS2, Prostaglandin-endoperoxide synthase 2 (prostaglandin G/H synthase and cyclooxygenase); IFNA5, Interferon alpha 5; CD3E, CD3e molecule, epsilon (CD3-TCR complex); CTLA4, Cytotoxic T-lymphocyte-associated protein 4; ICAM1, Intercellular adhesion molecule 1 (CD54), human rhinovirus receptor; PDCD1, Programmed cell death 1; ROCK1, Rho-associated, coiled-coil containing protein kinase 1; IL10, Interleukin 10; CD40LG, CD40 ligand (TNF superfamily, member 5, hyper-IgM syndrome); FASLG, Fas ligand (TNF superfamily member 6); IFNG, Interferon gamma; PPP2CA, Protein phosphatase 2 (formerly 2A), catalytic subunit, alpha isoform; SYK, Spleen tyrosine kinase; IL23A, Interleukin 23, alpha subunit p19; CD44, CD44 molecule (Indian blood group); FCER1,G Fc fragment of IgE, high affinity 1, receptor for gamma polypeptide; IL17A, Interleukin 17A; PPP2CB, Protein phosphatase 2 (formerly 2A), catalytic subunit, beta isoform; EZR Ezrin; CD44V3 v3, variant of CD44; FOS, V-fos FBJ murine osteosarcoma viral oncogene homolog; IL17F, Interleukin 17F; PRKAR1B, Protein kinase, cAMP-dependent, regulatory, type I, beta; GAPDH, Glyceraldehyde-3-phosphate dehydrogenase; CD44V6, v6 variant of CD44; FOXP3, Forkhead box P3; IL2, Interleukin 2; PRKAR2B Protein kinase, cAMP-dependent, regulatory, type II, beta; HGDC, Human Genomic DNA Contamination; CD70, CD70 molecule; GATA3, GATA binding protein 3; IL21, Interleukin 21; PRKCD, Protein kinase C, delta; RTC, Reverse Transcription Control; CALM3, Calmodulin 3 (phosphorylase kinase, delta); CREB1, cAMP response element binding protein 1; RELA, V-rel reticuloendotheliosis viral oncogene homolog A, nuclear factor of kappa light polypeptide gene enhancer in B-cells, p65 (avian); IL6, Interleukin 6; PRKCQ, Protein kinase C, theta; and NTC, No template control.

TABLE 2
Layout of the SLE gene expression array

| | 1 | 2 | 3 | 4 | 5 | 6 |
|---|---|---|---|---|---|---|
| A | IFNA1 | CD247 | CREM | HDAC1 | NFATC2 | PTGS2 |
| B | IFNA5 | CD3E | CTLA4 | ICAM1 | PDCD1 | ROCK1 |
| C | IL10 | CD40LG | FASLG | IFNG | PPP2CA | SYK |
| D | IL23A | CD44 | FCER1G | IL17A | PPP2CB | EZR |
| E | FASLG | CD44V3 | FOS | IL17F | PRKAR1B | GAPDH |
| F | GAPDH | CD44V6 | FOXP3 | IL2 | PRKAR2B | HGDC |
| G | HGDC | CD70 | GATA3 | IL21 | PRKCD | RTC |
| H | CALM3 | CREB1 | RELA | IL6 | PRKCQ | NTC |

Determination of Gene Expression Levels:

T cell-derived mRNA (such as described in Krishnan et al., J. Immunol. 2008; 181(11):8145-52 and Katsiari et al., J. Clin. Invest. 2005; 115(11):3193-204) was reversely transcribed to cDNA using the RT2 First Strand Kit (SABiosciences, Frederick, Md.) and placed in the wells of the 96-well plate. Quantitative real time PCR was subsequently performed using the RT² Real-Time SYBR Green PCR Master Mix (SABiosciences, Frederick, Md.) and the product was evaluated utilizing a Roche LightCycler 480 PCR system (Indianapolis, Ind.), which allows gene expression detection within a 10 log interval. Gene expression levels were normalized against the housekeeping gene CD3E. Table 3-5 provides the expression levels for test subjects having lupus and for normal control for each gene. For the top seven genes in Tables 3-5, expression level was measured in total peripheral blood mononuclear cells. For the remaining genes, expression level was measured in T cells. Table 3 shows relative expression levels, Table 4 shows the raw data, and Table 5 shows normalized data (as normalized to CD3E). RTC and HGDC were included as controls, where GAPDH and CD3E were included as housekeeping genes. Fold difference was calculated based on the two-power value of the difference (test-control values). In these tables, higher values correlate with lower expression.

TABLE 3
Expression level for test subjects having
lupus and control (Comparison data)

| | Individuals | Normal | | |
|---|---|---|---|---|
| | with | individuals | Difference | Fold |
| Gene | SLE (average) | (average) | (Test-Control) | difference |
| IFNA1 | 10.6 | 10.5 | 0.1 | 0.96 |
| IFNA5 | 4.9 | −0.6 | 5.5 | 0.02 |
| IL10 | 6.4 | 0.4 | 6.1 | 0.02 |
| IL-23A | 5.2 | 3.8 | 1.4 | 0.38 |
| FASLG | 6.7 | 6.7 | 0.0 | 0.98 |
| GAPDH | −1.3 | 0.0 | −1.3 | 2.51 |
| HGDC | 8.2 | 8.9 | −0.7 | 1.61 |
| CD247 | 1.3 | 1.6 | −0.3 | 1.21 |
| CREM | 3.4 | 4.3 | −0.9 | 1.86 |
| HDAC1 | 1.6 | 1.8 | −0.2 | 1.15 |
| NFATC2 | 5.8 | 6.1 | −0.3 | 1.23 |
| PTGS2 | 2.1 | 3.0 | −0.9 | 1.88 |
| CD3E | −1.1 | 0.0 | −1.1 | 2.08 |
| CTLA4 | 4.0 | 4.5 | −0.5 | 1.37 |
| ICAM1 | 5.9 | 7.2 | −1.2 | 2.35 |
| PDCD1 | 6.4 | 7.2 | −0.8 | 1.70 |
| ROCK1 | 5.3 | 6.0 | −0.7 | 1.62 |
| CD40LG | 2.7 | 3.2 | −0.5 | 1.40 |
| FASLG | 5.7 | 6.7 | −1.0 | 1.96 |
| IFNG | 4.7 | 5.3 | −0.6 | 1.55 |
| PPP2CA | 4.8 | 5.4 | −0.6 | 1.51 |
| SyK | 10.4 | 11.4 | −1.0 | 1.97 |
| CD44 | −0.9 | −0.3 | −0.6 | 1.55 |
| FCER1G | 3.9 | 5.0 | −1.1 | 2.17 |
| IL-17A | 14.8 | — | — | 1.00 |
| PPP2CB | 2.6 | 3.1 | −0.5 | 1.43 |
| EZR | 5.3 | 7.2 | −1.9 | 3.81 |
| CD44V3 | 2.6 | 13.6 | −11.0 | 2076.59 |
| FOS | 9.1 | 11.3 | −2.2 | 4.54 |
| IL-17F | 11.0 | 12.4 | −1.3 | 2.51 |
| PRKAR1B | 6.4 | 6.3 | 0.1 | 0.96 |
| GAPDH | −0.9 | 0.7 | −1.6 | 2.96 |
| CD44V6 | 8.9 | 10.1 | −1.1 | 2.17 |
| FOXP3 | 8.2 | 9.1 | −1.0 | 1.98 |
| IL-2 | 7.7 | 9.4 | −1.7 | 3.17 |
| PRKAR2B | 12.1 | 12.8 | −0.7 | 1.60 |
| HGDC | 7.7 | 9.2 | −1.5 | 2.86 |
| CD70 | 6.9 | 7.9 | −1.0 | 1.99 |
| GATA3 | 7.7 | 9.1 | −1.4 | 2.68 |
| IL-21 | 9.2 | 10.2 | −1.0 | 2.02 |
| PRKCD | 4.1 | 5.0 | −0.8 | 1.80 |
| RTC | −0.5 | 0.2 | −0.7 | 1.64 |
| CALM3 | −0.7 | −0.1 | −0.7 | 1.59 |
| CREB1 | 5.7 | 6.9 | −1.2 | 2.33 |
| RELA | 1.7 | 2.7 | −0.9 | 1.93 |
| IL-6 | 8.1 | 9.8 | −1.7 | 3.25 |
| PRKCQ | 7.4 | 7.2 | 0.2 | 0.86 |

TABLE 4
Expression level for test subjects having lupus and control (Raw data)

| | Normal individuals | | Individuals with SLE | |
|---|---|---|---|---|
| Gene | Ave rage | STD (n = 18) | Average | STD (n = 18) |
| IFNA1 | 33.56 | 1.70 | 33.94 | 1.41 |
| IFNA5 | 33.58 | 1.73 | 34.03 | 2.65 |
| IL10 | 35.11 | 2.00 | 33.59 | 2.09 |
| IL-23A | 30.19 | 1.92 | 30.30 | 2.29 |
| FASLG | 29.71 | 2.48 | 30.07 | 2.62 |
| GAPDH | 23.02 | 2.27 | 22.20 | 2.28 |
| HGDC | 31.90 | 1.10 | 31.57 | 1.10 |
| CD247 | 23.97 | 2.22 | 24.47 | 2.86 |
| CREM | 26.68 | 1.88 | 26.49 | 2.49 |
| HDAC1 | 24.18 | 1.43 | 24.77 | 1.85 |
| NFATC2 | 27.94 | 1.20 | 29.00 | 3.02 |
| PTG2 | 25.38 | 6.05 | 25.82 | 5.79 |
| CD3E | 22.39 | 2.43 | 22.11 | 2.70 |
| CTLA4 | 26.89 | 2.01 | 26.77 | 1.26 |
| ICAM1 | 29.03 | 1.24 | 28.61 | 1.18 |
| PDCD1 | 29.59 | 2.05 | 29.19 | 1.25 |
| ROCK1 | 27.86 | 1.25 | 28.10 | 1.47 |
| CD40LG | 25.56 | 1.82 | 25.42 | 1.48 |
| FASLG | 29.09 | 2.07 | 28.92 | 2.56 |
| IFNG | 27.72 | 1.68 | 27.90 | 2.16 |
| PPP2CA | 27.77 | 1.48 | 27.97 | 1.78 |
| SyK | 33.24 | 2.64 | 33.66 | 2.23 |
| CD44 | 22.14 | 2.09 | 22.31 | 2.29 |
| FCER1G | 27.38 | 1.47 | 27.20 | 1.70 |
| IL-17A | — | — | 35.81 | 0.34 |
| PPP2CB | 25.48 | 1.30 | 25.86 | 1.45 |
| EZR | 29.62 | 2.37 | 28.68 | 2.21 |
| CD44V3 | 35.45 | 1.37 | 36.35 | 1.14 |
| FOS | 33.27 | 1.57 | 32.53 | 2.02 |
| IL-17F | 34.89 | 1.38 | 34.19 | 2.35 |
| PRKAR1B | 28.73 | 2.17 | 29.22 | 1.82 |
| GAPDH | 23.09 | 3.31 | 22.43 | 2.48 |
| CD44V6 | 31.93 | 1.24 | 31.53 | 1.67 |
| FOXP3 | 31.01 | 1.53 | 30.88 | 2.11 |
| IL-2 | 31.75 | 2.01 | 30.86 | 1.35 |
| PRKAR2B | 35.14 | 1.19 | 35.49 | 1.79 |
| HGDC | 31.60 | 1.34 | 30.97 | 1.45 |
| CD70 | 29.72 | 2.22 | 29.63 | 1.48 |
| GATA3 | 31.49 | 1.68 | 30.87 | 2.28 |
| IL-21 | 32.56 | 1.76 | 31.86 | 1.58 |
| PRKCD | 27.36 | 1.22 | 27.38 | 1.14 |
| RTC | 22.65 | 2.61 | 23.11 | 3.15 |
| CALM3 | 22.32 | 2.06 | 22.43 | 2.20 |
| CREB1 | 29.28 | 1.58 | 28.85 | 1.76 |
| RELA | 25.05 | 2.09 | 24.91 | 2.37 |
| IL6 | 31.66 | 2.46 | 31.16 | 2.26 |
| PRKCQ | 29.55 | 1.50 | 30.54 | 2.86 |

TABLE 5
Expression level for test subjects having lupus and control
(Normalized data)

| | Normal individuals | | Individuals with SLE | |
|---|---|---|---|---|
| | Normalized | | Normalized | |
| Gene | Average | STD (n = 18) | Average | (n = 18) |
| IFNA1 | 10.5 | 1.9 | 10.6 | 5.7 |
| IFNA5 | −0.6 | 17.6 | 4.9 | 14.7 |
| IL10 | 0.4 | 18.9 | 6.4 | 13.1 |
| IL-23A | 3.8 | 11.4 | 5.2 | 9.7 |
| FASLG | 6.7 | 1.9 | 6.7 | 5.7 |
| GAPDH | 0.0 | 0.0 | −1.3 | 5.6 |
| HGDC | 8.9 | 2.7 | 8.2 | 6.0 |
| CD247 | 1.6 | 0.5 | 1.3 | 4.6 |
| CREM | 4.3 | 1.4 | 3.4 | 4.4 |
| HDAC1 | 1.8 | 1.5 | 1.6 | 4.7 |
| NFATC2 | 6.1 | 0.6 | 5.8 | 5.1 |
| PTG2 | 3.0 | 6.6 | 2.1 | 8.4 |
| CD3E | 0.0 | 0.0 | −1.1 | 4.5 |
| CTLA4 | 4.5 | 1.0 | 4.0 | 4.7 |
| ICAM1 | 7.2 | 1.0 | 5.9 | 4.7 |
| PDCD1 | 7.2 | 1.2 | 6.4 | 4.8 |
| ROCK1 | 6.0 | 1.1 | 5.3 | 4.9 |
| CD40LG | 3.2 | 0.9 | 2.7 | 4.5 |
| FASLG | 6.7 | 2.0 | 5.7 | 4.6 |
| IFNG | 5.3 | 1.9 | 4.7 | 5.3 |
| PPP2CA | 5.4 | 1.5 | 4.8 | 4.8 |
| SyK | 11.4 | 2.0 | 10.4 | 6.2 |
| CD44 | −0.3 | 0.6 | −0.9 | 4.5 |
| FCER1G | 5.0 | 2.1 | 3.9 | 5.1 |
| IL-17A | — | — | 14.8 | 1.4 |
| PPP2CB | 3.1 | 1.9 | 2.6 | 4.9 |
| EZR | 7.2 | 2.3 | 5.3 | 5.5 |
| CD44V3 | 13.6 | 0.5 | 2.6 | 18.7 |
| FOS | 11.3 | 1.4 | 9.1 | 5.5 |
| IL-17F | 12.4 | 2.3 | 11.0 | 5.3 |
| PRKAR1B | 6.3 | 1.3 | 6.4 | 5.2 |
| GAPDH | 0.7 | 3.1 | −0.9 | 5.2 |
| CD44V6 | 10.1 | 1.1 | 8.9 | 4.6 |
| FOXP3 | 9.1 | 1.2 | 8.2 | 5.2 |
| IL-2 | 9.4 | 1.9 | 7.7 | 4.4 |
| PRKAR2B | 12.8 | 2.2 | 12.1 | 6.2 |
| HGDC | 9.2 | 2.6 | 7.7 | 5.0 |
| CD70 | 7.9 | 1.6 | 6.9 | 5.1 |
| GATA3 | 9.1 | 1.3 | 7.7 | 4.7 |
| IL-21 | 10.2 | 2.8 | 9.2 | 4.7 |
| PRKCD | 5.0 | 1.9 | 4.1 | 4.9 |
| RTC | 0.2 | 2.9 | −0.5 | 5.8 |
| CALM3 | −0.1 | 0.7 | −0.7 | 4.5 |
| CREB1 | 6.9 | 1.9 | 5.7 | 4.7 |
| RELA | 2.7 | 0.9 | 1.7 | 4.5 |
| IL6 | 9.8 | 2.2 | 8.1 | 5.6 |
| PRKCQ | 7.2 | 2.1 | 7.4 | 5.70 |

Statistical Analysis:

Student's t-test was applied to compare the expression of single genes between patients and normal individuals. Principal component analysis (PCA) was applied to identify directions (principal components) along which the variation of the data is maximal, as described in Ringner, Nat. Biotechnol. 2008; 26(3):303-4 and Rencher, Methods of multivariate analysis (2nd ed: Wiley-Interscience; 2002), incorporated herein by reference, using the Matlab (7.0R14, MathWorks) software. In the initial dataset, two individuals displayed exceedingly higher expression values for all genes. To avoid bias, principal components were calculated after excluding these individuals. Representing these individuals on the principal component axes that were calculated in their absence preserved all recorded trends.

### Example 1

### Expression Levels of Genes Detected by the Gene Array

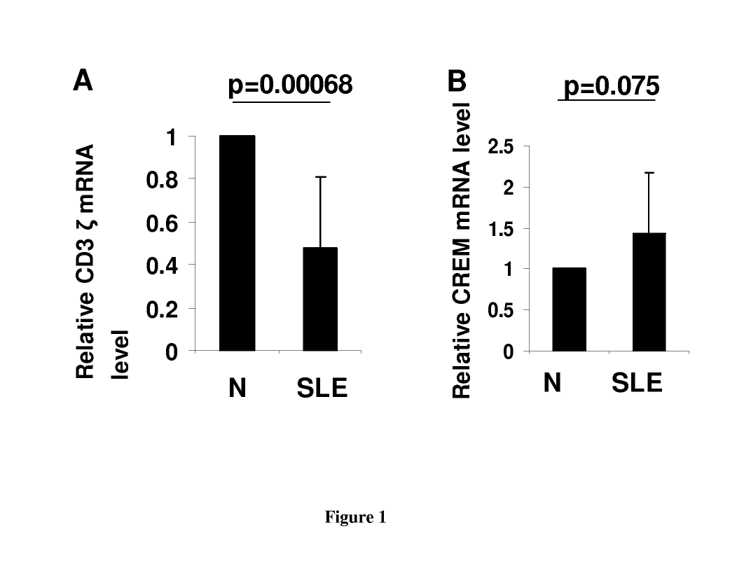

The gene expression array was first designed as a tool to enable the simultaneous determination of the levels of expression of genes to be abnormally expressed and to contribute to the immunopathogenesis of disease. FIGS. 1A-1B show the expression levels of two representative genes, CD3 and CREM, as determined by the SLE gene expression array. As expected, CD3 mRNA levels are decreased and CREM mRNA levels are increased in T cells from patients with SLE, as compared to T cells from sex and age matched normal individuals. The expression levels of all genes in T cells from patients with RA were comparable to those in normal T cells. Accordingly, the SLE gene expression array can be used to detect simultaneously the levels of expression of 30 genes using a small amount of peripheral blood.

### Example 2

### PCA of Expression Levels of Genes Included in the SLE Gene Expression Array

Systemic lupus erythematosus (SLE) presents with fascinating clinical heterogeneity underlined by an equally diverse pathogenic factors and immune system abnormalities. Immune cell abnormalities converge to the production of autoantibodies mostly against nuclear antigens, immune complexes, and T cells which contribute to disease pathology. Disease management still relies on the use of indiscriminate immunosuppression and treatment of arising complications. Progress has been undermined by the absence of tools to classify the disease and measure its activity and proper disease-specific treatment targets.

Aberrant expression of several genes has been implicated in vitro to contribute to the abnormal function of immune cells. For example, correction of the decreased levels of CD3ζ in SLE T cells results in increased production of interleukin 2 (IL-2), inhibition of the increased spleen tyrosine kinase (Syk) levels in SLE T cells results in normal CD3-mediated cell signaling, and inhibition or silencing of increased protein phosphatase 2A (PP2A) results in corrected IL-2 production.

Wishing to capture simultaneously the aberrant expression of all reported genes at a given time point of disease progression using a sensible amount of peripheral blood, we constructed a gene expression array in which we included 30 genes. As described in Example 1, we can capture gene expression variations similar to those reported using classical biochemical approaches. In addition, principal component analysis (PCA) of the expression levels of the included 30 genes placed SLE patients apart from normal subjects and patients with rheumatoid arthritis. Furthermore, distinct clinical manifestations were defined by individual principal components. Accordingly, the gene expression array described herein should facilitate the diagnosis of SLE with improved sensitivity and specificity, and, when larger cohorts of patients have been studied, it may enable a molecular classification of patients that better dictate treatment.

We considered that meaningful phenotypes of the disease would more likely be represented as a function of all genes rather than the separate expression values. To determine whether the included genes contributed to SLE immunopathology, we applied PCA, a mathematical algorithm that organizes data, e.g., gene expression values, into functions (principal components) that better represent the variation between individuals. Each calculated principal component is a function, specifically, a linear combination, of all expression values. For example, if an individual was initially characterized by an expression level e₁ for gene 1 and e₂ for gene 2, a calculated PC would have the form pc₁=c₁e₁+c₂e₂, where c₁ and c₂ are values calculated such that pc₁ describes more of the variation in the sample than either e₁ or e₂ does independently.

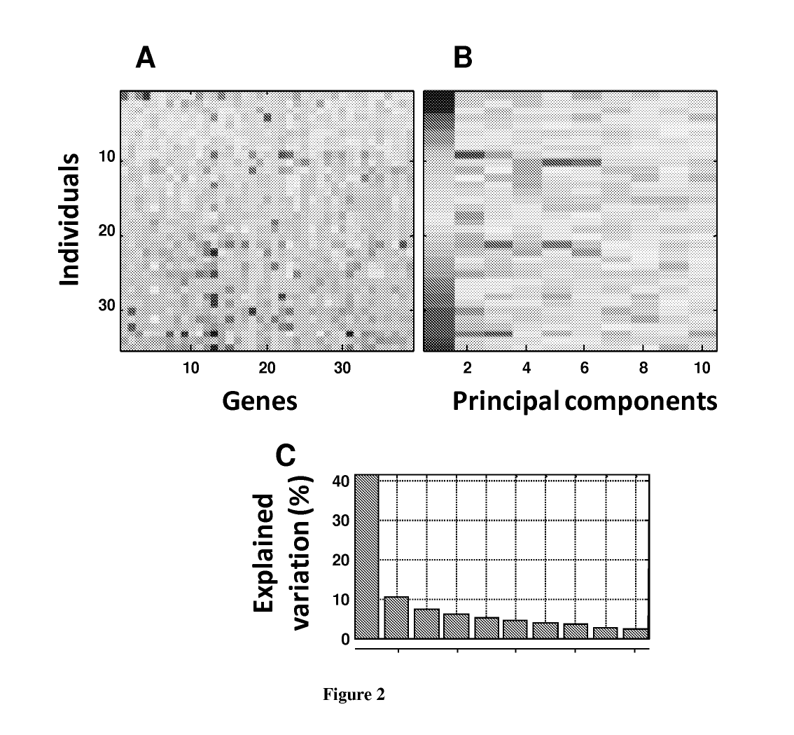

Expression levels for all 30 genes in all studied individuals are shown in FIG. 2A. After applying PCA, principal components were identified and ordered according to their contribution to the overall variance (FIG. 2B). FIG. 2C demonstrates that 42% of the sample variation can be attributed to principal component 1 and as much as 71% of the overall variations can be accounted for by the first 5 principal components and 88% for the first 10 principal components.

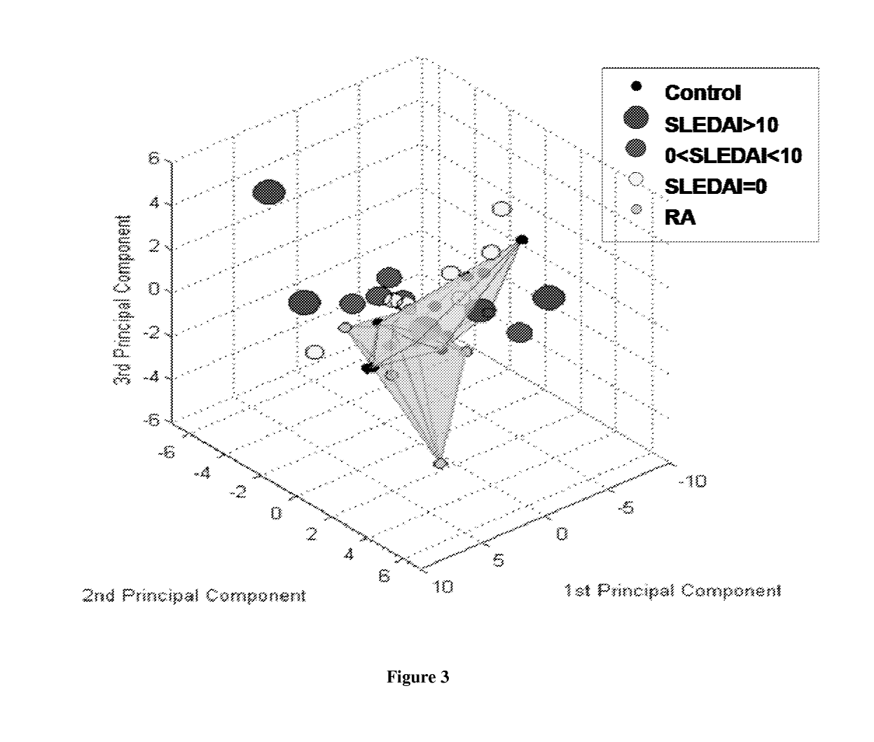

FIG. 3 shows a scatter plot representation of individual samples with the first 3 principal components axes. This plot revealed a striking result whereby the control individuals are spatially separated from the SLE patients. In fact, the variation of control individuals were more constrained and are enclosed by a smaller volume, i.e., a smaller enclosing convex hull. In contrast, SLE patients were far more scattered in these representation axes. Illustrating the clinical and pathogenic complexity of the disease, SLE patient samples were not confined to any specific location and could be roughly classified as having high values in at least one of the principal component axes. Samples from patients with rheumatoid arthritis seemed to localize separately.

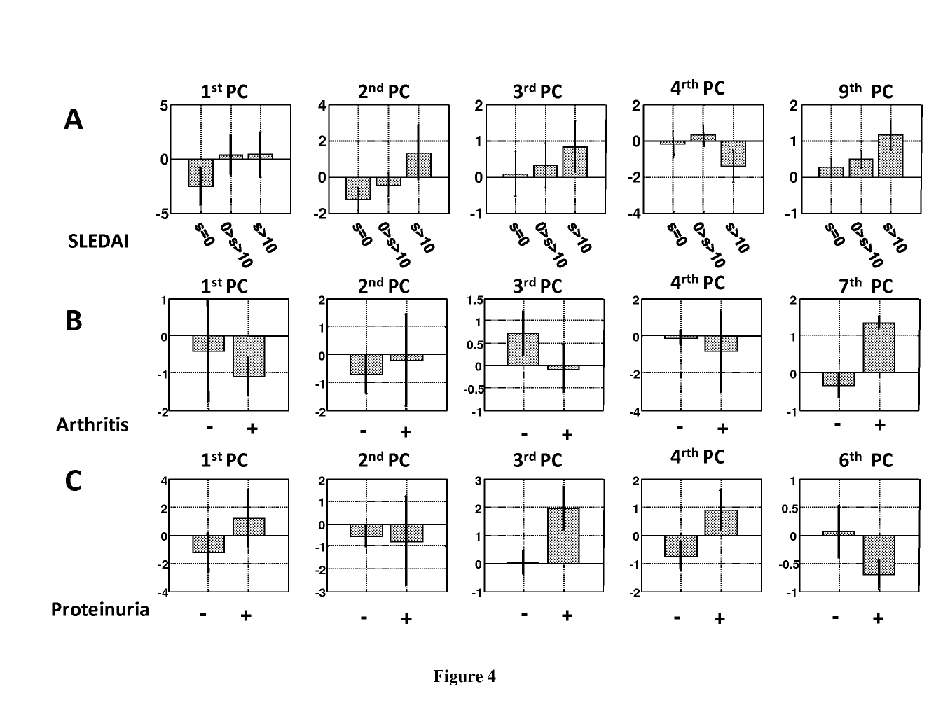

We next asked whether separate individual principal components may represent distinct disease manifestations. We should point out that the calculation of each principal component took place without inputting prior knowledge about the specific diagnosis (controls vs. patients) or clinical manifestation. It was therefore interesting to ask whether any principal component would define a clinically-identified disease feature. It has been frequently demonstrated that principal components may better correlate with clinical features than separate gene expression values. Interestingly, despite our rather small sample size, different principal components appeared to uniquely report different clinical features (FIG. 4). Specifically, FIG. 4A shows that principal components 2 and 9 correlate significantly with SLEDAI scores. In addition, and more interestingly, arthritis is best defined by principal component 7 and proteinuria by principal component 3.

We present here first evidence that a gene expression array consisting of 30 genes that: 1) faithfully reports on the gene expression abnormality in a fashion similar to that reported previously using traditional biochemical approaches, 2) separates in space (using 3 first principal components derived from PCA) the location of SLE samples from those defined by samples from patients with RA and normal individuals, and 3) distinct principal components defined groups of patients with specific clinical manifestations.

While we and others have been studying immune cell biochemistry and molecular biology in patients with SLE in order to identify novel molecular treatment targets and biomarkers, we were challenged physically to record simultaneously the expression of all identified genes at a given time point of the disease. To overcome this difficulty we constructed a gene array, which, even in its first phase, can detect the expression of all genes. For brevity, we report here that the mRNA levels of two genes, CD3ζ and CREM (FIG. 1), were found to be expressed as previously reported.

We considered that the application of PCA would reduce the noise of the heat-map (FIG. 2A) recorded expression levels and identify linear patterns, principal components, which would reduce the number of dimensions of the data to a number that is manageable. Reassuringly, we found that the first principal component contributed by 42% to all variation and the first 5 principal components by 81%. The most surprising finding was that when the first 3 principal components were plotted in a 3-dimensional scattergram, the position of the samples from normal individuals defined a restricted convex hull and only 2 of the 19 SLE samples were located within that space. The samples from RA patients defined a separate space. The 17 lupus samples were positioned outside the space defined by the normal samples regardless of the assigned SLEDAI score suggesting that the 30-gene expression array may very well identify SLE patients who do not have any other clinical manifestations. It remains to be established, among other things, whether the expression array changes position in space as clinical manifestations are added and the ACR-established requirements for the diagnosis of SLE are met.

It is well accepted that an unmet need in field of SLE is the requirement to classify patients in a more accurate manner reflecting better underlying biochemical abnormalities, which may enable properly targeted treatment. When we asked whether any of the calculated principal components define distinct clinical manifestations, we observed that although the SLEDAI score was better represented by principal components 2 and 9, arthritis was defined by principal component 7, and proteinuria by principal component 3. We acknowledge the small number of entries and verification and of our findings with larger numbers of patients is in order, yet, the principal component-defined presence of distinct clinical manifestations is significant (FIG. 4).

Our approach to the identification of gene expression signature is conceptually different from that reported by others, as this array included only genes claimed in in vitro studies to be part of the aberrant SLE T cell function. Overall, this array and other approaches are complementary and can be used to properly diagnose and classify patients with SLE.

Furthermore, SLE samples can be expanded to larger numbers to identify possible effects of treatment and to determine whether principal components can accurately define patients with distinct clinical or laboratory abnormalities. Inclusion of larger numbers representing various ethnic groups can be included in prospective studies, where such studies can be used to determine whether clinical variation in any given patient affects its position in the 3-dimensional space defined by the first 3 or any other combination of principal components.

In conclusion, we present evidence that a gene expression array consisting of genes selected because of their reported importance in the pathogenesis of the disease, can identify SLE patients and define those with distinct clinical manifestations.

SEQUENCE APPENDIX

IL10
>gi|10835141|ref|NP_000563.1| interleukin-10 precursor [Homo sapiens]
(SEQ ID NO: 1)
MHSSALLCCLVLLTGVRASPGQGTQSENSCTHFPGNLPNMLRDLRDAFSRVKTFFQMKDQLDNLLLKESL
LEDFKGYLGCQALSEMIQFYLEEVMPQAENQDPDIKAHVNSLGENLKTLRLRLRRCHRFLPCENKSKAVE
QVKNAFNKLQEKGIYKAMSEFDIFINYIEAYMTMKIRN
>gi|24430216|ref|NM_000572.2| Homo sapiens interleukin 10 (IL10), mRNA
(SEQ ID NO: 2)
ACACATCAGGGGCTTGCTCTTGCAAAACCAAACCACAAGACAGACTTGCAAAAGAAGGCATGCACAGCTC
AGCACTGCTCTGTTGCCTGGTCCTCCTGACTGGGGTGAGGGCCAGCCCAGGCCAGGGCACCCAGTCTGAG
AACAGCTGCACCCACTTCCCAGGCAACCTGCCTAACATGCTTCGAGATCTCCGAGATGCCTTCAGCAGAG
TGAAGACTTTCTTTCAAATGAAGGATCAGCTGGACAACTTGTTGTTAAAGGAGTCCTTGCTGGAGGACTT
TAAGGGTTACCTGGGTTGCCAAGCCTTGTCTGAGATGATCCAGTTTTACCTGGAGGAGGTGATGCCCCAA
GCTGAGAACCAAGACCCAGACATCAAGGCGCATGTGAACTCCCTGGGGGAGAACCTGAAGACCCTCAGGC
TGAGGCTACGGCGCTGTCATCGATTTCTTCCCTGTGAAAACAAGAGCAAGGCCGTGGAGCAGGTGAAGAA
TGCCTTTAATAAGCTCCAAGAGAAAGGCATCTACAAAGCCATGAGTGAGTTTGACATCTTCATCAACTAC
ATAGAAGCCTACATGACAATGAAGATACGAAACTGAGACATCAGGGTGGCGACTCTATAGACTCTAGGAC
ATAAATTAGAGGTCTCCAAAATCGGATCTGGGGCTCTGGGATAGCTGACCCAGCCCCTTGAGAAACCTTA
TTGTACCTCTCTTATAGAATATTTATTACCTCTGATACCTCAACCCCCATTTCTATTTATTTACTGAGCT
TCTCTGTGAACGATTTAGAAAGAAGCCCAATATTATAATTTTTTTCAATATTTATTATTTTCACCTGTTT
TTAAGCTGTTTCCATAGGGTGACACACTATGGTATTTGAGTGTTTTAAGATAAATTATAAGTTACATAAG
GGAGGAAAAAAAATGTTCTTTGGGGAGCCAACAGAAGCTTCCATTCCAAGCCTGACCACGCTTTCTAGCT
GTTGAGCTGTTTTCCCTGACCTCCCTCTAATTTATCTTGTCTCTGGGCTTGGGGCTTCCTAACTGCTACA
AATACTCTTAGGAAGAGAAACCAGGGAGCCCCTTTGATGATTAATTCACCTTCCAGTGTCTCGGAGGGAT
TCCCCTAACCTCATTCCCCAACCACTTCATTCTTGAAAGCTGTGGCCAGCTTGTTATTTATAACAACCTA
AATTTGGTTCTAGGCCGGGCGCGGTGGCTCACGCCTGTAATCCCAGCACTTTGGGAGGCTGAGGCGGGTG
GATCACTTGAGGTCAGGAGTTCCTAACCAGCCTGGTCAACATGGTGAAACCCCGTCTCTACTAAAAATAC
AAAAATTAGCCGGGCATGGTGGCGCGCACCTGTAATCCCAGCTACTTGGGAGGCTGAGGCAAGAGAATTG
CTTGAACCCAGGAGATGGAAGTTGCAGTGAGCTGATATCATGCCCCTGTACTCCAGCCTGGGTGACAGAG
CAAGACTCTGTCTCAAAAAATAAAAATAAAAATAAATTTGGTTCTAATAGAACTCAGTTTTAACTAGAAT
TTATTCAATTCCTCTGGGAATGTTACATTGTTTGTCTGTCTTCATAGCAGATTTTAATTTTGAATAAATA
AATGTATCTTATTCACATC
CD44
>gi|48255935|ref|NP_000601.3| CD44 antigen isoform 1 precursor [Homo
sapiens] (SEQ ID NO: 3)
MDKFWWHAAWGLCLVPLSLAQIDLNITCRFAGVEHVEKNGRYSISRTEAADLCKAFNSTLPTMAQMEKAL
SIGFETCRYGFIEGHVVIPRIHPNSICAANNTGVYILTSNTSQYDTYCFNASAPPEEDCTSVTDLPNAFD
GPITITIVNRDGTRYVQKGEYRTNPEDIYPSNPTDDDVSSGSSSERSSTSGGYIFYTFSTVHPIPDEDSP
WITDSTDRIPATTLMSTSATATETATKRQETWDWFSWLFLPSESKNHLHTTTQMAGTSSNTISAGWEPNE
ENEDERDRHLSFSGSGIDDDEDFISSTISTTPRAFDHTKQNQDWTQWNPSHSNPEVLLQTTTRMTDVDRN
GTTAYEGNWNPEAHPPLIHHEHHEEEETPHSTSTIQATPSSTTEETATQKEQWFGNRWHEGYRQTPKEDS
HSTTGTAAASAHTSHPMQGRTTPSPEDSSWTDFFNPISHPMGRGHQAGRRMDMDSSHSITLQPTANPNTG
LVEDLDRTGPLSMTTQQSNSQSFSTSHEGLEEDKDHPTTSTLTSSNRNDVTGGRRDPNHSEGSTTLLEGY
TSHYPHTKESRTFIPVTSAKTGSFGVTAVTVGDSNSNVNRSLSGDQDTFHPSGGSHTTHGSESDGHSHGS
QEGGANTTSGPIRTPQIPEWLIILASLLALALILAVCIAVNSRRRCGQKKKLVINSGNGAVEDRKPSGLN
GEASKSQEMVHLVNKESSETPDQFMTADETRNLQNVDMKIGV
>gi|48255937|ref|NP_001001389.1| CD44 antigen isoform 2 precursor [Homo
sapiens] (SEQ ID NO: 4)
MDKFWWHAAWGLCLVPLSLAQIDLNITCRFAGVEHVEKNGRYSISRTEAADLCKAFNSTLPTMAQMEKAL
SIGFETCRYGFIEGHVVIPRIHPNSICAANNTGVYILTSNTSQYDTYCFNASAPPEEDCTSVTDLPNAFD
GPITITIVNRDGTRYVQKGEYRTNPEDIYPSNPTDDDVSSGSSSERSSTSGGYIFYTFSTVHPIPDEDSP
WITDSTDRIPATSTSSNTISAGWEPNEENEDERDRHLSFSGSGIDDDEDFISSTISTTPRAFDHTKQNQD
WTQWNPSHSNPEVLLQTTTRMTDVDRNGTTAYEGNWNPEAHPPLIHHEHHEEEETPHSTSTIQATPSSTT
EETATQKEQWFGNRWHEGYRQTPKEDSHSTTGTAAASAHTSHPMQGRTTPSPEDSSWTDFFNPISHPMGR
GHQAGRRMDMDSSHSITLQPTANPNTGLVEDLDRTGPLSMTTQQSNSQSFSTSHEGLEEDKDHPTTSTLT
SSNRNDVTGGRRDPNHSEGSTTLLEGYTSHYPHTKESRTFIPVTSAKTGSFGVTAVTVGDSNSNVNRSLS
GDQDTFHPSGGSHTTHGSESDGHSHGSQEGGANTTSGPIRTPQIPEWLIILASLLALALILAVCIAVNSR
RRCGQKKKLVINSGNGAVEDRKPSGLNGEASKSQEMVHLVNKESSETPDQFMTADETRNLQNVDMKIGV
>gi|48255939|ref|NP_001001390.1| CD44 antigen isoform 3 precursor [Homo
sapiens] (SEQ ID NO: 5)
MDKFWWHAAWGLCLVPLSLAQIDLNITCRFAGVEHVEKNGRYSISRTEAADLCKAFNSTLPTMAQMEKAL
SIGFETCRYGFIEGHVVIPRIHPNSICAANNTGVYILTSNTSQYDTYCFNASAPPEEDCTSVTDLPNAFD
GPITITIVNRDGTRYVQKGEYRTNPEDIYPSNPTDDDVSSGSSSERSSTSGGYIFYTFSTVHPIPDEDSP
WITDSTDRIPATNMDSSHSITLQPTANPNTGLVEDLDRTGPLSMTTQQSNSQSFSTSHEGLEEDKDHPTT
STLTSSNRNDVTGGRRDPNHSEGSTTLLEGYTSHYPHTKESRTFIPVTSAKTGSFGVTAVTVGDSNSNVN
RSLSGDQDTFHPSGGSHTTHGSESDGHSHGSQEGGANTTSGPIRTPQIPEWLIILASLLALALILAVCIA
VNSRRRCGQKKKLVINSGNGAVEDRKPSGLNGEASKSQEMVHLVNKESSETPDQFMTADETRNLQNVDMK
IGV
>gi|48255941|ref|NP_001001391.1| CD44 antigen isoform 4 precursor [Homo
sapiens] (SEQ ID NO: 6)
MDKFWWHAAWGLCLVPLSLAQIDLNITCRFAGVFHVEKNGRYSISRTEAADLCKAFNSTLPTMAQMEKAL
SIGFETCRYGFIEGHVVIPRIHPNSICAANNTGVYILTSNTSQYDTYCFNASAPPEEDCTSVTDLPNAFD
GPITITIVNRDGTRYVQKGEYRTNPEDIYPSNPTDDDVSSGSSSERSSTSGGYIFYTFSTVHPIPDEDSP
WITDSTDRIPATRDQDTFHPSGGSHTTHGSESDGHSHGSQEGGANTTSGPIRTPQIPEWLIILASLLALA
LILAVCIAVNSRRRCGQKKKLVINSGNGAVEDRKPSGLNGEASKSQEMVHLVNKESSETPDQFMTADETR
NLQNVDMKIGV
>gi|48255943|ref|NP_001001392.1| CD44 antigen isoform 5 precursor [Homo
sapiens] (SEQ ID NO: 7)
MDKFWWHAAWGLCLVPLSLAQIDLNITCRFAGVFHVEKNGRYSISRTEAADLCKAFNSTLPTMAQMEKAL
SIGFETCSLHCSQQSKKVWAEEKASDQQWQWSCGGQKAKWTQRRGQQVSGNGAFGEQGVVRNSRPVYDS
>gi|321400138|ref|NP_001189484.1| CD44 antigen isoform 6 precursor [Homo
sapiens] (SEQ ID NO: 8)
MDKFWWHAAWGLCLVPLSLAQIDLNITCRFAGVFHVEKNGRYSISRTEAADLCKAFNSTLPTMAQMEKAL
SIGFETCRYGFIEGHVVIPRIHPNSICAANNTGVYILTSNTSQYDTYCFNASAPPEEDCTSVTDLPNAFD
GPITITIVNRDGTRYVQKGEYRTNPEDIYPSNPTDDDVSSGSSSERSSTSGGYIFYTFSTVHPIPDEDSP
WITDSTDRIPATNRNDVTGGRRDPNHSEGSTTLLEGYTSHYPHTKESRTFIPVTSAKTGSFGVTAVTVGD
SNSNVNRSLSGDQDTFHPSGGSHTTHGSESDGHSHGSQEGGANTTSGPIRTPQIPEWLIILASLLALALI
LAVCIAVNSRRRCGQKKKLVINSGNGAVEDRKPSGLNGEASKSQEMVHLVNKESSETPDQFMTADETRNL
QNVDMKIGV
>gi|321400140|ref|NP_001189485.1| CD44 antigen isoform 7 precursor [Homo
sapiens] (SEQ ID NO: 9)
MDKFWWHAAWGLCLVPLSLAQIDLNITCRFAGVFHVEKNGRYSISRTEAADLCKAFNSTLPTMAQMEKAL
SIGFETCRYGFIEGHVVIPRIHPNSICAANNTGVYILTSNTSQYDTYCFNASAPPEEDCTSVTDLPNAFD
GPITITIVNRDGTRYVQKGEYRTNPEDIYPSNPTDDDVSSGSSSERSSTSGGYIFYTFSTVHPIPDEDSP
WITDSTDRIPATRHSHGSQEGGANTTSGPIRTPQIPEWLIILASLLALALILAVCIAVNSRRRCGQKKKL
VINSGNGAVEDRKPSGLNGEASKSQEMVHLVNKESSETPDQFMTADETRNLQNVDMKIGV
>gi|321400142|ref|NP_001189486.1| CD44 antigen isoform 8 precursor [Homo
sapiens] (SEQ ID NO: 10)
MDKFWWHAAWGLCLVPLSLAQIDLNITCRFAGVFHVEKNGRYSISRTEAADLCKAFNSTLPTMAQMEKAL
SIGFETCRYGFIEGHVVIPRIHPNSICAANNTGVYILTSNTSQYDTYCFNASAPPEEDCTSVTDLPNAFD
GPITITIVNRDGTRYVQKGEYRTNPEDIYPSNPTDDDVSSGSSSERSSTSGGYIFYTFSTVHPIPDEDSP
WITDSTDRIPATRDQDTFHPSGGSHTTHGSESDGHSHGSQEGGANTTSGPIRTPQIPEWLIILASLLALA
LILAVCIAVNSRRS
>gi|48255934|ref|NM_000610.3| Homo sapiens CD44 molecule (Indian blood
group) (CD44), transcript variant 1, mRNA (SEQ ID NO: 11)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGGTATGGGTTCATAGAAGGGCACGTGGTGATTCC
CCGGATCCACCCCAACTCCATCTGTGCAGCAAACAACACAGGGGTGTACATCCTCACATCCAACACCTCC
CAGTATGACACATATTGCTTCAATGCTTCAGCTCCACCTGAAGAAGATTGTACATCAGTCACAGACCTGC
CCAATGCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCGGCTCC
TCCAGTGAAAGGAGCAGCACTTCAGGAGGTTACATCTTTTACACCTTTTCTACTGTACACCCCATCCCAG
ACGAAGACAGTCCCTGGATCACCGACAGCACAGACAGAATCCCTGCTACCACTTTGATGAGCACTAGTGC
TACAGCAACTGAGACAGCAACCAAGAGGCAAGAAACCTGGGATTGGTTTTCATGGTTGTTTCTACCATCA
GAGTCAAAGAATCATCTTCACACAACAACACAAATGGCTGGTACGTCTTCAAATACCATCTCAGCAGGCT
GGGAGCCAAATGAAGAAAATGAAGATGAAAGAGACAGACACCTCAGTTTTTCTGGATCAGGCATTGATGA
TGATGAAGATTTTATCTCCAGCACCATTTCAACCACACCACGGGCTTTTGACCACACAAAACAGAACCAG
GACTGGACCCAGTGGAACCCAAGCCATTCAAATCCGGAAGTGCTACTTCAGACAACCACAAGGATGACTG
ATGTAGACAGAAATGGCACCACTGCTTATGAAGGAAACTGGAACCCAGAAGCACACCCTCCCCTCATTCA
CCATGAGCATCATGAGGAAGAAGAGACCCCACATTCTACAAGCACAATCCAGGCAACTCCTAGTAGTACA
ACGGAAGAAACAGCTACCCAGAAGGAACAGTGGTTTGGCAACAGATGGCATGAGGGATATCGCCAAACAC
CCAAAGAAGACTCCCATTCGACAACAGGGACAGCTGCAGCCTCAGCTCATACCAGCCATCCAATGCAAGG
AAGGACAACACCAAGCCCAGAGGACAGTTCCTGGACTGATTTCTTCAACCCAATCTCACACCCCATGGGA
CGAGGTCATCAAGCAGGAAGAAGGATGGATATGGACTCCAGTCATAGTATAACGCTTCAGCCTACTGCAA
ATCCAAACACAGGTTTGGTGGAAGATTTGGACAGGACAGGACCTCTTTCAATGACAACGCAGCAGAGTAA
TTCTCAGAGCTTCTCTACATCACATGAAGGCTTGGAAGAAGATAAAGACCATCCAACAACTTCTACTCTG
ACATCAAGCAATAGGAATGATGTCACAGGTGGAAGAAGAGACCCAAATCATTCTGAAGGCTCAACTACTT
TACTGGAAGGTTATACCTCTCATTACCCACACACGAAGGAAAGCAGGACCTTCATCCCAGTGACCTCAGC
TAAGACTGGGTCCTTTGGAGTTACTGCAGTTACTGTTGGAGATTCCAACTCTAATGTCAATCGTTCCTTA
TCAGGAGACCAAGACACATTCCACCCCAGTGGGGGGTCCCATACCACTCATGGATCTGAATCAGATGGAC
ACTCACATGGGAGTCAAGAAGGTGGAGCAAACACAACCTCTGGTCCTATAAGGACACCCCAAATTCCAGA
ATGGCTGATCATCTTGGCATCCCTCTTGGCCTTGGCTTTGATTCTTGCAGTTTGCATTGCAGTCAACAGT
CGAAGAAGGTGTGGGCAGAAGAAAAAGCTAGTGATCAACAGTGGCAATGGAGCTGTGGAGGACAGAAAGC
CAAGTGGACTCAACGGAGAGGCCAGCAAGTCTCAGGAAATGGTGCATTTGGTGAACAAGGAGTCGTCAGA
AACTCCAGACCAGTTTATGACAGCTGATGAGACAAGGAACCTGCAGAATGTGGACATGAAGATTGGGGTG
TAACACCTACACCATTATCTTGGAAAGAAACAACCGTTGGAAACATAACCATTACAGGGAGCTGGGACAC
TTAACAGATGCAATGTGCTACTGATTGTTTCATTGCGAATCTTTTTTAGCATAAAATTTTCTACTCTTTT
TGTTTTTTGTGTTTTGTTCTTTAAAGTCAGGTCCAATTTGTAAAAACAGCATTGCTTTCTGAAATTAGGG
CCCAATTAATAATCAGCAAGAATTTGATCGTTCCAGTTCCCACTTGGAGGCCTTTCATCCCTCGGGTGTG
CTATGGATGGCTTCTAACAAAAACTACACATATGTATTCCTGATCGCCAACCTTTCCCCCACCAGCTAAG
GACATTTCCCAGGGTTAATAGGGCCTGGTCCCTGGGAGGAAATTTGAATGGGTCCATTTTGCCCTTCCAT
AGCCTAATCCCTGGGCATTGCTTTCCACTGAGGTTGGGGGTTGGGGTGTACTAGTTACACATCTTCAACA
GACCCCCTCTAGAAATTTTTCAGATGCTTCTGGGAGACACCCAAAGGGTGAAGCTATTTATCTGTAGTAA
ACTATTTATCTGTGTTTTTGAAATATTAAACCCTGGATCAGTCCTTTGATCAGTATAATTTTTTAAAGTT
ACTTTGTCAGAGGCACAAAAGGGTTTAAACTGATTCATAATAAATATCTGTACTTCTTCGATCTTCACCT
TTTGTGCTGTGATTCTTCAGTTTCTAAACCAGCACTGTCTGGGTCCCTACAATGTATCAGGAAGAGCTGA
GAATGGTAAGGAGACTCTTCTAAGTCTTCATCTCAGAGACCCTGAGTTCCCACTCAGACCCACTCAGCCA
AATCTCATGGAAGACCAAGGAGGGCAGCACTGTTTTTGTTTTTTGTTTTTTGTTTTTTTTTTTTGACACT
GTCCAAAGGTTTTCCATCCTGTCCTGGAATCAGAGTTGGAAGCTGAGGAGCTTCAGCCTCTTTTATGGTT
TAATGGCCACCTGTTCTCTCCTGTGAAAGGCTTTGCAAAGTCACATTAAGTTTGCATGACCTGTTATCCC
TGGGGCCCTATTTCATAGAGGCTGGCCCTATTAGTGATTTCCAAAAACAATATGGAAGTGCCTTTTGATG
TCTTACAATAAGAGAAGAAGCCAATGGAAATGAAAGAGATTGGCAAAGGGGAAGGATGATGCCATGTAGA
TCCTGTTTGACATTTTTATGGCTGTATTTGTAAACTTAAACACACCAGTGTCTGTTCTTGATGCAGTTGC
TATTTAGGATGAGTTAAGTGCCTGGGGAGTCCCTCAAAAGGTTAAAGGGATTCCCATCATTGGAATCTTA
TCACCAGATAGGCAAGTTTATGACCAAACAAGAGAGTACTGGCTTTATCCTCTAACCTCATATTTTCTCC
CACTTGGCAAGTCCTTTGTGGCATTTATTCATCAGTCAGGGTGTCCGATTGGTCCTAGAACTTCCAAAGG
CTGCTTGTCATAGAAGCCATTGCATCTATAAAGCAACGGCTCCTGTTAAATGGTATCTCCTTTCTGAGGC
TCCTACTAAAAGTCATTTGTTACCTAAACTTATGTGCTTAACAGGCAATGCTTCTCAGACCACAAAGCAG
AAAGAAGAAGAAAAGCTCCTGACTAAATCAGGGCTGGGCTTAGACAGAGTTGATCTGTAGAATATCTTTA
AAGGAGAGATGTCAACTTTCTGCACTATTCCCAGCCTCTGCTCCTCCCTGTCTACCCTCTCCCCTCCCTC
TCTCCCTCCACTTCACCCCACAATCTTGAAAAACTTCCTTTCTCTTCTGTGAACATCATTGGCCAGATCC
ATTTTCAGTGGTCTGGATTTCTTTTTATTTTCTTTTCAACTTGAAAGAAACTGGACATTAGGCCACTATG
TGTTGTTACTGCCACTAGTGTTCAAGTGCCTCTTGTTTTCCCAGAGATTTCCTGGGTCTGCCAGAGGCCC
AGACAGGCTCACTCAAGCTCTTTAACTGAAAAGCAACAAGCCACTCCAGGACAAGGTTCAAAATGGTTAC
AACAGCCTCTACCTGTCGCCCCAGGGAGAAAGGGGTAGTGATACAAGTCTCATAGCCAGAGATGGTTTTC
CACTCCTTCTAGATATTCCCAAAAAGAGGCTGAGACAGGAGGTTATTTTCAATTTTATTTTGGAATTAAA
TACTTTTTTCCCTTTATTACTGTTGTAGTCCCTCACTTGGATATACCTCTGTTTTCACGATAGAAATAAG
GGAGGTCTAGAGCTTCTATTCCTTGGCCATTGTCAACGGAGAGCTGGCCAAGTCTTCACAAACCCTTGCA
ACATTGCCTGAAGTTTATGGAATAAGATGTATTCTCACTCCCTTGATCTCAAGGGCGTAACTCTGGAAGC
ACAGCTTGACTACACGTCATTTTTACCAATGATTTTCAGGTGACCTGGGCTAAGTCATTTAAACTGGGTC
TTTATAAAAGTAAAAGGCCAACATTTAATTATTTTGCAAAGCAACCTAAGAGCTAAAGATGTAATTTTTC
TTGCAATTGTAAATCTTTTGTGTCTCCTGAAGACTTCCCTTAAAATTAGCTCTGAGTGAAAAATCAAAAG
AGACAAAAGACATCTTCGAATCCATATTTCAAGCCTGGTAGAATTGGCTTTTCTAGCAGAACCTTTCCAA
AAGTTTTATATTGAGATTCATAACAACACCAAGAATTGATTTTGTAGCCAACATTCATTCAATACTGTTA
TATCAGAGGAGTAGGAGAGAGGAAACATTTGACTTATCTGGAAAAGCAAAATGTACTTAAGAATAAGAAT
AACATGGTCCATTCACCTTTATGTTATAGATATGTCTTTGTGTAAATCATTTGTTTTGAGTTTTCAAAGA
ATAGCCCATTGTTCATTCTTGTGCTGTACAATGACCACTGTTATTGTTACTTTGACTTTTCAGAGCACAC
CCTTCCTCTGGTTTTTGTATATTTATTGATGGATCAATAATAATGAGGAAAGCATGATATGTATATTGCT
GAGTTGAAAGCACTTATTGGAAAATATTAAAAGGCTAACATTAAAAGACTAAAGGAAACAGAAAAAAAAA
AAAAAAAA
>gi|48255936|ref|NM_001001389.1| Homo sapiens CD44 molecule (Indian
blood group) (CD44), transcript variant 2, mRNA (SEQ ID NO: 12)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGGTATGGGTTCATAGAAGGGCACGTGGTGATTCC
CCGGATCCACCCCAACTCCATCTGTGCAGCAAACAACACAGGGGTGTACATCCTCACATCCAACACCTCC
CAGTATGACACATATTGCTTCAATGCTTCAGCTCCACCTGAAGAAGATTGTACATCAGTCACAGACCTGC
CCAATGCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCGGCTCC
TCCAGTGAAAGGAGCAGCACTTCAGGAGGTTACATCTTTTACACCTTTTCTACTGTACACCCCATCCCAG
ACGAAGACAGTCCCTGGATCACCGACAGCACAGACAGAATCCCTGCTACCAGTACGTCTTCAAATACCAT
CTCAGCAGGCTGGGAGCCAAATGAAGAAAATGAAGATGAAAGAGACAGACACCTCAGTTTTTCTGGATCA
GGCATTGATGATGATGAAGATTTTATCTCCAGCACCATTTCAACCACACCACGGGCTTTTGACCACACAA
AACAGAACCAGGACTGGACCCAGTGGAACCCAAGCCATTCAAATCCGGAAGTGCTACTTCAGACAACCAC
AAGGATGACTGATGTAGACAGAAATGGCACCACTGCTTATGAAGGAAACTGGAACCCAGAAGCACACCCT
CCCCTCATTCACCATGAGCATCATGAGGAAGAAGAGACCCCACATTCTACAAGCACAATCCAGGCAACTC
CTAGTAGTACAACGGAAGAAACAGCTACCCAGAAGGAACAGTGGTTTGGCAACAGATGGCATGAGGGATA
TCGCCAAACACCCAAAGAAGACTCCCATTCGACAACAGGGACAGCTGCAGCCTCAGCTCATACCAGCCAT
CCAATGCAAGGAAGGACAACACCAAGCCCAGAGGACAGTTCCTGGACTGATTTCTTCAACCCAATCTCAC
ACCCCATGGGACGAGGTCATCAAGCAGGAAGAAGGATGGATATGGACTCCAGTCATAGTATAACGCTTCA
GCCTACTGCAAATCCAAACACAGGTTTGGTGGAAGATTTGGACAGGACAGGACCTCTTTCAATGACAACG
CAGCAGAGTAATTCTCAGAGCTTCTCTACATCACATGAAGGCTTGGAAGAAGATAAAGACCATCCAACAA
CTTCTACTCTGACATCAAGCAATAGGAATGATGTCACAGGTGGAAGAAGAGACCCAAATCATTCTGAAGG
CTCAACTACTTTACTGGAAGGTTATACCTCTCATTACCCACACACGAAGGAAAGCAGGACCTTCATCCCA
GTGACCTCAGCTAAGACTGGGTCCTTTGGAGTTACTGCAGTTACTGTTGGAGATTCCAACTCTAATGTCA
ATCGTTCCTTATCAGGAGACCAAGACACATTCCACCCCAGTGGGGGGTCCCATACCACTCATGGATCTGA
ATCAGATGGACACTCACATGGGAGTCAAGAAGGTGGAGCAAACACAACCTCTGGTCCTATAAGGACACCC
CAAATTCCAGAATGGCTGATCATCTTGGCATCCCTCTTGGCCTTGGCTTTGATTCTTGCAGTTTGCATTG
CAGTCAACAGTCGAAGAAGGTGTGGGCAGAAGAAAAAGCTAGTGATCAACAGTGGCAATGGAGCTGTGGA
GGACAGAAAGCCAAGTGGACTCAACGGAGAGGCCAGCAAGTCTCAGGAAATGGTGCATTTGGTGAACAAG
GAGTCGTCAGAAACTCCAGACCAGTTTATGACAGCTGATGAGACAAGGAACCTGCAGAATGTGGACATGA
AGATTGGGGTGTAACACCTACACCATTATCTTGGAAAGAAACAACCGTTGGAAACATAACCATTACAGGG
AGCTGGGACACTTAACAGATGCAATGTGCTACTGATTGTTTCATTGCGAATCTTTTTTAGCATAAAATTT
TCTACTCTTTTTGTTTTTTGTGTTTTGTTCTTTAAAGTCAGGTCCAATTTGTAAAAACAGCATTGCTTTC
TGAAATTAGGGCCCAATTAATAATCAGCAAGAATTTGATCGTTCCAGTTCCCACTTGGAGGCCTTTCATC
CCTCGGGTGTGCTATGGATGGCTTCTAACAAAAACTACACATATGTATTCCTGATCGCCAACCTTTCCCC
CACCAGCTAAGGACATTTCCCAGGGTTAATAGGGCCTGGTCCCTGGGAGGAAATTTGAATGGGTCCATTT
TGCCCTTCCATAGCCTAATCCCTGGGCATTGCTTTCCACTGAGGTTGGGGGTTGGGGTGTACTAGTTACA
CATCTTCAACAGACCCCCTCTAGAAATTTTTCAGATGCTTCTGGGAGACACCCAAAGGGTGAAGCTATTT
ATCTGTAGTAAACTATTTATCTGTGTTTTTGAAATATTAAACCCTGGATCAGTCCTTTGATCAGTATAAT
TTTTTAAAGTTACTTTGTCAGAGGCACAAAAGGGTTTAAACTGATTCATAATAAATATCTGTACTTCTTC
GATCTTCACCTTTTGTGCTGTGATTCTTCAGTTTCTAAACCAGCACTGTCTGGGTCCCTACAATGTATCA
GGAAGAGCTGAGAATGGTAAGGAGACTCTTCTAAGTCTTCATCTCAGAGACCCTGAGTTCCCACTCAGAC
CCACTCAGCCAAATCTCATGGAAGACCAAGGAGGGCAGCACTGTTTTTGTTTTTTGTTTTTTGTTTTTTT
TTTTTGACACTGTCCAAAGGTTTTCCATCCTGTCCTGGAATCAGAGTTGGAAGCTGAGGAGCTTCAGCCT
CTTTTATGGTTTAATGGCCACCTGTTCTCTCCTGTGAAAGGCTTTGCAAAGTCACATTAAGTTTGCATGA
CCTGTTATCCCTGGGGCCCTATTTCATAGAGGCTGGCCCTATTAGTGATTTCCAAAAACAATATGGAAGT
GCCTTTTGATGTCTTACAATAAGAGAAGAAGCCAATGGAAATGAAAGAGATTGGCAAAGGGGAAGGATGA
TGCCATGTAGATCCTGTTTGACATTTTTATGGCTGTATTTGTAAACTTAAACACACCAGTGTCTGTTCTT
GATGCAGTTGCTATTTAGGATGAGTTAAGTGCCTGGGGAGTCCCTCAAAAGGTTAAAGGGATTCCCATCA
TTGGAATCTTATCACCAGATAGGCAAGTTTATGACCAAACAAGAGAGTACTGGCTTTATCCTCTAACCTC
ATATTTTCTCCCACTTGGCAAGTCCTTTGTGGCATTTATTCATCAGTCAGGGTGTCCGATTGGTCCTAGA
ACTTCCAAAGGCTGCTTGTCATAGAAGCCATTGCATCTATAAAGCAACGGCTCCTGTTAAATGGTATCTC
CTTTCTGAGGCTCCTACTAAAAGTCATTTGTTACCTAAACTTATGTGCTTAACAGGCAATGCTTCTCAGA
CCACAAAGCAGAAAGAAGAAGAAAAGCTCCTGACTAAATCAGGGCTGGGCTTAGACAGAGTTGATCTGTA
GAATATCTTTAAAGGAGAGATGTCAACTTTCTGCACTATTCCCAGCCTCTGCTCCTCCCTGTCTACCCTC
TCCCCTCCCTCTCTCCCTCCACTTCACCCCACAATCTTGAAAAACTTCCTTTCTCTTCTGTGAACATCAT
TGGCCAGATCCATTTTCAGTGGTCTGGATTTCTTTTTATTTTCTTTTCAACTTGAAAGAAACTGGACATT
AGGCCACTATGTGTTGTTACTGCCACTAGTGTTCAAGTGCCTCTTGTTTTCCCAGAGATTTCCTGGGTCT
GCCAGAGGCCCAGACAGGCTCACTCAAGCTCTTTAACTGAAAAGCAACAAGCCACTCCAGGACAAGGTTC
AAAATGGTTACAACAGCCTCTACCTGTCGCCCCAGGGAGAAAGGGGTAGTGATACAAGTCTCATAGCCAG
AGATGGTTTTCCACTCCTTCTAGATATTCCCAAAAAGAGGCTGAGACAGGAGGTTATTTTCAATTTTATT
TTGGAATTAAATACTTTTTTCCCTTTATTACTGTTGTAGTCCCTCACTTGGATATACCTCTGTTTTCACG
ATAGAAATAAGGGAGGTCTAGAGCTTCTATTCCTTGGCCATTGTCAACGGAGAGCTGGCCAAGTCTTCAC
AAACCCTTGCAACATTGCCTGAAGTTTATGGAATAAGATGTATTCTCACTCCCTTGATCTCAAGGGCGTA
ACTCTGGAAGCACAGCTTGACTACACGTCATTTTTACCAATGATTTTCAGGTGACCTGGGCTAAGTCATT
TAAACTGGGTCTTTATAAAAGTAAAAGGCCAACATTTAATTATTTTGCAAAGCAACCTAAGAGCTAAAGA
TGTAATTTTTCTTGCAATTGTAAATCTTTTGTGTCTCCTGAAGACTTCCCTTAAAATTAGCTCTGAGTGA
AAAATCAAAAGAGACAAAAGACATCTTCGAATCCATATTTCAAGCCTGGTAGAATTGGCTTTTCTAGCAG
AACCTTTCCAAAAGTTTTATATTGAGATTCATAACAACACCAAGAATTGATTTTGTAGCCAACATTCATT
CAATACTGTTATATCAGAGGAGTAGGAGAGAGGAAACATTTGACTTATCTGGAAAAGCAAAATGTACTTA
AGAATAAGAATAACATGGTCCATTCACCTTTATGTTATAGATATGTCTTTGTGTAAATCATTTGTTTTGA
GTTTTCAAAGAATAGCCCATTGTTCATTCTTGTGCTGTACAATGACCACTGTTATTGTTACTTTGACTTT
TCAGAGCACACCCTTCCTCTGGTTTTTGTATATTTATTGATGGATCAATAATAATGAGGAAAGCATGATA
TGTATATTGCTGAGTTGAAAGCACTTATTGGAAAATATTAAAAGGCTAACATTAAAAGACTAAAGGAAAC
AGAAAAAAAAAAAAAAAAA
>gi|48255938|ref|NM_001001390.1| Homo sapiens CD44 molecule (Indian
blood group) (CD44), transcript variant 3, mRNA (SEQ ID NO: 13)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGGTATGGGTTCATAGAAGGGCACGTGGTGATTCC
CCGGATCCACCCCAACTCCATCTGTGCAGCAAACAACACAGGGGTGTACATCCTCACATCCAACACCTCC
CAGTATGACACATATTGCTTCAATGCTTCAGCTCCACCTGAAGAAGATTGTACATCAGTCACAGACCTGC
CCAATGCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCGGCTCC
TCCAGTGAAAGGAGCAGCACTTCAGGAGGTTACATCTTTTACACCTTTTCTACTGTACACCCCATCCCAG
ACGAAGACAGTCCCTGGATCACCGACAGCACAGACAGAATCCCTGCTACCAATATGGACTCCAGTCATAG
TATAACGCTTCAGCCTACTGCAAATCCAAACACAGGTTTGGTGGAAGATTTGGACAGGACAGGACCTCTT
TCAATGACAACGCAGCAGAGTAATTCTCAGAGCTTCTCTACATCACATGAAGGCTTGGAAGAAGATAAAG
ACCATCCAACAACTTCTACTCTGACATCAAGCAATAGGAATGATGTCACAGGTGGAAGAAGAGACCCAAA
TCATTCTGAAGGCTCAACTACTTTACTGGAAGGTTATACCTCTCATTACCCACACACGAAGGAAAGCAGG
ACCTTCATCCCAGTGACCTCAGCTAAGACTGGGTCCTTTGGAGTTACTGCAGTTACTGTTGGAGATTCCA
ACTCTAATGTCAATCGTTCCTTATCAGGAGACCAAGACACATTCCACCCCAGTGGGGGGTCCCATACCAC
TCATGGATCTGAATCAGATGGACACTCACATGGGAGTCAAGAAGGTGGAGCAAACACAACCTCTGGTCCT
ATAAGGACACCCCAAATTCCAGAATGGCTGATCATCTTGGCATCCCTCTTGGCCTTGGCTTTGATTCTTG
CAGTTTGCATTGCAGTCAACAGTCGAAGAAGGTGTGGGCAGAAGAAAAAGCTAGTGATCAACAGTGGCAA
TGGAGCTGTGGAGGACAGAAAGCCAAGTGGACTCAACGGAGAGGCCAGCAAGTCTCAGGAAATGGTGCAT
TTGGTGAACAAGGAGTCGTCAGAAACTCCAGACCAGTTTATGACAGCTGATGAGACAAGGAACCTGCAGA
ATGTGGACATGAAGATTGGGGTGTAACACCTACACCATTATCTTGGAAAGAAACAACCGTTGGAAACATA
ACCATTACAGGGAGCTGGGACACTTAACAGATGCAATGTGCTACTGATTGTTTCATTGCGAATCTTTTTT
AGCATAAAATTTTCTACTCTTTTTGTTTTTTGTGTTTTGTTCTTTAAAGTCAGGTCCAATTTGTAAAAAC
AGCATTGCTTTCTGAAATTAGGGCCCAATTAATAATCAGCAAGAATTTGATCGTTCCAGTTCCCACTTGG
AGGCCTTTCATCCCTCGGGTGTGCTATGGATGGCTTCTAACAAAAACTACACATATGTATTCCTGATCGC
CAACCTTTCCCCCACCAGCTAAGGACATTTCCCAGGGTTAATAGGGCCTGGTCCCTGGGAGGAAATTTGA
ATGGGTCCATTTTGCCCTTCCATAGCCTAATCCCTGGGCATTGCTTTCCACTGAGGTTGGGGGTTGGGGT
GTACTAGTTACACATCTTCAACAGACCCCCTCTAGAAATTTTTCAGATGCTTCTGGGAGACACCCAAAGG
GTGAAGCTATTTATCTGTAGTAAACTATTTATCTGTGTTTTTGAAATATTAAACCCTGGATCAGTCCTTT
GATCAGTATAATTTTTTAAAGTTACTTTGTCAGAGGCACAAAAGGGTTTAAACTGATTCATAATAAATAT
CTGTACTTCTTCGATCTTCACCTTTTGTGCTGTGATTCTTCAGTTTCTAAACCAGCACTGTCTGGGTCCC
TACAATGTATCAGGAAGAGCTGAGAATGGTAAGGAGACTCTTCTAAGTCTTCATCTCAGAGACCCTGAGT
TCCCACTCAGACCCACTCAGCCAAATCTCATGGAAGACCAAGGAGGGCAGCACTGTTTTTGTTTTTTGTT
TTTTGTTTTTTTTTTTTGACACTGTCCAAAGGTTTTCCATCCTGTCCTGGAATCAGAGTTGGAAGCTGAG
GAGCTTCAGCCTCTTTTATGGTTTAATGGCCACCTGTTCTCTCCTGTGAAAGGCTTTGCAAAGTCACATT
AAGTTTGCATGACCTGTTATCCCTGGGGCCCTATTTCATAGAGGCTGGCCCTATTAGTGATTTCCAAAAA
CAATATGGAAGTGCCTTTTGATGTCTTACAATAAGAGAAGAAGCCAATGGAAATGAAAGAGATTGGCAAA
GGGGAAGGATGATGCCATGTAGATCCTGTTTGACATTTTTATGGCTGTATTTGTAAACTTAAACACACCA
GTGTCTGTTCTTGATGCAGTTGCTATTTAGGATGAGTTAAGTGCCTGGGGAGTCCCTCAAAAGGTTAAAG
GGATTCCCATCATTGGAATCTTATCACCAGATAGGCAAGTTTATGACCAAACAAGAGAGTACTGGCTTTA
TCCTCTAACCTCATATTTTCTCCCACTTGGCAAGTCCTTTGTGGCATTTATTCATCAGTCAGGGTGTCCG
ATTGGTCCTAGAACTTCCAAAGGCTGCTTGTCATAGAAGCCATTGCATCTATAAAGCAACGGCTCCTGTT
AAATGGTATCTCCTTTCTGAGGCTCCTACTAAAAGTCATTTGTTACCTAAACTTATGTGCTTAACAGGCA
ATGCTTCTCAGACCACAAAGCAGAAAGAAGAAGAAAAGCTCCTGACTAAATCAGGGCTGGGCTTAGACAG
AGTTGATCTGTAGAATATCTTTAAAGGAGAGATGTCAACTTTCTGCACTATTCCCAGCCTCTGCTCCTCC
CTGTCTACCCTCTCCCCTCCCTCTCTCCCTCCACTTCACCCCACAATCTTGAAAAACTTCCTTTCTCTTC
TGTGAACATCATTGGCCAGATCCATTTTCAGTGGTCTGGATTTCTTTTTATTTTCTTTTCAACTTGAAAG
AAACTGGACATTAGGCCACTATGTGTTGTTACTGCCACTAGTGTTCAAGTGCCTCTTGTTTTCCCAGAGA
TTTCCTGGGTCTGCCAGAGGCCCAGACAGGCTCACTCAAGCTCTTTAACTGAAAAGCAACAAGCCACTCC
AGGACAAGGTTCAAAATGGTTACAACAGCCTCTACCTGTCGCCCCAGGGAGAAAGGGGTAGTGATACAAG
TCTCATAGCCAGAGATGGTTTTCCACTCCTTCTAGATATTCCCAAAAAGAGGCTGAGACAGGAGGTTATT
TTCAATTTTATTTTGGAATTAAATACTTTTTTCCCTTTATTACTGTTGTAGTCCCTCACTTGGATATACC
TCTGTTTTCACGATAGAAATAAGGGAGGTCTAGAGCTTCTATTCCTTGGCCATTGTCAACGGAGAGCTGG
CCAAGTCTTCACAAACCCTTGCAACATTGCCTGAAGTTTATGGAATAAGATGTATTCTCACTCCCTTGAT
CTCAAGGGCGTAACTCTGGAAGCACAGCTTGACTACACGTCATTTTTACCAATGATTTTCAGGTGACCTG
GGCTAAGTCATTTAAACTGGGTCTTTATAAAAGTAAAAGGCCAACATTTAATTATTTTGCAAAGCAACCT
AAGAGCTAAAGATGTAATTTTTCTTGCAATTGTAAATCTTTTGTGTCTCCTGAAGACTTCCCTTAAAATT
AGCTCTGAGTGAAAAATCAAAAGAGACAAAAGACATCTTCGAATCCATATTTCAAGCCTGGTAGAATTGG
CTTTTCTAGCAGAACCTTTCCAAAAGTTTTATATTGAGATTCATAACAACACCAAGAATTGATTTTGTAG
CCAACATTCATTCAATACTGTTATATCAGAGGAGTAGGAGAGAGGAAACATTTGACTTATCTGGAAAAGC
AAAATGTACTTAAGAATAAGAATAACATGGTCCATTCACCTTTATGTTATAGATATGTCTTTGTGTAAAT
CATTTGTTTTGAGTTTTCAAAGAATAGCCCATTGTTCATTCTTGTGCTGTACAATGACCACTGTTATTGT
TACTTTGACTTTTCAGAGCACACCCTTCCTCTGGTTTTTGTATATTTATTGATGGATCAATAATAATGAG
GAAAGCATGATATGTATATTGCTGAGTTGAAAGCACTTATTGGAAAATATTAAAAGGCTAACATTAAAAG
ACTAAAGGAAACAGAAAAAAAAAAAAAAAAA
>gi|48255940|ref|NM_001001391.1| Homo sapiens CD44 molecule (Indian
blood group) (CD44), transcript variant 4, mRNA (SEQ ID NO: 14)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGGTATGGGTTCATAGAAGGGCACGTGGTGATTCC
CCGGATCCACCCCAACTCCATCTGTGCAGCAAACAACACAGGGGTGTACATCCTCACATCCAACACCTCC
CAGTATGACACATATTGCTTCAATGCTTCAGCTCCACCTGAAGAAGATTGTACATCAGTCACAGACCTGC
CCAATGCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCGGCTCC
TCCAGTGAAAGGAGCAGCACTTCAGGAGGTTACATCTTTTACACCTTTTCTACTGTACACCCCATCCCAG
ACGAAGACAGTCCCTGGATCACCGACAGCACAGACAGAATCCCTGCTACCAGAGACCAAGACACATTCCA
CCCCAGTGGGGGGTCCCATACCACTCATGGATCTGAATCAGATGGACACTCACATGGGAGTCAAGAAGGT
GGAGCAAACACAACCTCTGGTCCTATAAGGACACCCCAAATTCCAGAATGGCTGATCATCTTGGCATCCC
TCTTGGCCTTGGCTTTGATTCTTGCAGTTTGCATTGCAGTCAACAGTCGAAGAAGGTGTGGGCAGAAGAA
AAAGCTAGTGATCAACAGTGGCAATGGAGCTGTGGAGGACAGAAAGCCAAGTGGACTCAACGGAGAGGCC
AGCAAGTCTCAGGAAATGGTGCATTTGGTGAACAAGGAGTCGTCAGAAACTCCAGACCAGTTTATGACAG
CTGATGAGACAAGGAACCTGCAGAATGTGGACATGAAGATTGGGGTGTAACACCTACACCATTATCTTGG
AAAGAAACAACCGTTGGAAACATAACCATTACAGGGAGCTGGGACACTTAACAGATGCAATGTGCTACTG
ATTGTTTCATTGCGAATCTTTTTTAGCATAAAATTTTCTACTCTTTTTGTTTTTTGTGTTTTGTTCTTTA
AAGTCAGGTCCAATTTGTAAAAACAGCATTGCTTTCTGAAATTAGGGCCCAATTAATAATCAGCAAGAAT
TTGATCGTTCCAGTTCCCACTTGGAGGCCTTTCATCCCTCGGGTGTGCTATGGATGGCTTCTAACAAAAA
CTACACATATGTATTCCTGATCGCCAACCTTTCCCCCACCAGCTAAGGACATTTCCCAGGGTTAATAGGG
CCTGGTCCCTGGGAGGAAATTTGAATGGGTCCATTTTGCCCTTCCATAGCCTAATCCCTGGGCATTGCTT
TCCACTGAGGTTGGGGGTTGGGGTGTACTAGTTACACATCTTCAACAGACCCCCTCTAGAAATTTTTCAG
ATGCTTCTGGGAGACACCCAAAGGGTGAAGCTATTTATCTGTAGTAAACTATTTATCTGTGTTTTTGAAA
TATTAAACCCTGGATCAGTCCTTTGATCAGTATAATTTTTTAAAGTTACTTTGTCAGAGGCACAAAAGGG
TTTAAACTGATTCATAATAAATATCTGTACTTCTTCGATCTTCACCTTTTGTGCTGTGATTCTTCAGTTT
CTAAACCAGCACTGTCTGGGTCCCTACAATGTATCAGGAAGAGCTGAGAATGGTAAGGAGACTCTTCTAA
GTCTTCATCTCAGAGACCCTGAGTTCCCACTCAGACCCACTCAGCCAAATCTCATGGAAGACCAAGGAGG
GCAGCACTGTTTTTGTTTTTTGTTTTTTGTTTTTTTTTTTTGACACTGTCCAAAGGTTTTCCATCCTGTC
CTGGAATCAGAGTTGGAAGCTGAGGAGCTTCAGCCTCTTTTATGGTTTAATGGCCACCTGTTCTCTCCTG
TGAAAGGCTTTGCAAAGTCACATTAAGTTTGCATGACCTGTTATCCCTGGGGCCCTATTTCATAGAGGCT
GGCCCTATTAGTGATTTCCAAAAACAATATGGAAGTGCCTTTTGATGTCTTACAATAAGAGAAGAAGCCA
ATGGAAATGAAAGAGATTGGCAAAGGGGAAGGATGATGCCATGTAGATCCTGTTTGACATTTTTATGGCT
GTATTTGTAAACTTAAACACACCAGTGTCTGTTCTTGATGCAGTTGCTATTTAGGATGAGTTAAGTGCCT
GGGGAGTCCCTCAAAAGGTTAAAGGGATTCCCATCATTGGAATCTTATCACCAGATAGGCAAGTTTATGA
CCAAACAAGAGAGTACTGGCTTTATCCTCTAACCTCATATTTTCTCCCACTTGGCAAGTCCTTTGTGGCA
TTTATTCATCAGTCAGGGTGTCCGATTGGTCCTAGAACTTCCAAAGGCTGCTTGTCATAGAAGCCATTGC
ATCTATAAAGCAACGGCTCCTGTTAAATGGTATCTCCTTTCTGAGGCTCCTACTAAAAGTCATTTGTTAC
CTAAACTTATGTGCTTAACAGGCAATGCTTCTCAGACCACAAAGCAGAAAGAAGAAGAAAAGCTCCTGAC
TAAATCAGGGCTGGGCTTAGACAGAGTTGATCTGTAGAATATCTTTAAAGGAGAGATGTCAACTTTCTGC
ACTATTCCCAGCCTCTGCTCCTCCCTGTCTACCCTCTCCCCTCCCTCTCTCCCTCCACTTCACCCCACAA
TCTTGAAAAACTTCCTTTCTCTTCTGTGAACATCATTGGCCAGATCCATTTTCAGTGGTCTGGATTTCTT
TTTATTTTCTTTTCAACTTGAAAGAAACTGGACATTAGGCCACTATGTGTTGTTACTGCCACTAGTGTTC
AAGTGCCTCTTGTTTTCCCAGAGATTTCCTGGGTCTGCCAGAGGCCCAGACAGGCTCACTCAAGCTCTTT
AACTGAAAAGCAACAAGCCACTCCAGGACAAGGTTCAAAATGGTTACAACAGCCTCTACCTGTCGCCCCA
GGGAGAAAGGGGTAGTGATACAAGTCTCATAGCCAGAGATGGTTTTCCACTCCTTCTAGATATTCCCAAA
AAGAGGCTGAGACAGGAGGTTATTTTCAATTTTATTTTGGAATTAAATACTTTTTTCCCTTTATTACTGT
TGTAGTCCCTCACTTGGATATACCTCTGTTTTCACGATAGAAATAAGGGAGGTCTAGAGCTTCTATTCCT
TGGCCATTGTCAACGGAGAGCTGGCCAAGTCTTCACAAACCCTTGCAACATTGCCTGAAGTTTATGGAAT
AAGATGTATTCTCACTCCCTTGATCTCAAGGGCGTAACTCTGGAAGCACAGCTTGACTACACGTCATTTT
TACCAATGATTTTCAGGTGACCTGGGCTAAGTCATTTAAACTGGGTCTTTATAAAAGTAAAAGGCCAACA
TTTAATTATTTTGCAAAGCAACCTAAGAGCTAAAGATGTAATTTTTCTTGCAATTGTAAATCTTTTGTGT
CTCCTGAAGACTTCCCTTAAAATTAGCTCTGAGTGAAAAATCAAAAGAGACAAAAGACATCTTCGAATCC
ATATTTCAAGCCTGGTAGAATTGGCTTTTCTAGCAGAACCTTTCCAAAAGTTTTATATTGAGATTCATAA
CAACACCAAGAATTGATTTTGTAGCCAACATTCATTCAATACTGTTATATCAGAGGAGTAGGAGAGAGGA
AACATTTGACTTATCTGGAAAAGCAAAATGTACTTAAGAATAAGAATAACATGGTCCATTCACCTTTATG
TTATAGATATGTCTTTGTGTAAATCATTTGTTTTGAGTTTTCAAAGAATAGCCCATTGTTCATTCTTGTG
CTGTACAATGACCACTGTTATTGTTACTTTGACTTTTCAGAGCACACCCTTCCTCTGGTTTTTGTATATT
TATTGATGGATCAATAATAATGAGGAAAGCATGATATGTATATTGCTGAGTTGAAAGCACTTATTGGAAA
ATATTAAAAGGCTAACATTAAAAGACTAAAGGAAACAGAAAAAAAAAAAAAAAAA
>gi|48255942|ref|NM_001001392.1| Homo sapiens CD44 molecule (Indian
blood group) (CD44), transcript variant 5, mRNA (SEQ ID NO: 15)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGTTTGCATTGCAGTCAACAGTCGAAGAAGGTGTG
GGCAGAAGAAAAAGCTAGTGATCAACAGTGGCAATGGAGCTGTGGAGGACAGAAAGCCAAGTGGACTCAA
CGGAGAGGCCAGCAAGTCTCAGGAAATGGTGCATTTGGTGAACAAGGAGTCGTCAGAAACTCCAGACCAG
TTTATGACAGCTGATGAGACAAGGAACCTGCAGAATGTGGACATGAAGATTGGGGTGTAACACCTACACC
ATTATCTTGGAAAGAAACAACCGTTGGAAACATAACCATTACAGGGAGCTGGGACACTTAACAGATGCAA
TGTGCTACTGATTGTTTCATTGCGAATCTTTTTTAGCATAAAATTTTCTACTCTTTTTGTTTTTTGTGTT
TTGTTCTTTAAAGTCAGGTCCAATTTGTAAAAACAGCATTGCTTTCTGAAATTAGGGCCCAATTAATAAT
CAGCAAGAATTTGATCGTTCCAGTTCCCACTTGGAGGCCTTTCATCCCTCGGGTGTGCTATGGATGGCTT
CTAACAAAAACTACACATATGTATTCCTGATCGCCAACCTTTCCCCCACCAGCTAAGGACATTTCCCAGG
GTTAATAGGGCCTGGTCCCTGGGAGGAAATTTGAATGGGTCCATTTTGCCCTTCCATAGCCTAATCCCTG
GGCATTGCTTTCCACTGAGGTTGGGGGTTGGGGTGTACTAGTTACACATCTTCAACAGACCCCCTCTAGA
AATTTTTCAGATGCTTCTGGGAGACACCCAAAGGGTGAAGCTATTTATCTGTAGTAAACTATTTATCTGT
GTTTTTGAAATATTAAACCCTGGATCAGTCCTTTGATCAGTATAATTTTTTAAAGTTACTTTGTCAGAGG
CACAAAAGGGTTTAAACTGATTCATAATAAATATCTGTACTTCTTCGATCTTCACCTTTTGTGCTGTGAT
TCTTCAGTTTCTAAACCAGCACTGTCTGGGTCCCTACAATGTATCAGGAAGAGCTGAGAATGGTAAGGAG
ACTCTTCTAAGTCTTCATCTCAGAGACCCTGAGTTCCCACTCAGACCCACTCAGCCAAATCTCATGGAAG
ACCAAGGAGGGCAGCACTGTTTTTGTTTTTTGTTTTTTGTTTTTTTTTTTTGACACTGTCCAAAGGTTTT
CCATCCTGTCCTGGAATCAGAGTTGGAAGCTGAGGAGCTTCAGCCTCTTTTATGGTTTAATGGCCACCTG
TTCTCTCCTGTGAAAGGCTTTGCAAAGTCACATTAAGTTTGCATGACCTGTTATCCCTGGGGCCCTATTT
CATAGAGGCTGGCCCTATTAGTGATTTCCAAAAACAATATGGAAGTGCCTTTTGATGTCTTACAATAAGA
GAAGAAGCCAATGGAAATGAAAGAGATTGGCAAAGGGGAAGGATGATGCCATGTAGATCCTGTTTGACAT
TTTTATGGCTGTATTTGTAAACTTAAACACACCAGTGTCTGTTCTTGATGCAGTTGCTATTTAGGATGAG
TTAAGTGCCTGGGGAGTCCCTCAAAAGGTTAAAGGGATTCCCATCATTGGAATCTTATCACCAGATAGGC
AAGTTTATGACCAAACAAGAGAGTACTGGCTTTATCCTCTAACCTCATATTTTCTCCCACTTGGCAAGTC
CTTTGTGGCATTTATTCATCAGTCAGGGTGTCCGATTGGTCCTAGAACTTCCAAAGGCTGCTTGTCATAG
AAGCCATTGCATCTATAAAGCAACGGCTCCTGTTAAATGGTATCTCCTTTCTGAGGCTCCTACTAAAAGT
CATTTGTTACCTAAACTTATGTGCTTAACAGGCAATGCTTCTCAGACCACAAAGCAGAAAGAAGAAGAAA
AGCTCCTGACTAAATCAGGGCTGGGCTTAGACAGAGTTGATCTGTAGAATATCTTTAAAGGAGAGATGTC
AACTTTCTGCACTATTCCCAGCCTCTGCTCCTCCCTGTCTACCCTCTCCCCTCCCTCTCTCCCTCCACTT
CACCCCACAATCTTGAAAAACTTCCTTTCTCTTCTGTGAACATCATTGGCCAGATCCATTTTCAGTGGTC
TGGATTTCTTTTTATTTTCTTTTCAACTTGAAAGAAACTGGACATTAGGCCACTATGTGTTGTTACTGCC
ACTAGTGTTCAAGTGCCTCTTGTTTTCCCAGAGATTTCCTGGGTCTGCCAGAGGCCCAGACAGGCTCACT
CAAGCTCTTTAACTGAAAAGCAACAAGCCACTCCAGGACAAGGTTCAAAATGGTTACAACAGCCTCTACC
TGTCGCCCCAGGGAGAAAGGGGTAGTGATACAAGTCTCATAGCCAGAGATGGTTTTCCACTCCTTCTAGA
TATTCCCAAAAAGAGGCTGAGACAGGAGGTTATTTTCAATTTTATTTTGGAATTAAATACTTTTTTCCCT
TTATTACTGTTGTAGTCCCTCACTTGGATATACCTCTGTTTTCACGATAGAAATAAGGGAGGTCTAGAGC
TTCTATTCCTTGGCCATTGTCAACGGAGAGCTGGCCAAGTCTTCACAAACCCTTGCAACATTGCCTGAAG
TTTATGGAATAAGATGTATTCTCACTCCCTTGATCTCAAGGGCGTAACTCTGGAAGCACAGCTTGACTAC
ACGTCATTTTTACCAATGATTTTCAGGTGACCTGGGCTAAGTCATTTAAACTGGGTCTTTATAAAAGTAA
AAGGCCAACATTTAATTATTTTGCAAAGCAACCTAAGAGCTAAAGATGTAATTTTTCTTGCAATTGTAAA
TCTTTTGTGTCTCCTGAAGACTTCCCTTAAAATTAGCTCTGAGTGAAAAATCAAAAGAGACAAAAGACAT
CTTCGAATCCATATTTCAAGCCTGGTAGAATTGGCTTTTCTAGCAGAACCTTTCCAAAAGTTTTATATTG
AGATTCATAACAACACCAAGAATTGATTTTGTAGCCAACATTCATTCAATACTGTTATATCAGAGGAGTA
GGAGAGAGGAAACATTTGACTTATCTGGAAAAGCAAAATGTACTTAAGAATAAGAATAACATGGTCCATT
CACCTTTATGTTATAGATATGTCTTTGTGTAAATCATTTGTTTTGAGTTTTCAAAGAATAGCCCATTGTT
CATTCTTGTGCTGTACAATGACCACTGTTATTGTTACTTTGACTTTTCAGAGCACACCCTTCCTCTGGTT
TTTGTATATTTATTGATGGATCAATAATAATGAGGAAAGCATGATATGTATATTGCTGAGTTGAAAGCAC
TTATTGGAAAATATTAAAAGGCTAACATTAAAAGACTAAAGGAAACAGAAAAAAAAAAAAAAAAA
>gi|321400137|ref|NM_001202555.1| Homo sapiens CD44 molecule (Indian
blood group) (CD44), transcript variant 6, mRNA (SEQ ID NO: 16)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGGTATGGGTTCATAGAAGGGCACGTGGTGATTCC
CCGGATCCACCCCAACTCCATCTGTGCAGCAAACAACACAGGGGTGTACATCCTCACATCCAACACCTCC
CAGTATGACACATATTGCTTCAATGCTTCAGCTCCACCTGAAGAAGATTGTACATCAGTCACAGACCTGC
CCAATGCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCGGCTCC
TCCAGTGAAAGGAGCAGCACTTCAGGAGGTTACATCTTTTACACCTTTTCTACTGTACACCCCATCCCAG
ACGAAGACAGTCCCTGGATCACCGACAGCACAGACAGAATCCCTGCTACCAATAGGAATGATGTCACAGG
TGGAAGAAGAGACCCAAATCATTCTGAAGGCTCAACTACTTTACTGGAAGGTTATACCTCTCATTACCCA
CACACGAAGGAAAGCAGGACCTTCATCCCAGTGACCTCAGCTAAGACTGGGTCCTTTGGAGTTACTGCAG
TTACTGTTGGAGATTCCAACTCTAATGTCAATCGTTCCTTATCAGGAGACCAAGACACATTCCACCCCAG
TGGGGGGTCCCATACCACTCATGGATCTGAATCAGATGGACACTCACATGGGAGTCAAGAAGGTGGAGCA
AACACAACCTCTGGTCCTATAAGGACACCCCAAATTCCAGAATGGCTGATCATCTTGGCATCCCTCTTGG
CCTTGGCTTTGATTCTTGCAGTTTGCATTGCAGTCAACAGTCGAAGAAGGTGTGGGCAGAAGAAAAAGCT
AGTGATCAACAGTGGCAATGGAGCTGTGGAGGACAGAAAGCCAAGTGGACTCAACGGAGAGGCCAGCAAG
TCTCAGGAAATGGTGCATTTGGTGAACAAGGAGTCGTCAGAAACTCCAGACCAGTTTATGACAGCTGATG
AGACAAGGAACCTGCAGAATGTGGACATGAAGATTGGGGTGTAACACCTACACCATTATCTTGGAAAGAA
ACAACCGTTGGAAACATAACCATTACAGGGAGCTGGGACACTTAACAGATGCAATGTGCTACTGATTGTT
TCATTGCGAATCTTTTTTAGCATAAAATTTTCTACTCTTTTTGTTTTTTGTGTTTTGTTCTTTAAAGTCA
GGTCCAATTTGTAAAAACAGCATTGCTTTCTGAAATTAGGGCCCAATTAATAATCAGCAAGAATTTGATC
GTTCCAGTTCCCACTTGGAGGCCTTTCATCCCTCGGGTGTGCTATGGATGGCTTCTAACAAAAACTACAC
ATATGTATTCCTGATCGCCAACCTTTCCCCCACCAGCTAAGGACATTTCCCAGGGTTAATAGGGCCTGGT
CCCTGGGAGGAAATTTGAATGGGTCCATTTTGCCCTTCCATAGCCTAATCCCTGGGCATTGCTTTCCACT
GAGGTTGGGGGTTGGGGTGTACTAGTTACACATCTTCAACAGACCCCCTCTAGAAATTTTTCAGATGCTT
CTGGGAGACACCCAAAGGGTGAAGCTATTTATCTGTAGTAAACTATTTATCTGTGTTTTTGAAATATTAA
ACCCTGGATCAGTCCTTTGATCAGTATAATTTTTTAAAGTTACTTTGTCAGAGGCACAAAAGGGTTTAAA
CTGATTCATAATAAATATCTGTACTTCTTCGATCTTCACCTTTTGTGCTGTGATTCTTCAGTTTCTAAAC
CAGCACTGTCTGGGTCCCTACAATGTATCAGGAAGAGCTGAGAATGGTAAGGAGACTCTTCTAAGTCTTC
ATCTCAGAGACCCTGAGTTCCCACTCAGACCCACTCAGCCAAATCTCATGGAAGACCAAGGAGGGCAGCA
CTGTTTTTGTTTTTTGTTTTTTGTTTTTTTTTTTTGACACTGTCCAAAGGTTTTCCATCCTGTCCTGGAA
TCAGAGTTGGAAGCTGAGGAGCTTCAGCCTCTTTTATGGTTTAATGGCCACCTGTTCTCTCCTGTGAAAG
GCTTTGCAAAGTCACATTAAGTTTGCATGACCTGTTATCCCTGGGGCCCTATTTCATAGAGGCTGGCCCT
ATTAGTGATTTCCAAAAACAATATGGAAGTGCCTTTTGATGTCTTACAATAAGAGAAGAAGCCAATGGAA
ATGAAAGAGATTGGCAAAGGGGAAGGATGATGCCATGTAGATCCTGTTTGACATTTTTATGGCTGTATTT
GTAAACTTAAACACACCAGTGTCTGTTCTTGATGCAGTTGCTATTTAGGATGAGTTAAGTGCCTGGGGAG
TCCCTCAAAAGGTTAAAGGGATTCCCATCATTGGAATCTTATCACCAGATAGGCAAGTTTATGACCAAAC
AAGAGAGTACTGGCTTTATCCTCTAACCTCATATTTTCTCCCACTTGGCAAGTCCTTTGTGGCATTTATT
CATCAGTCAGGGTGTCCGATTGGTCCTAGAACTTCCAAAGGCTGCTTGTCATAGAAGCCATTGCATCTAT
AAAGCAACGGCTCCTGTTAAATGGTATCTCCTTTCTGAGGCTCCTACTAAAAGTCATTTGTTACCTAAAC
TTATGTGCTTAACAGGCAATGCTTCTCAGACCACAAAGCAGAAAGAAGAAGAAAAGCTCCTGACTAAATC
AGGGCTGGGCTTAGACAGAGTTGATCTGTAGAATATCTTTAAAGGAGAGATGTCAACTTTCTGCACTATT
CCCAGCCTCTGCTCCTCCCTGTCTACCCTCTCCCCTCCCTCTCTCCCTCCACTTCACCCCACAATCTTGA
AAAACTTCCTTTCTCTTCTGTGAACATCATTGGCCAGATCCATTTTCAGTGGTCTGGATTTCTTTTTATT
TTCTTTTCAACTTGAAAGAAACTGGACATTAGGCCACTATGTGTTGTTACTGCCACTAGTGTTCAAGTGC
CTCTTGTTTTCCCAGAGATTTCCTGGGTCTGCCAGAGGCCCAGACAGGCTCACTCAAGCTCTTTAACTGA
AAAGCAACAAGCCACTCCAGGACAAGGTTCAAAATGGTTACAACAGCCTCTACCTGTCGCCCCAGGGAGA
AAGGGGTAGTGATACAAGTCTCATAGCCAGAGATGGTTTTCCACTCCTTCTAGATATTCCCAAAAAGAGG
CTGAGACAGGAGGTTATTTTCAATTTTATTTTGGAATTAAATACTTTTTTCCCTTTATTACTGTTGTAGT
CCCTCACTTGGATATACCTCTGTTTTCACGATAGAAATAAGGGAGGTCTAGAGCTTCTATTCCTTGGCCA
TTGTCAACGGAGAGCTGGCCAAGTCTTCACAAACCCTTGCAACATTGCCTGAAGTTTATGGAATAAGATG
TATTCTCACTCCCTTGATCTCAAGGGCGTAACTCTGGAAGCACAGCTTGACTACACGTCATTTTTACCAA
TGATTTTCAGGTGACCTGGGCTAAGTCATTTAAACTGGGTCTTTATAAAAGTAAAAGGCCAACATTTAAT
TATTTTGCAAAGCAACCTAAGAGCTAAAGATGTAATTTTTCTTGCAATTGTAAATCTTTTGTGTCTCCTG
AAGACTTCCCTTAAAATTAGCTCTGAGTGAAAAATCAAAAGAGACAAAAGACATCTTCGAATCCATATTT
CAAGCCTGGTAGAATTGGCTTTTCTAGCAGAACCTTTCCAAAAGTTTTATATTGAGATTCATAACAACAC
CAAGAATTGATTTTGTAGCCAACATTCATTCAATACTGTTATATCAGAGGAGTAGGAGAGAGGAAACATT
TGACTTATCTGGAAAAGCAAAATGTACTTAAGAATAAGAATAACATGGTCCATTCACCTTTATGTTATAG
ATATGTCTTTGTGTAAATCATTTGTTTTGAGTTTTCAAAGAATAGCCCATTGTTCATTCTTGTGCTGTAC
AATGACCACTGTTATTGTTACTTTGACTTTTCAGAGCACACCCTTCCTCTGGTTTTTGTATATTTATTGA
TGGATCAATAATAATGAGGAAAGCATGATATGTATATTGCTGAGTTGAAAGCACTTATTGGAAAATATTA
AAAGGCTAACATTAAAAGACTAAAGGAAACAGAAAAAAAAAAAAAAAAA
>gi|321400139|ref|NM_001202556.1| Homo sapiens CD44 molecule (Indian
blood group) (CD44), transcript variant 7, mRNA (SEQ ID NO: 17)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGGTATGGGTTCATAGAAGGGCACGTGGTGATTCC
CCGGATCCACCCCAACTCCATCTGTGCAGCAAACAACACAGGGGTGTACATCCTCACATCCAACACCTCC
CAGTATGACACATATTGCTTCAATGCTTCAGCTCCACCTGAAGAAGATTGTACATCAGTCACAGACCTGC
CCAATGCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCGGCTCC
TCCAGTGAAAGGAGCAGCACTTCAGGAGGTTACATCTTTTACACCTTTTCTACTGTACACCCCATCCCAG
ACGAAGACAGTCCCTGGATCACCGACAGCACAGACAGAATCCCTGCTACCAGACACTCACATGGGAGTCA
AGAAGGTGGAGCAAACACAACCTCTGGTCCTATAAGGACACCCCAAATTCCAGAATGGCTGATCATCTTG
GCATCCCTCTTGGCCTTGGCTTTGATTCTTGCAGTTTGCATTGCAGTCAACAGTCGAAGAAGGTGTGGGC
AGAAGAAAAAGCTAGTGATCAACAGTGGCAATGGAGCTGTGGAGGACAGAAAGCCAAGTGGACTCAACGG
AGAGGCCAGCAAGTCTCAGGAAATGGTGCATTTGGTGAACAAGGAGTCGTCAGAAACTCCAGACCAGTTT
ATGACAGCTGATGAGACAAGGAACCTGCAGAATGTGGACATGAAGATTGGGGTGTAACACCTACACCATT
ATCTTGGAAAGAAACAACCGTTGGAAACATAACCATTACAGGGAGCTGGGACACTTAACAGATGCAATGT
GCTACTGATTGTTTCATTGCGAATCTTTTTTAGCATAAAATTTTCTACTCTTTTTGTTTTTTGTGTTTTG
TTCTTTAAAGTCAGGTCCAATTTGTAAAAACAGCATTGCTTTCTGAAATTAGGGCCCAATTAATAATCAG
CAAGAATTTGATCGTTCCAGTTCCCACTTGGAGGCCTTTCATCCCTCGGGTGTGCTATGGATGGCTTCTA
ACAAAAACTACACATATGTATTCCTGATCGCCAACCTTTCCCCCACCAGCTAAGGACATTTCCCAGGGTT
AATAGGGCCTGGTCCCTGGGAGGAAATTTGAATGGGTCCATTTTGCCCTTCCATAGCCTAATCCCTGGGC
ATTGCTTTCCACTGAGGTTGGGGGTTGGGGTGTACTAGTTACACATCTTCAACAGACCCCCTCTAGAAAT
TTTTCAGATGCTTCTGGGAGACACCCAAAGGGTGAAGCTATTTATCTGTAGTAAACTATTTATCTGTGTT
TTTGAAATATTAAACCCTGGATCAGTCCTTTGATCAGTATAATTTTTTAAAGTTACTTTGTCAGAGGCAC
AAAAGGGTTTAAACTGATTCATAATAAATATCTGTACTTCTTCGATCTTCACCTTTTGTGCTGTGATTCT
TCAGTTTCTAAACCAGCACTGTCTGGGTCCCTACAATGTATCAGGAAGAGCTGAGAATGGTAAGGAGACT
CTTCTAAGTCTTCATCTCAGAGACCCTGAGTTCCCACTCAGACCCACTCAGCCAAATCTCATGGAAGACC
AAGGAGGGCAGCACTGTTTTTGTTTTTTGTTTTTTGTTTTTTTTTTTTGACACTGTCCAAAGGTTTTCCA
TCCTGTCCTGGAATCAGAGTTGGAAGCTGAGGAGCTTCAGCCTCTTTTATGGTTTAATGGCCACCTGTTC
TCTCCTGTGAAAGGCTTTGCAAAGTCACATTAAGTTTGCATGACCTGTTATCCCTGGGGCCCTATTTCAT
AGAGGCTGGCCCTATTAGTGATTTCCAAAAACAATATGGAAGTGCCTTTTGATGTCTTACAATAAGAGAA
GAAGCCAATGGAAATGAAAGAGATTGGCAAAGGGGAAGGATGATGCCATGTAGATCCTGTTTGACATTTT
TATGGCTGTATTTGTAAACTTAAACACACCAGTGTCTGTTCTTGATGCAGTTGCTATTTAGGATGAGTTA
AGTGCCTGGGGAGTCCCTCAAAAGGTTAAAGGGATTCCCATCATTGGAATCTTATCACCAGATAGGCAAG
TTTATGACCAAACAAGAGAGTACTGGCTTTATCCTCTAACCTCATATTTTCTCCCACTTGGCAAGTCCTT
TGTGGCATTTATTCATCAGTCAGGGTGTCCGATTGGTCCTAGAACTTCCAAAGGCTGCTTGTCATAGAAG
CCATTGCATCTATAAAGCAACGGCTCCTGTTAAATGGTATCTCCTTTCTGAGGCTCCTACTAAAAGTCAT
TTGTTACCTAAACTTATGTGCTTAACAGGCAATGCTTCTCAGACCACAAAGCAGAAAGAAGAAGAAAAGC
TCCTGACTAAATCAGGGCTGGGCTTAGACAGAGTTGATCTGTAGAATATCTTTAAAGGAGAGATGTCAAC
TTTCTGCACTATTCCCAGCCTCTGCTCCTCCCTGTCTACCCTCTCCCCTCCCTCTCTCCCTCCACTTCAC
CCCACAATCTTGAAAAACTTCCTTTCTCTTCTGTGAACATCATTGGCCAGATCCATTTTCAGTGGTCTGG
ATTTCTTTTTATTTTCTTTTCAACTTGAAAGAAACTGGACATTAGGCCACTATGTGTTGTTACTGCCACT
AGTGTTCAAGTGCCTCTTGTTTTCCCAGAGATTTCCTGGGTCTGCCAGAGGCCCAGACAGGCTCACTCAA
GCTCTTTAACTGAAAAGCAACAAGCCACTCCAGGACAAGGTTCAAAATGGTTACAACAGCCTCTACCTGT
CGCCCCAGGGAGAAAGGGGTAGTGATACAAGTCTCATAGCCAGAGATGGTTTTCCACTCCTTCTAGATAT
TCCCAAAAAGAGGCTGAGACAGGAGGTTATTTTCAATTTTATTTTGGAATTAAATACTTTTTTCCCTTTA
TTACTGTTGTAGTCCCTCACTTGGATATACCTCTGTTTTCACGATAGAAATAAGGGAGGTCTAGAGCTTC
TATTCCTTGGCCATTGTCAACGGAGAGCTGGCCAAGTCTTCACAAACCCTTGCAACATTGCCTGAAGTTT
ATGGAATAAGATGTATTCTCACTCCCTTGATCTCAAGGGCGTAACTCTGGAAGCACAGCTTGACTACACG
TCATTTTTACCAATGATTTTCAGGTGACCTGGGCTAAGTCATTTAAACTGGGTCTTTATAAAAGTAAAAG
GCCAACATTTAATTATTTTGCAAAGCAACCTAAGAGCTAAAGATGTAATTTTTCTTGCAATTGTAAATCT
TTTGTGTCTCCTGAAGACTTCCCTTAAAATTAGCTCTGAGTGAAAAATCAAAAGAGACAAAAGACATCTT
CGAATCCATATTTCAAGCCTGGTAGAATTGGCTTTTCTAGCAGAACCTTTCCAAAAGTTTTATATTGAGA
TTCATAACAACACCAAGAATTGATTTTGTAGCCAACATTCATTCAATACTGTTATATCAGAGGAGTAGGA
GAGAGGAAACATTTGACTTATCTGGAAAAGCAAAATGTACTTAAGAATAAGAATAACATGGTCCATTCAC
CTTTATGTTATAGATATGTCTTTGTGTAAATCATTTGTTTTGAGTTTTCAAAGAATAGCCCATTGTTCAT
TCTTGTGCTGTACAATGACCACTGTTATTGTTACTTTGACTTTTCAGAGCACACCCTTCCTCTGGTTTTT
GTATATTTATTGATGGATCAATAATAATGAGGAAAGCATGATATGTATATTGCTGAGTTGAAAGCACTTA
TTGGAAAATATTAAAAGGCTAACATTAAAAGACTAAAGGAAACAGAAAAAAAAAAAAAAAAA
>gi|321400141|ref|NM_001202557.1| Homo sapiens CD44 molecule (Indian
blood group) (CD44), transcript variant 8, mRNA (SEQ ID NO: 18)
GAGAAGAAAGCCAGTGCGTCTCTGGGCGCAGGGGCCAGTGGGGCTCGGAGGCACAGGCACCCCGCGACAC
TCCAGGTTCCCCGACCCACGTCCCTGGCAGCCCCGATTATTTACAGCCTCAGCAGAGCACGGGGCGGGGG
CAGAGGGGCCCGCCCGGGAGGGCTGCTACTTCTTAAAACCTCTGCGGGCTGCTTAGTCACAGCCCCCCTT
GCTTGGGTGTGTCCTTCGCTCGCTCCCTCCCTCCGTCTTAGGTCACTGTTTTCAACCTCGAATAAAAACT
GCAGCCAACTTCCGAGGCAGCCTCATTGCCCAGCGGACCCCAGCCTCTGCCAGGTTCGGTCCGCCATCCT
CGTCCCGTCCTCCGCCGGCCCCTGCCCCGCGCCCAGGGATCCTCCAGCTCCTTTCGCCCGCGCCCTCCGT
TCGCTCCGGACACCATGGACAAGTTTTGGTGGCACGCAGCCTGGGGACTCTGCCTCGTGCCGCTGAGCCT
GGCGCAGATCGATTTGAATATAACCTGCCGCTTTGCAGGTGTATTCCACGTGGAGAAAAATGGTCGCTAC
AGCATCTCTCGGACGGAGGCCGCTGACCTCTGCAAGGCTTTCAATAGCACCTTGCCCACAATGGCCCAGA
TGGAGAAAGCTCTGAGCATCGGATTTGAGACCTGCAGGTATGGGTTCATAGAAGGGCACGTGGTGATTCC
CCGGATCCACCCCAACTCCATCTGTGCAGCAAACAACACAGGGGTGTACATCCTCACATCCAACACCTCC
CAGTATGACACATATTGCTTCAATGCTTCAGCTCCACCTGAAGAAGATTGTACATCAGTCACAGACCTGC
CCAATGCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCGGCTCC
TCCAGTGAAAGGAGCAGCACTTCAGGAGGTTACATCTTTTACACCTTTTCTACTGTACACCCCATCCCAG
ACGAAGACAGTCCCTGGATCACCGACAGCACAGACAGAATCCCTGCTACCAGAGACCAAGACACATTCCA
CCCCAGTGGGGGGTCCCATACCACTCATGGATCTGAATCAGATGGACACTCACATGGGAGTCAAGAAGGT
GGAGCAAACACAACCTCTGGTCCTATAAGGACACCCCAAATTCCAGAATGGCTGATCATCTTGGCATCCC
TCTTGGCCTTGGCTTTGATTCTTGCAGTTTGCATTGCAGTCAACAGTCGAAGAAGTTGAAGAGATTCAGG
TTATAGCATAAGAAGAGCACTGTTTCATCGTCTTCTTGCTGTTAGGAGGTCTATGAAGCAGAGAAGAACT
TTCCTTTGGAAAACAACTAAATGAAGACAGTCACCTCGCTAGAACTGACACATGGGCTGTTTTTATATTC
TTGAAGGCCACTCTCTCCCTACCTGAACCAAGACCTATAGGTTTACATGTTATTTACATTTTATATATAA
TATATATATATATATATACACATACATTATATATACACAATAGTAATTCTAGCAACAGAGGAAATGACCT
TTAACAGGGGTATAAATCTAAATTTATAAAAGTATAAATCTAAATTTCTTACCCAAGACACTTTAAAGAT
ACATTATTTTTCTCCAGGACGTAATTCATAGGAATATTAAGCCTTTTGTAAATGTCCCTTTAGATGGTTT
CTCATAAGGTAAAAGAAACTTATTTCCAAGCAGGACCACCTTTATTGTGTCCCCAGATCACCTCACAGGG
CAGAAAAATGCCCCTCAGTCTGGGAGAAGACCTAGAGAGAATTATGGACTCCTTACTGGTTTTTGGAAAG
CAACCAACAGCTAATTCCAACACCATGGGCAGCCCATACAGTCTCTAATTATCTGAGAAAATCAAATGAT
GCTGTTACAATAATTACGCTGGTACAAGTTAATAAAAGTGCCATGTTACAGTCAAACAGCTATGTTGCTA
TCTATACCATTGAGGGCATAGTTTTAAAAAGTAGTTATGCTACCTGATTGTATAAGGAACAAAACTGAGA
GAAAAAATCTAAAAGGCCGCCTATGATTGAATGGAAAGATTTTTTTTAGTTGAATTTAAATAATGTGACT
TGGGGGAGCCTTTACAAAGAGTCTTTATACCTCCCTTCAGCTTCCTCATTTTCCCTTGGATTACTTTTGC
TCAATTAAATATGAATTTCCT
CALM3
>gi|4502549|ref|NP_001734.1| calmodulin [Homo sapiens] (SEQ ID NO: 19)
MADQLTEEQIAEFKEAFSLFDKDGDGTITTKELGTVMRSLGQNPTEAELQDMINEVDADGNGTIDEPEFL
TMMARKMKDTDSEEEIREAFRVFDKDGNGYISAAELRHVMTNLGEKLTDEEVDEMIREADIDGDGQVNYE
EFVQMMTAK
>gi|58218967|ref|NM_005184.2| Homo sapiens calmodulin 3 (phosphorylase
kinase, delta) (CALM3), mRNA (SEQ ID NO: 20)
GGCGGGGCGCGCGCGGCGGCCGTTGAGGGACCGTTGGGGCGGGAGGCGGCGGCGGCGGCGGCGCGCGCTG
CGGGCAGTGAGTGTGGAGGCGCGGACGCGCGGCGGAGCTGGAACTGCTGCAGCTGCTGCCGCCGCCGGAG
GAACCTTGATCCCCGTGCTCCGGACACCCCGGGCCTCGCCATGGCTGACCAGCTGACTGAGGAGCAGATT
GCAGAGTTCAAGGAGGCCTTCTCCCTCTTTGACAAGGATGGAGATGGCACTATCACCACCAAGGAGTTGG
GGACAGTGATGAGATCCCTGGGACAGAACCCCACTGAAGCAGAGCTGCAGGATATGATCAATGAGGTGGA
TGCAGATGGGAACGGGACCATTGACTTCCCGGAGTTCCTGACCATGATGGCCAGAAAGATGAAGGACACA
GACAGTGAGGAGGAGATCCGAGAGGCGTTCCGTGTCTTTGACAAGGATGGGAATGGCTACATCAGCGCCG
CAGAGCTGCGTCACGTAATGACGAACCTGGGGGAGAAGCTGACCGATGAGGAGGTGGATGAGATGATCAG
GGAGGCTGACATCGATGGAGATGGCCAGGTCAATTATGAAGAGTTTGTACAGATGATGACTGCAAAGTGA
AGGCCCCCCGGGCAGCTGGCGATGCCCGTTCTCTTGATCTCTCTCTTCTCGCGCGCGCACTCTCTCTTCA
ACACTCCCCTGCGTACCCCGGTTCTAGCAAACACCAATTGATTGACTGAGAATCTGATAAAGCAACAAAA
GATTTGTCCCAAGCTGCATGATTGCTCTTTCTCCTTCTTCCCTGAGTCTCTCTCCATGCCCCTCATCTCT
TCCTTTTGCCCTCGCCTCTTCCATCCATGTCTTCCAAGGCCTGATGCATTCATAAGTTGAAGCCCTCCCC
AGATCCCCTTGGGGAGCCTCTGCCCTCCTCCAGCCCGGATGGCTCTCCTCCATTTTGGTTTGTTTCCTCT
TGTTTGTCATCTTATTTTGGGTGCTGGGGTGGCTGCCAGCCCTGTCCCGGGACCTGCTGGGAGGGACAAG
AGGCCCTCCCCCAGGCAGAAGAGCATGCCCTTTGCCGTTGCATGCAACCAGCCCTGTGATTCCACGTGCA
GATCCCAGCAGCCTGTTGGGGCAGGGGTGCCAAGAGAGGCATTCCAGAAGGACTGAGGGGGCGTTGAGGA
ATTGTGGCGTTGACTGGATGTGGCCCAGGAGGGGGTCGAGGGGGCCAACTCACAGAAGGGGACTGACAGT
GGGCAACACTCACATCCCACTGGCTGCTGTTCTGAAACCATCTGATTGGCTTTCTGAGGTTTGGCTGGGT
GGGGACTGCTCATTTGGCCACTCTGCAAATTGGACTTGCCCGCGTTCCTGAAGCGCTCTCGAGCTGTTCT
GTAAATACCTGGTGCTAACATCCCATGCCGCTCCCTCCTCACGATGCACCCACCGCCCTGAGGGCCCGTC
CTAGGAATGGATGTGGGGATGGTCGCTTTGTAATGTGCTGGTTCTCTTTTTTTTTCTTTCCCCTCTATGG
CCCTTAAGACTTTCATTTTGTTCAGAACCATGCTGGGCTAGCTAAAGGGTGGGGAGAGGGAAGATGGGCC
CCACCACGCTCTCAAGAGAACGCACCTGCAATAAAACAGTCTTGTCGGCCAGCTGCCCAGGGGACGGCAG
CTACAGCAGCCTCTGCGTCCTGGTCCGCCAGCACCTCCCGCTTCTCCGTGGTGACTTGGCGCCGCTTCCT
CACATCTGTGCTCCGTGCCCTCTTCCCTGCCTCTTCCCTCGCCCACCTGCCTGCCCCCATACTCCCCCAG
CGGAGAGCATGATCCGTGCCCTTGCTTCTGACTTTCGCCTCTGGGACAAGTAAGTCAATGTGGGCAGTTC
AGTCGTCTGGGTTTTTTCCCCTTTTCTGTTCATTTCATCTGGCTCCCCCCACCACCTCCCCACCCCACCC
CCCACCCCCTGCTTCCCCTCACTGCCCAGGTCGATCAAGTGGCTTTTCCTGGGACCTGCCCAGCTTTGAG
AATCTCTTCTCATCCACCCTCTGGCACCCAGCCTCTGAGGGAAGGAGGGATGGGGCATAGTGGGAGACCC
AGCCAAGAGCTGAGGGTAAGGGCAGGTAGGCGTGAGGCTGTGGACATTTTCGGAATGTTTTGGTTTTGTT
TTTTTTAAACCGGGCAATATTGTGTTCAGTTCAAGCTGTGAAGAAAAATATATATCAATGTTTTCCAATA
AAATACAGTGACTACCTGAAAAAAAAAAAAAAAAAAA
CD247
>gi|37595565|ref|NP_932170.1| T-cell surface glycoprotein CD3 zeta chain
isoform 1 precursor [Homo sapiens] (SEQ ID NO: 21)
MKWKALFTAAILQAQLPITEAQSFGLLDPKLCYLLDGILFIYGVILTALFLRVKFSRSADAPAYQQGQNQ
LYNELNLGRREEYDVLDKRRGRDPEMGGKPQRRKNPQEGLYNELQKDKMAEAYSEIGMKGERRRGKGHDG
LYQGLSTATKDTYDALHMQALPPR
>gi|4557431|ref|NP_000725.1| T-cell surface glycoprotein CD3 zeta chain
isoform 2 precursor [Homo sapiens] (SEQ ID NO: 22)
MKWKALFTAAILQAQLPITEAQSFGLLDPKLCYLLDGILFIYGVILTALFLRVKFSRSADAPAYQQGQNQ
LYNELNLGRREEYDVLDKRRGRDPEMGGKPRRKNPQEGLYNELQKDKMAEAYSEIGMKGERRRGKGHDGL
YQGLSTATKDTYDALHMQALPPR
>gi|166362721|ref|NM_198053.2| Homo sapiens CD247 molecule (CD247),
transcript variant 1, mRNA (SEQ ID NO: 23)
TGCTTTCTCAAAGGCCCCACAGTCCTCCACTTCCTGGGGAGGTAGCTGCAGAATAAAACCAGCAGAGACT
CCTTTTCTCCTAACCGTCCCGGCCACCGCTGCCTCAGCCTCTGCCTCCCAGCCTCTTTCTGAGGGAAAGG
ACAAGATGAAGTGGAAGGCGCTTTTCACCGCGGCCATCCTGCAGGCACAGTTGCCGATTACAGAGGCACA
GAGCTTTGGCCTGCTGGATCCCAAACTCTGCTACCTGCTGGATGGAATCCTCTTCATCTATGGTGTCATT
CTCACTGCCTTGTTCCTGAGAGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGGACAAGAGACGTGGCCG
GGACCCTGAGATGGGGGGAAAGCCGCAGAGAAGGAAGAACCCTCAGGAAGGCCTGTACAATGAACTGCAG
AAAGATAAGATGGCGGAGGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCACATGCAGGCCCTGCC
CCCTCGCTAACAGCCAGGGGATTTCACCACTCAAAGGCCAGACCTGCAGACGCCCAGATTATGAGACACA
GGATGAAGCATTTACAACCCGGTTCACTCTTCTCAGCCACTGAAGTATTCCCCTTTATGTACAGGATGCT
TTGGTTATATTTAGCTCCAAACCTTCACACACAGACTGTTGTCCCTGCACTCTTTAAGGGAGTGTACTCC
CAGGGCTTACGGCCCTGGCCTTGGGCCCTCTGGTTTGCCGGTGGTGCAGGTAGACCTGTCTCCTGGCGGT
TCCTCGTTCTCCCTGGGAGGCGGGCGCACTGCCTCTCACAGCTGAGTTGTTGAGTCTGTTTTGTAAAGTC
CCCAGAGAAAGCGCAGATGCTAGCACATGCCCTAATGTCTGTATCACTCTGTGTCTGAGTGGCTTCACTC
CTGCTGTAAATTTGGCTTCTGTTGTCACCTTCACCTCCTTTCAAGGTAACTGTACTGGGCCATGTTGTGC
CTCCCTGGTGAGAGGGCCGGGCAGAGGGGCAGATGGAAAGGAGCCTAGGCCAGGTGCAACCAGGGAGCTG
CAGGGGCATGGGAAGGTGGGCGGGCAGGGGAGGGTCAGCCAGGGCCTGCGAGGGCAGCGGGAGCCTCCCT
GCCTCAGGCCTCTGTGCCGCACCATTGAACTGTACCATGTGCTACAGGGGCCAGAAGATGAACAGACTGA
CCTTGATGAGCTGTGCACAAAGTGGCATAAAAAACATGTGGTTACACAGTGTGAATAAAGTGCTGCGGAG
CAAGAGGAGGCCGTTGATTCACTTCACGCTTTCAGCGAATGACAAAATCATCTTTGTGAAGGCCTCGCAG
GAAGACCCAACACATGGGACCTATAACTGCCCAGCGGACAGTGGCAGGACAGGAAAAACCCGTCAATGTA
CTAGGATACTGCTGCGTCATTACAGGGCACAGGCCATGGATGGAAAACGCTCTCTGCTCTGCTTTTTTTC
TACTGTTTTAATTTATACTGGCATGCTAAAGCCTTCCTATTTTGCATAATAAATGCTTCAGTGAAAATGC
AAAAAAAAAA
>gi|166362722|ref|NM_000734.3| Homo sapiens CD247 molecule (CD247),
transcript variant 2, mRNA (SEQ ID NO: 24)
TGCTTTCTCAAAGGCCCCACAGTCCTCCACTTCCTGGGGAGGTAGCTGCAGAATAAAACCAGCAGAGACT
CCTTTTCTCCTAACCGTCCCGGCCACCGCTGCCTCAGCCTCTGCCTCCCAGCCTCTTTCTGAGGGAAAGG
ACAAGATGAAGTGGAAGGCGCTTTTCACCGCGGCCATCCTGCAGGCACAGTTGCCGATTACAGAGGCACA
GAGCTTTGGCCTGCTGGATCCCAAACTCTGCTACCTGCTGGATGGAATCCTCTTCATCTATGGTGTCATT
CTCACTGCCTTGTTCCTGAGAGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCCTGTACAATGAACTGCAGAAA
GATAAGATGGCGGAGGCCTACAGTGAGATTGGGATGAAAGGCGAGCGCCGGAGGGGCAAGGGGCACGATG
GCCTTTACCAGGGTCTCAGTACAGCCACCAAGGACACCTACGACGCCCTTCACATGCAGGCCCTGCCCCC
TCGCTAACAGCCAGGGGATTTCACCACTCAAAGGCCAGACCTGCAGACGCCCAGATTATGAGACACAGGA
TGAAGCATTTACAACCCGGTTCACTCTTCTCAGCCACTGAAGTATTCCCCTTTATGTACAGGATGCTTTG
GTTATATTTAGCTCCAAACCTTCACACACAGACTGTTGTCCCTGCACTCTTTAAGGGAGTGTACTCCCAG
GGCTTACGGCCCTGGCCTTGGGCCCTCTGGTTTGCCGGTGGTGCAGGTAGACCTGTCTCCTGGCGGTTCC
TCGTTCTCCCTGGGAGGCGGGCGCACTGCCTCTCACAGCTGAGTTGTTGAGTCTGTTTTGTAAAGTCCCC
AGAGAAAGCGCAGATGCTAGCACATGCCCTAATGTCTGTATCACTCTGTGTCTGAGTGGCTTCACTCCTG
CTGTAAATTTGGCTTCTGTTGTCACCTTCACCTCCTTTCAAGGTAACTGTACTGGGCCATGTTGTGCCTC
CCTGGTGAGAGGGCCGGGCAGAGGGGCAGATGGAAAGGAGCCTAGGCCAGGTGCAACCAGGGAGCTGCAG
GGGCATGGGAAGGTGGGCGGGCAGGGGAGGGTCAGCCAGGGCCTGCGAGGGCAGCGGGAGCCTCCCTGCC
TCAGGCCTCTGTGCCGCACCATTGAACTGTACCATGTGCTACAGGGGCCAGAAGATGAACAGACTGACCT
TGATGAGCTGTGCACAAAGTGGCATAAAAAACATGTGGTTACACAGTGTGAATAAAGTGCTGCGGAGCAA
GAGGAGGCCGTTGATTCACTTCACGCTTTCAGCGAATGACAAAATCATCTTTGTGAAGGCCTCGCAGGAA
GACCCAACACATGGGACCTATAACTGCCCAGCGGACAGTGGCAGGACAGGAAAAACCCGTCAATGTACTA
GGATACTGCTGCGTCATTACAGGGCACAGGCCATGGATGGAAAACGCTCTCTGCTCTGCTTTTTTTCTAC
TGTTTTAATTTATACTGGCATGCTAAAGCCTTCCTATTTTGCATAATAAATGCTTCAGTGAAAATGCAAA
AAAAAAA
HDAC1
>gi|13128860|ref|NP_004955.2| histone deacetylase1 [Homo sapiens] (SEQ
ID NO: 25)
MAQTQGTRRKVCYYYDGDVGNYYYGQGHPMKPHRIRMTHNLLLNYGLYRKMEIYRPHKANAEEMTKYHSD
DYIKFLRSIRPDNMSEYSKQMQRFNVGEDCPVFDGLFEFCQLSTGGSVASAVKLNKQQTDIAVNWAGGLH
HAKKSEASGFCYVNDIVLAILELLKYHQRVLYIDIDIHHGDGVEEAFYTTDRVMTVSFHKYGEYFPGTGD
LRDIGAGKGKYYAVNYPLRDGIDDESYEAIFKPVMSKVMEMFQPSAVVLQCGSDSLSGDRLGCFNLTIKG
HAKCVEFVKSFNLPMLMLGGGGYTIRNVARCWTYETAVALDTEIPNELPYNDYFEYFGPDFKLHISPSNM
TNQNTNEYLEKIKQRLFENLRMLPHAPGVQMQAIPEDAIPEESGDEDEDDPDKRISICSSDKRIACEEEF
SDSEEEGEGGRKNSSNFKKAKRVKTEDEKEKDPEEKKEVTEEEKTKEEKPEAKGVKEEVKLA
>gi|13128859|ref|NM_004964.2| Homo sapiens histone deacetylase 1
(HDAC1), mRNA (SEQ ID NO: 26)
GAGCGGAGCCGCGGGCGGGAGGGCGGACGGACCGACTGACGGTAGGGACGGGAGGCGAGCAAGATGGCGC
AGACGCAGGGCACCCGGAGGAAAGTCTGTTACTACTACGACGGGGATGTTGGAAATTACTATTATGGACA
AGGCCACCCAATGAAGCCTCACCGAATCCGCATGACTCATAATTTGCTGCTCAACTATGGTCTCTACCGA
AAAATGGAAATCTATCGCCCTCACAAAGCCAATGCTGAGGAGATGACCAAGTACCACAGCGATGACTACA
TTAAATTCTTGCGCTCCATCCGTCCAGATAACATGTCGGAGTACAGCAAGCAGATGCAGAGATTCAACGT
TGGTGAGGACTGTCCAGTATTCGATGGCCTGTTTGAGTTCTGTCAGTTGTCTACTGGTGGTTCTGTGGCA
AGTGCTGTGAAACTTAATAAGCAGCAGACGGACATCGCTGTGAATTGGGCTGGGGGCCTGCACCATGCAA
AGAAGTCCGAGGCATCTGGCTTCTGTTACGTCAATGATATCGTCTTGGCCATCCTGGAACTGCTAAAGTA
TCACCAGAGGGTGCTGTACATTGACATTGATATTCACCATGGTGACGGCGTGGAAGAGGCCTTCTACACC
ACGGACCGGGTCATGACTGTGTCCTTTCATAAGTATGGAGAGTACTTCCCAGGAACTGGGGACCTACGGG
ATATCGGGGCTGGCAAAGGCAAGTATTATGCTGTTAACTACCCGCTCCGAGACGGGATTGATGACGAGTC
CTATGAGGCCATTTTCAAGCCGGTCATGTCCAAAGTAATGGAGATGTTCCAGCCTAGTGCGGTGGTCTTA
CAGTGTGGCTCAGACTCCCTATCTGGGGATCGGTTAGGTTGCTTCAATCTAACTATCAAAGGACACGCCA
AGTGTGTGGAATTTGTCAAGAGCTTTAACCTGCCTATGCTGATGCTGGGAGGCGGTGGTTACACCATTCG
TAACGTTGCCCGGTGCTGGACATATGAGACAGCTGTGGCCCTGGATACGGAGATCCCTAATGAGCTTCCA
TACAATGACTACTTTGAATACTTTGGACCAGATTTCAAGCTCCACATCAGTCCTTCCAATATGACTAACC
AGAACACGAATGAGTACCTGGAGAAGATCAAACAGCGACTGTTTGAGAACCTTAGAATGCTGCCGCACGC
ACCTGGGGTCCAAATGCAGGCGATTCCTGAGGACGCCATCCCTGAGGAGAGTGGCGATGAGGACGAAGAC
GACCCTGACAAGCGCATCTCGATCTGCTCCTCTGACAAACGAATTGCCTGTGAGGAAGAGTTCTCCGATT
CTGAAGAGGAGGGAGAGGGGGGCCGCAAGAACTCTTCCAACTTCAAAAAAGCCAAGAGAGTCAAAACAGA
GGATGAAAAAGAGAAAGACCCAGAGGAGAAGAAAGAAGTCACCGAAGAGGAGAAAACCAAGGAGGAGAAG
CCAGAAGCCAAAGGGGTCAAGGAGGAGGTCAAGTTGGCCTGAATGGACCTCTCCAGCTCTGGCTTCCTGC
TGAGTCCCTCACGTTTCTTCCCCAACCCCTCAGATTTTATATTTTCTATTTCTCTGTGTATTTATATAAA
AATTTATTAAATATAAATATCCCCAGGGACAGAAACCAAGGCCCCGAGCTCAGGGCAGCTGTGCTGGGTG
AGCTCTTCCAGGAGCCACCTTGCCACCCATTCTTCCCGTTCTTAACTTTGAACCATAAAGGGTGCCAGGT
CTGGGTGAAAGGGATACTTTTATGCAACCATAAGACAAACTCCTGAAATGCCAAGTGCCTGCTTAGTAGC
TTTGGAAAGGTGCCCTTATTGAACATTCTAGAAGGGGTGGCTGGGTCTTCAAGGATCTCCTGTTTTTTTC
AGGCTCCTAAAGTAACATCAGCCATTTTTAGATTGGTTCTGTTTTCGTACCTTCCCACTGGCCTCAAGTG
AGCCAAGAAACACTGCCTGCCCTCTGTCTGTCTTCTCCTAATTCTGCAGGTGGAGGTTGCTAGTCTAGTT
TCCTTTTTGAGATACTATTTTCATTTTTGTGAGCCTCTTTGTAATAAAATGGTACATTTCT
IFNA5
>gi|4504597|ref|NP_002160.1| interferon alpha-5 precursor [Homo
sapiens] (SEQ ID NO: 27)
MALPFVLLMALVVLNCKSICSLGCDLPQTHSLSNRRTLMIMAQMGRISPFSCLKDRHDFGFPQEEFDGNQ
FQKAQAISVLHEMIQQTFNLFSTKDSSATWDETLLDKFYTELYQQLNDLEACMMQEVGVEDTPLMNVDSI
LTVRKYFQRITLYLTEKKYSPCAWEVVRAEIMRSFSLSANLQERLRRKE
>gi|291463310|ref|NM_002169.2| Homo sapiens interferon, alpha 5 (IFNA5),
mRNA (SEQ ID NO: 28)
GCCCAAGGTTCAGGGTCACTCAATCTCAACAGCCCAGAAGCATCTGCAACCTCCCCAATGGCCTTGCCCT
TTGTTTTACTGATGGCCCTGGTGGTGCTCAACTGCAAGTCAATCTGTTCTCTGGGCTGTGATCTGCCTCA
GACCCACAGCCTGAGTAACAGGAGGACTTTGATGATAATGGCACAAATGGGAAGAATCTCTCCTTTCTCC
TGCCTGAAGGACAGACATGACTTTGGATTTCCTCAGGAGGAGTTTGATGGCAACCAGTTCCAGAAGGCTC
AAGCCATCTCTGTCCTCCATGAGATGATCCAGCAGACCTTCAATCTCTTCAGCACAAAGGACTCATCTGC
TACTTGGGATGAGACACTTCTAGACAAATTCTACACTGAACTTTACCAGCAGCTGAATGACCTGGAAGCC
TGTATGATGCAGGAGGTTGGAGTGGAAGACACTCCTCTGATGAATGTGGACTCTATCCTGACTGTGAGAA
AATACTTTCAAAGAATCACCCTCTATCTGACAGAGAAGAAATACAGCCCTTGTGCATGGGAGGTTGTCAG
AGCAGAAATCATGAGATCCTTCTCTTTATCAGCAAACTTGCAAGAAAGATTAAGGAGGAAGGAATGAAAA
CTGGTTCAACATCGAAATGATTCTCATTGACTAGTACACCATTTCACACTTCTTGAGTTCTGCCGTTTCA
FOS
>gi|4885241|ref|NP_005243.1| proto-oncogene c-Fos [Homo sapiens] (SEQ ID
NO: 29)
MMFSGFNADYEASSSRCSSASPAGDSLSYYHSPADSFSSMGSPVNAQDFCTDLAVSSANFIPTVTAISTS
PDLQWLVQPALVSSVAPSQTRAPHPFGVPAPSAGAYSRAGVVKTMTGGRAQSIGRRGKVEQLSPEEEEKR
RIRRERNKMAAAKCRNRRRELTDTLQAETDQLEDEKSALQTEIANLLKEKEKLEFILAAHRPACKIPDDL
GFPEEMSVASLDLTGGLPEVATPESEEAFTLPLLNDPEPKPSVEPVKSISSMELKTEPFDDFLFPASSRP
SGSETARSVPDMDLSGSFYAADWEPLHSGSLGMGPMATELEPLCTPVVTCTPSCTAYTSSFVFTYPEADS
FPSCAAAHRKGSSSNEPSSDSLSSPTLLAL
>gi|254750707|ref|NM_005252.3| Homo sapiens FBJ murine osteosarcoma
viral oncogene homolog (FOS), mRNA (SEQ ID NO: 30)
ATTCATAAAACGCTTGTTATAAAAGCAGTGGCTGCGGCGCCTCGTACTCCAACCGCATCTGCAGCGAGCA
TCTGAGAAGCCAAGACTGAGCCGGCGGCCGCGGCGCAGCGAACGAGCAGTGACCGTGCTCCTACCCAGCT
CTGCTCCACAGCGCCCACCTGTCTCCGCCCCTCGGCCCCTCGCCCGGCTTTGCCTAACCGCCACGATGAT
GTTCTCGGGCTTCAACGCAGACTACGAGGCGTCATCCTCCCGCTGCAGCAGCGCGTCCCCGGCCGGGGAT
AGCCTCTCTTACTACCACTCACCCGCAGACTCCTTCTCCAGCATGGGCTCGCCTGTCAACGCGCAGGACT
TCTGCACGGACCTGGCCGTCTCCAGTGCCAACTTCATTCCCACGGTCACTGCCATCTCGACCAGTCCGGA
CCTGCAGTGGCTGGTGCAGCCCGCCCTCGTCTCCTCCGTGGCCCCATCGCAGACCAGAGCCCCTCACCCT
TTCGGAGTCCCCGCCCCCTCCGCTGGGGCTTACTCCAGGGCTGGCGTTGTGAAGACCATGACAGGAGGCC
GAGCGCAGAGCATTGGCAGGAGGGGCAAGGTGGAACAGTTATCTCCAGAAGAAGAAGAGAAAAGGAGAAT
CCGAAGGGAAAGGAATAAGATGGCTGCAGCCAAATGCCGCAACCGGAGGAGGGAGCTGACTGATACACTC
CAAGCGGAGACAGACCAACTAGAAGATGAGAAGTCTGCTTTGCAGACCGAGATTGCCAACCTGCTGAAGG
AGAAGGAAAAACTAGAGTTCATCCTGGCAGCTCACCGACCTGCCTGCAAGATCCCTGATGACCTGGGCTT
CCCAGAAGAGATGTCTGTGGCTTCCCTTGATCTGACTGGGGGCCTGCCAGAGGTTGCCACCCCGGAGTCT
GAGGAGGCCTTCACCCTGCCTCTCCTCAATGACCCTGAGCCCAAGCCCTCAGTGGAACCTGTCAAGAGCA
TCAGCAGCATGGAGCTGAAGACCGAGCCCTTTGATGACTTCCTGTTCCCAGCATCATCCAGGCCCAGTGG
CTCTGAGACAGCCCGCTCCGTGCCAGACATGGACCTATCTGGGTCCTTCTATGCAGCAGACTGGGAGCCT
CTGCACAGTGGCTCCCTGGGGATGGGGCCCATGGCCACAGAGCTGGAGCCCCTGTGCACTCCGGTGGTCA
CCTGTACTCCCAGCTGCACTGCTTACACGTCTTCCTTCGTCTTCACCTACCCCGAGGCTGACTCCTTCCC
CAGCTGTGCAGCTGCCCACCGCAAGGGCAGCAGCAGCAATGAGCCTTCCTCTGACTCGCTCAGCTCACCC
ACGCTGCTGGCCCTGTGAGGGGGCAGGGAAGGGGAGGCAGCCGGCACCCACAAGTGCCACTGCCCGAGCT
GGTGCATTACAGAGAGGAGAAACACATCTTCCCTAGAGGGTTCCTGTAGACCTAGGGAGGACCTTATCTG
TGCGTGAAACACACCAGGCTGTGGGCCTCAAGGACTTGAAAGCATCCATGTGTGGACTCAAGTCCTTACC
TCTTCCGGAGATGTAGCAAAACGCATGGAGTGTGTATTGTTCCCAGTGACACTTCAGAGAGCTGGTAGTT
AGTAGCATGTTGAGCCAGGCCTGGGTCTGTGTCTCTTTTCTCTTTCTCCTTAGTCTTCTCATAGCATTAA
CTAATCTATTGGGTTCATTATTGGAATTAACCTGGTGCTGGATATTTTCAAATTGTATCTAGTGCAGCTG
ATTTTAACAATAACTACTGTGTTCCTGGCAATAGTGTGTTCTGATTAGAAATGACCAATATTATACTAAG
AAAAGATACGACTTTATTTTCTGGTAGATAGAAATAAATAGCTATATCCATGTACTGTAGTTTTTCTTCA
ACATCAATGTTCATTGTAATGTTACTGATCATGCATTGTTGAGGTGGTCTGAATGTTCTGACATTAACAG
TTTTCCATGAAAACGTTTTATTGTGTTTTTAATTTATTTATTAAGATGGATTCTCAGATATTTATATTTT
TATTTTATTTTTTTCTACCTTGAGGTCTTTTGACATGTGGAAAGTGAATTTGAATGAAAAATTTAAGCAT
TGTTTGCTTATTGTTCCAAGACATTGTCAATAAAAGCATTTAAGTTGAATGCGACCAA

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

What is claimed is:

1. A method for diagnosing lupus, determining the likelihood of developing lupus, or determining the severity of lupus in a subject, said method comprising determining an expression level of one or more genes in a biological sample from said subject, wherein an increased or decreased level for said one or more genes in said biological sample, as compared to a control, is indicative of the presence of lupus, an increased likelihood of developing lupus, or an increased severity of lupus; and wherein said genes are selected from the group consisting of: interferon alpha 1 (IFNA1); CD247 molecule (CD3ζ) (CD247); cAMP responsive element modulator (CREM); histone deacetylase 1 (HDAC1); nuclear factor of activated T cells, cytoplasmic, calcineurin-dependent 2 (NFATC2); prostaglandin-endoperoxide synthase 2 (prostaglandin G/H synthase and cyclooxygenase) (PTGS2); interferon alpha 5 (IFNA5); cytotoxic T-lymphocyte-associated protein 4 (CTLA4); intercellular adhesion molecule 1 (CD54), human rhinovirus receptor (ICAM1); programmed cell death 1 (PDCD1); rho-associated, coiled-coil containing protein kinase 1 (ROCK1); interleukin 10 (IL10); CD40 ligand (TNF superfamily, member 5, hyper-IgM syndrome) (CD40LG); Fas ligand (TNF superfamily member 6) (FASLG); interferon gamma (IFNG); protein phosphatase 2 (formerly 2A), catalytic subunit, alpha isoform (PPP2CA); spleen tyrosine kinase (SYK); interleukin 23, alpha subunit p19 (IL23A); CD44 molecule (Indian blood group) (CD44); Fc fragment of IgE, high affinity 1, receptor for gamma polypeptide (FCER1G); interleukin 17A (IL17A); protein phosphatase 2 (formerly 2A), catalytic subunit, beta isoform (PPP2CB); ezrin (EZR); v3 variant of CD44 (CD44V3); V-fos FBJ murine osteosarcoma viral oncogene homolog (FOS); interleukin 17F (IL17F); protein kinase, cAMP-dependent, regulatory, type I, beta (PRKAR1B); v6 variant of CD44 (CD44V6); Forkhead box P3 (FOXP3); interleukin 2 (IL2); protein kinase, cAMP-dependent, regulatory, type II, beta (PRKAR2B); CD70 molecule (CD70); GATA binding protein 3 (GATA3); interleukin 21 (IL21); Protein kinase C, delta (PRKCD); calmodulin 3 (phosphorylase kinase, delta) (CALM3); cAMP response element binding protein 1 (CREB1); V-rel reticuloendotheliosis viral oncogene homolog A, nuclear factor of kappa light polypeptide gene enhancer in B-cells, p65 (avian) (RELA); interleukin 6 (IL6); and protein kinase C, theta (PRKCQ).

2. The method of claim 1, further comprising contacting said biological sample with one or more binding agents capable of specifically binding said one or more genes or a protein encoded by said one or more genes.

3. The method of claim 1, further comprising, prior to determining said expression level, extracting mRNA from said sample and reverse transcribing said mRNA into cDNA to obtain a treated biological sample.

4. The method of claim 3, further comprising contacting said treated biological sample with one or more binding agents capable of specifically binding said one or more genes or a protein encoded by said one or more genes.

5. The method of claim 1, wherein said expression level is mRNA expression level, cDNA expression level, or protein expression level.

6. The method of claim 1, wherein said biological sample comprises mRNA, cDNA, and/or protein from said subject.

7. The method of claim 1, wherein said one or more genes comprise IL10.

8. The method of claim 1, wherein said one or more genes are selected from the group consisting of IL10, IFNA5, CD44, CALM3, CD44V3, FOS, CD247, and HDAC1.

9. The method of claim 1, wherein said expression level is determined by one or more of a hybridization assay, an amplification-based assay, or fluorescence in situ hybridization.

10. The method of claim 1, wherein said lupus is systemic lupus erythematosus, complement deficiency syndrome, cutaneous lupus erythematosus, drug-induced lupus erythematosus, or neonatal lupus.

11. The method of claim 10, wherein said lupus is cutaneous lupus erythematosus selected from the group consisting of chronic cutaneous lupus erythematosus, discoid lupus erythematosus, chilblain lupus erythematosus, lupus erythematosus-lichen planus overlap syndrome, lupus erythematosus panniculitis, subacute cutaneous lupus erythematosus, tumid lupus erythematosus, and verrucous lupus erythematosus.

12. A method for treating lupus in a subject, said method comprising:
(a) administering to said subject a therapeutically effective amount of a therapeutic agent; and
(b) determining an expression level of one or more genes in a biological sample from said subject, wherein an increased or decreased level for said one or more genes in said biological sample, as compared to a control, is indicative of an increased severity of lupus, thereby indicating administration of an increased dosage of said therapeutic agent or administration of a different therapeutic agent to treat said subject; and wherein said genes are selected from the group consisting of: interferon alpha 1 (IFNA1); CD247 molecule (CD3ζ) (CD247); cAMP responsive element modulator (CREM); histone deacetylase 1 (HDAC1); nuclear factor of activated T cells, cytoplasmic, calcineurin-dependent 2 (NFATC2); prostaglandin-endoperoxide synthase 2 (prostaglandin G/H synthase and cyclooxygenase) (PTGS2); interferon alpha 5 (IFNA5); cytotoxic T-lymphocyte-associated protein 4 (CTLA4); intercellular adhesion molecule 1 (CD54), human rhinovirus receptor (ICAM1); programmed cell death 1 (PDCD1); rho-associated, coiled-coil containing protein kinase 1 (ROCK1); interleukin 10 (IL10); CD40 ligand (TNF superfamily, member 5, hyper-IgM syndrome) (CD40LG); Fas ligand (TNF superfamily member 6) (FASLG); interferon gamma (IFNG); protein phosphatase 2 (formerly 2A), catalytic subunit, alpha isoform (PPP2CA); spleen tyrosine kinase (SYK); interleukin 23, alpha subunit p19 (IL23A); CD44 molecule (Indian blood group) (CD44); Fc fragment of IgE, high affinity 1, receptor for gamma polypeptide (FCER1G); interleukin 17A (IL17A); protein phosphatase 2 (formerly 2A), catalytic subunit, beta isoform (PPP2CB); ezrin (EZR); v3 variant of CD44 (CD44V3); V-fos FBJ murine osteosarcoma viral oncogene homolog (FOS); interleukin 17F (IL17F); protein kinase, cAMP-dependent, regulatory, type I, beta (PRKAR1B); v6 variant of CD44 (CD44V6); Forkhead box P3 (FOXP3); interleukin 2 (IL2); protein kinase, cAMP-dependent, regulatory, type II, beta (PRKAR2B); CD70 molecule (CD70); GATA binding protein 3 (GATA3); interleukin 21 (IL21); Protein kinase C, delta (PRKCD); calmodulin 3 (phosphorylase kinase, delta) (CALM3); cAMP response element binding protein 1 (CREB1); V-rel reticuloendotheliosis viral oncogene homolog A, nuclear factor of kappa light polypeptide gene enhancer in B-cells, p65 (avian) (RELA); interleukin 6 (IL6); and protein kinase C, theta (PRKCQ).

13. The method of claim 12, wherein said therapeutic agent is acetaminophen, a nonsteroidal anti-inflammatory drug, a corticosteroid, an antimalarial, or an immunosuppressant.

14. The method of claim 12, wherein said lupus is systemic lupus erythematosus, complement deficiency syndrome, cutaneous lupus erythematosus, drug-induced lupus erythematosus, or neonatal lupus.

15. A method for diagnosing lupus, determining the likelihood of developing lupus, or determining the severity of lupus in a subject, said method comprising:
(a) contacting a biological sample from said subject with one or more binding agents capable of specifically binding one or more genes or a protein encoded by said one or more genes; and
(b) determining an expression level of said one or more genes in said biological sample, wherein an increased or decreased level for said one or more genes in said biological sample, as compared to a control, is indicative of the presence of lupus, an increased likelihood of developing lupus, or increased severity of lupus; and wherein said genes are selected from the group consisting of: interferon alpha 1 (IFNA1); CD247 molecule (CD3ζ) (CD247); cAMP responsive element modulator (CREM); histone deacetylase 1 (HDAC1); nuclear factor of activated T cells, cytoplasmic, calcineurin-dependent 2 (NFATC2); prostaglandin-endoperoxide synthase 2 (prostaglandin G/H synthase and cyclooxygenase) (PTGS2); interferon alpha 5 (IFNA5); cytotoxic T-lymphocyte-associated protein 4 (CTLA4); intercellular adhesion molecule 1 (CD54), human rhinovirus receptor (ICAM1); programmed cell death 1 (PDCD1); rho-associated, coiled-coil containing protein kinase 1 (ROCK1); interleukin 10 (IL10); CD40 ligand (TNF superfamily, member 5, hyper-IgM syndrome) (CD40LG); Fas ligand (TNF superfamily member 6) (FASLG); interferon gamma (IFNG); protein phosphatase 2 (formerly 2A), catalytic subunit, alpha isoform (PPP2CA); spleen tyrosine kinase (SYK); interleukin 23, alpha subunit p19 (IL23A); CD44 molecule (Indian blood group) (CD44); Fc fragment of IgE, high affinity 1, receptor for gamma polypeptide (FCER1G); interleukin 17A (IL17A); protein phosphatase 2 (formerly 2A), catalytic subunit, beta isoform (PPP2CB); ezrin (EZR); v3 variant of CD44 (CD44V3); V-fos FBJ murine osteosarcoma viral oncogene homolog (FOS); interleukin 17F (IL17F); protein kinase, cAMP-dependent, regulatory, type I, beta (PRKAR1B); v6 variant of CD44 (CD44V6); Forkhead box P3 (FOXP3); interleukin 2 (IL2); protein kinase, cAMP-dependent, regulatory, type II, beta (PRKAR2B); CD70 molecule (CD70); GATA binding protein 3 (GATA3); interleukin 21 (IL21); Protein kinase C, delta (PRKCD); calmodulin 3 (phosphorylase kinase, delta) (CALM3); cAMP response element binding protein 1 (CREB1); V-rel reticuloendotheliosis viral oncogene homolog A, nuclear factor of kappa light polypeptide gene enhancer in B-cells, p65 (avian) (RELA); interleukin 6 (IL6); and protein kinase C, theta (PRKCQ).

16. The method of claim 15, further comprising, prior to contacting said sample, extracting mRNA from said sample and reverse transcribing said mRNA into cDNA.

17. The method of claim 15, wherein said expression level is mRNA expression level, cDNA expression level, or protein expression level.

18. A kit for diagnosing a subject having, or having a predisposition to develop, lupus, said kit comprising:
(a) one or more binding agents capable of specifically binding one or more genes or a protein encoded by said one or more genes; and
(b) instructions for use of said kit, wherein said genes are selected from the group consisting of: interferon alpha 1 (IFNA1); CD247 molecule (CD3ζ) (CD247); cAMP responsive element modulator (CREM); histone deacetylase 1 (HDAC1); nuclear factor of activated T cells, cytoplasmic, calcineurin-dependent 2 (NFATC2); prostaglandin-endoperoxide synthase 2 (prostaglandin G/H synthase and cyclooxygenase) (PTGS2); interferon alpha 5 (IFNA5); cytotoxic T-lymphocyte-associated protein 4 (CTLA4); intercellular adhesion molecule 1 (CD54), human rhinovirus receptor (ICAM1); programmed cell death 1 (PDCD1); rho-associated, coiled-coil containing protein kinase 1 (ROCK1); interleukin 10 (IL10); CD40 ligand (TNF superfamily, member 5, hyper-IgM syndrome) (CD40LG); Fas ligand (TNF superfamily member 6) (FASLG); interferon gamma (IFNG); protein phosphatase 2 (formerly 2A), catalytic subunit, alpha isoform (PPP2CA); spleen tyrosine kinase (SYK); interleukin 23, alpha subunit p19 (IL23A); CD44 molecule (Indian blood group) (CD44); Fc fragment of IgE, high affinity 1, receptor for gamma polypeptide (FCER1G); interleukin 17A (IL17A); protein phosphatase 2 (formerly 2A), catalytic subunit, beta isoform (PPP2CB); ezrin (EZR); v3 variant of CD44 (CD44V3); V-fos FBJ murine osteosarcoma viral oncogene homolog (FOS); interleukin 17F (IL17F); protein kinase, cAMP-dependent, regulatory, type I, beta (PRKAR1B); v6 variant of CD44 (CD44V6); Forkhead box P3 (FOXP3); interleukin 2 (IL2); protein kinase, cAMP-dependent, regulatory, type II, beta (PRKAR2B); CD70 molecule (CD70); GATA binding protein 3 (GATA3); interleukin 21 (IL21); Protein kinase C, delta (PRKCD); calmodulin 3 (phosphorylase kinase, delta) (CALM3); cAMP response element binding protein 1 (CREB1); V-rel reticuloendotheliosis viral oncogene homolog A, nuclear factor of kappa light polypeptide gene enhancer in B-cells, p65 (avian) (RELA); interleukin 6 (IL6); and protein kinase C, theta (PRKCQ).

19. The kit of claim 18, wherein said one or more binding agents are polynucleotides or polypeptides.

20. The kit of claim 19, wherein said one or more binding agents are polynucleotides, and each of said polynucleotides comprises a sequence that is substantially identical to the sequence of any one of SEQ ID NOs: 2, 11-18, 20, 23, 24, 26, 28, or 30, or a fragment thereof.

21. The kit of claim 19, wherein said one or more binding agents are polynucleotides, and each of said polynucleotides comprises a sequence that is substantially identical to a sequence that is substantially complementary to the sequence of any one of SEQ ID NOs: 2, 11-18, 20, 23, 24, 26, 28, or 30, or a fragment thereof.

22. The kit of claim 19, wherein said one or more binding agents are provided on a solid support.

23. The kit of claim 18, wherein said instructions comprise one or more metrics for a principal component analysis that indicates the diagnosis for lupus or the predisposition to develop lupus.
